SEQUENCE LISTING (157 sequences)

SEQ ID NO: 1 - protein, 619 aa, synthetic construct
  REGION 1..619: note = Description of sequence: Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 2 - protein, 509 aa, synthetic construct
  REGION 1..509: note = Description of sequence: Synthetic polypeptide
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 3 - protein, 244 aa, synthetic construct
  REGION 1..244: note = Description of sequence: Synthetic polypeptide
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRET

SEQ ID NO: 4 - protein, 537 aa, synthetic construct
  REGION 1..537: note = Description of sequence: Synthetic polypeptide
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYQPGENL

SEQ ID NO: 5 - protein, 270 aa, synthetic construct
  REGION 1..270: note = Description of sequence: Synthetic polypeptide
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRES

SEQ ID NO: 6 - protein, 635 aa, synthetic construct
  REGION 1..635: note = Description of sequence: Synthetic polypeptide
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN

SEQ ID NO: 7 - protein, 370 aa, synthetic construct
  REGION 1..370: note = Description of sequence: Synthetic polypeptide
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLL

SEQ ID NO: 8 - protein, 262 aa, synthetic construct
  REGION 1..262: note = Description of sequence: Synthetic polypeptide
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 9 - protein, 240 aa, synthetic construct
  REGION 1..240: note = Description of sequence: Synthetic polypeptide
MKVKKGGGGAGTATESAPGPSGQSVAPIPQPPAESESGSESEPDAGPGPRPGPLQRKQPIGPEDVLGLQRITGDYLCSPEENIYKIDFVRFKIRDMDSGTVLFEIKKPPVSERLPINRRDLDPNAGRFVRYQFTPAFLRLRQVGATVEFTVGDKPVNNFRMIERHYFRNQLLKSFDFHFGFCIPSSKNTCEHIYDFPPLSEELISEMIRHPYETQSDSFYFVDDRLVMHNKADYSYSGTP

SEQ ID NO: 10 - protein, 21 aa, synthetic construct
  REGION 1..21: note = Description of sequence: Synthetic peptide
MALPVTALLLPLALLLHAARP

SEQ ID NO: 11 - protein, 45 aa, synthetic construct
  REGION 1..45: note = Description of sequence: Synthetic polypeptide
TTTPAPRPPTPAPTIASQPLSLRPEACRPAAGGAVHTRGLDFACD

SEQ ID NO: 12 - protein, 21 aa, synthetic construct
  REGION 1..21: note = Description of sequence: Synthetic peptide
IYIWAPLAGTCGVLLLSLVIT

SEQ ID NO: 13 - skipped

SEQ ID NO: 14 - protein, 42 aa, synthetic construct
  REGION 1..42: note = Description of sequence: Synthetic polypeptide
KRGRKKLLYIFKQPFMRPVQTTQEEDGCSCRFPEEEEGGCEL

SEQ ID NO: 15 - protein, 112 aa, synthetic construct
  REGION 1..112: note = Description of sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 225 aa, synthetic construct
  REGION 1..225: note = Description of sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 45 aa, synthetic construct
  REGION 1..45: note = Description of sequence: Synthetic polypeptide
GHETGRLSGAADTQALLRNDQVYQPLRDRDDAQYSHLGGNWARNK

SEQ ID NO: 18 - protein, 55 aa, synthetic construct
  REGION 1..55: note = Description of sequence: Synthetic polypeptide
KNRKAKAKPVTRGAGAGGRQRGQNKERPPPVPNPDYEPIRKGQRDLYSGLNQRRI

SEQ ID NO: 19 - protein, 45 aa, synthetic construct
  REGION 1..45: note = Description of sequence: Synthetic polypeptide
GQDGVRQSRASDKQTLLPNDQLYQPLKDREDDQYSHLQGNQLRRN

SEQ ID NO: 20 - protein, 29 aa, synthetic construct
  REGION 1..29: note = Description of sequence: Synthetic peptide
APAYQQGQNQLYNELNLGRREEYDVLDKR

SEQ ID NO: 21 - protein, 28 aa, synthetic construct
  REGION 1..28: note = Description of sequence: Synthetic peptide
PRRKNPQEGLYNELQKDKMAEAYSEIGM

SEQ ID NO: 22 - protein, 29 aa, synthetic construct
  REGION 1..29: note = Description of sequence: Synthetic peptide
ERRRGKGHDGLYQGLSTATKDTYDALHMQ

SEQ ID NO: 23 - protein, 29 aa, synthetic construct
  REGION 1..29: note = Description of sequence: Synthetic peptide
DTQALLRNDQVYQPLRDRDDAQYSHLGGN

SEQ ID NO: 24 - protein, 28 aa, synthetic construct
  REGION 1..28: note = Description of sequence: Synthetic peptide
ERPPPVPNPDYEPIRKGQRDLYSGLNQR

SEQ ID NO: 25 - protein, 29 aa, synthetic construct
  REGION 1..29: note = Description of sequence: Synthetic peptide
DKQTLLPNDQLYQPLKDREDDQYSHLQGN

SEQ ID NO: 26 - protein, 112 aa, synthetic construct
  REGION 1..112: note = Description of sequence: Synthetic polypeptide
RVKFSRSADDTQALLRNDQVYQPLRDRDDAQYSHLGGNRGRDPEMGGKPRRKNPQEGLYNELQKDKMAEAYSEIGMKGERRRGKGHDGLYQGLSTATKDTYDALHMQALPPR

SEQ ID NO: 27 - protein, 111 aa, synthetic construct
  REGION 1..111: note = Description of sequence: Synthetic polypeptide
RVKFSRSADERPPPVPNPDYEPIRKGQRDLYSGLNQRRGRDPEMGGKPRRKNPQEGLYNELQKDKMAEAYSEIGMKGERRRGKGHDGLYQGLSTATKDTYDALHMQALPPR

SEQ ID NO: 28 - protein, 112 aa, synthetic construct
  REGION 1..112: note = Description of sequence: Synthetic polypeptide
RVKFSRSADDKQTLLPNDQLYQPLKDREDDQYSHLQGNRGRDPEMGGKPRRKNPQEGLYNELQKDKMAEAYSEIGMKGERRRGKGHDGLYQGLSTATKDTYDALHMQALPPR

SEQ ID NO: 29 - protein, 112 aa, synthetic construct
  REGION 1..112: note = Description of sequence: Synthetic polypeptide
RVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 30 - protein, 225 aa, synthetic construct
  REGION 1..225: note = Description of sequence: Synthetic polypeptide
RVKFSRSADAPAYQQGQNQLYNELNLGRREEYDVLDKRRGRDPEMGGKDTQALLRNDQVYQPLRDRDDAQYSHLGGNKGPRRKNPQEGLYNELQKDKMAEAYSEIGMALPPRGRVKFSRSADERPPPVPNPDYEPIRKGQRDLYSGLNQRRGRDPEMGGKERRRGKGHDGLYQGLSTATKDTYDALHMQKGDKQTLLPNDQLYQPLKDREDDQYSHLQGNALPPR

SEQ ID NO: 31 - protein, 225 aa, synthetic construct
  REGION 1..225: note = Description of sequence: Synthetic polypeptide
RVKFSRSADDTQALLRNDQVYQPLRDRDDAQYSHLGGNRGRDPEMGGKAPAYQQGQNQLYNELNLGRREEYDVLDKRKGERPPPVPNPDYEPIRKGQRDLYSGLNQRALPPRGRVKFSRSADPRRKNPQEGLYNELQKDKMAEAYSEIGMRGRDPEMGGKDKQTLLPNDQLYQPLKDREDDQYSHLQGNKGERRRGKGHDGLYQGLSTATKDTYDALHMQALPPR

SEQ ID NO: 32 - protein, 225 aa, synthetic construct
  REGION 1..225: note = Description of sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 33 - protein, 225 aa, synthetic construct
  REGION 1..225: note = Description of sequence: Synthetic polypeptide
RVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 112 aa, synthetic construct
  REGION 1..112: note = Description of sequence: Synthetic polypeptide
RVKFSRSADAPAYQQGQNQLYAELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 112 aa, synthetic construct
  REGION 1..112: note = Description of sequence: Synthetic polypeptide
RVKFSRSADAPAYQQGQNQLYAELNLGRREEYDVLDKRRGRDPEMGGKPRRKNPQEGLYAELQKDKMAEAYSEIGMKGERRRGKGHDGLYAGLSTATKDTYDALHMQALPPR

SEQ ID NO: 36 - protein, 43 aa, synthetic construct
  REGION 1..43: note = Description of sequence: Synthetic polypeptide
RVKFSRSADAPAYQQGQNQLYAELNLGRREEYDVLDKRALPPR

SEQ ID NO: 37 - protein, 225 aa, synthetic construct
  REGION 1..225: note = Description of sequence: Synthetic polypeptide
RVKFSRSADAPAYQQGQNQLYAELNLGRREEYDVLDKRRGRDPEMGGKDTQALLRNDQVYAPLRDRDDAQYSHLGGNKGPRRKNPQEGLYAELQKDKMAEAYSEIGMALPPRGRVKFSRSADERPPPVPNPDYAPIRKGQRDLYSGLNQRRGRDPEMGGKERRRGKGHDGLYAGLSTATKDTYDALHMQKGDKQTLLPNDQLYAPLKDREDDQYSHLQGNALPPR

SEQ ID NO: 38 - protein, 225 aa, synthetic construct
  REGION 1..225: note = Description of sequence: Synthetic polypeptide
RVKFSRSADDTQALLRNDQVYAPLRDRDDAQYSHLGGNRGRDPEMGGKAPAYQQGQNQLYAELNLGRREEYDVLDKRKGERPPPVPNPDYAPIRKGQRDLYSGLNQRALPPRGRVKFSRSADPRRKNPQEGLYAELQKDKMAEAYSEIGMRGRDPEMGGKDKQTLLPNDQLYAPLKDREDDQYSHLQGNKGERRRGKGHDGLYAGLSTATKDTYDALHMQALPPR

SEQ ID NO: 39 - protein, 233 aa, synthetic construct
  REGION 1..233: note = Description of sequence: Synthetic polypeptide
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 40 - protein, 5 aa, synthetic construct
  REGION 1..5: note = Description of sequence: Synthetic peptide
GGSGG

SEQ ID NO: 41 - protein, 4 aa, synthetic construct
  REGION 1..4: note = Description of sequence: Synthetic peptide
RAKR

SEQ ID NO: 42 - protein, 19 aa, synthetic construct
  REGION 1..19: note = Description of sequence: Synthetic peptide
ATNFSLLKQAGDVEENPGP

SEQ ID NO: 43 - protein, 249 aa, synthetic construct
  REGION 1..249: note = Description of sequence: Synthetic polypeptide
EVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSSGGGGSGGGGSGGGGSGGGGSDIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIK

SEQ ID NO: 44 - protein, 122 aa, synthetic construct
  REGION 1..122: note = Description of sequence: Synthetic polypeptide
EVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSS

SEQ ID NO: 45 - protein, 107 aa, synthetic construct
  REGION 1..107: note = Description of sequence: Synthetic polypeptide
DIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIK

SEQ ID NO: 46 - protein, 248 aa, synthetic construct
  REGION 1..248: note = Description of sequence: Synthetic polypeptide
QLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIK

SEQ ID NO: 47 - protein, 121 aa, synthetic construct
  REGION 1..121: note = Description of sequence: Synthetic polypeptide
QLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSS

SEQ ID NO: 48 - protein, 107 aa, synthetic construct
  REGION 1..107: note = Description of sequence: Synthetic polypeptide
AIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIK

SEQ ID NO: 49 - protein, 254 aa, synthetic construct
  REGION 1..254: note = Description of sequence: Synthetic polypeptide
QVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVL

SEQ ID NO: 50 - protein, 124 aa, synthetic construct
  REGION 1..124: note = Description of sequence: Synthetic polypeptide
QVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSS

SEQ ID NO: 51 - protein, 110 aa, synthetic construct
  REGION 1..110: note = Description of sequence: Synthetic polypeptide
QSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVL

SEQ ID NO: 52 - protein, 5 aa, synthetic construct
  REGION 1..5: note = Description of sequence: Synthetic peptide
GGGGS

SEQ ID NO: 53 - protein, 4 aa, synthetic construct
  REGION 1..4: note = Description of sequence: Synthetic peptide
GGGS

SEQ ID NO: 54 - protein, 32 aa, synthetic construct
  REGION 1..32: note = Description of sequence: Synthetic polypeptide
LYCNHRNRRRVCKCPRPVVKSGDKPSLSARYV

SEQ ID NO: 55 - protein, 8 aa, synthetic construct
  REGION 1..8: note = Description of sequence: Synthetic peptide
RRVCKCPR

SEQ ID NO: 56 - protein, 16 aa, synthetic construct
  REGION 1..16: note = Description of sequence: Synthetic peptide
RRVCKCPRRRVCKCPR

SEQ ID NO: 57 - protein, 9 aa, synthetic construct
  REGION 1..9: note = Description of sequence: Synthetic peptide
RVCKCPRPV

SEQ ID NO: 58 - protein, 15 aa, synthetic construct
  REGION 1..15: note = Description of sequence: Synthetic peptide
YQPYAPPRDFAAYRS

SEQ ID NO: 59 - protein, 15 aa, synthetic construct
  REGION 1..15: note = Description of sequence: Synthetic peptide
FQPFAPPRDFAAFRS

SEQ ID NO: 60 - protein, 9 aa, synthetic construct
  REGION 1..9: note = Description of sequence: Synthetic peptide
RVKFSRSAD

SEQ ID NO: 61 - protein, 10 aa, synthetic construct
  REGION 1..10: note = Description of sequence: Synthetic peptide
RGRDPEMGGK

SEQ ID NO: 62 - skipped

SEQ ID NO: 63 - protein, 5 aa, synthetic construct
  REGION 1..5: note = Description of sequence: Synthetic peptide
ALPPR

SEQ ID NO: 64 - protein, 18 aa, synthetic construct
  REGION 1..18: note = Description of sequence: Synthetic peptide
RMSQIKRLLSEKKTCQCP

SEQ ID NO: 65 - protein, 29 aa, synthetic construct
  REGION 1..29: note = Description of sequence: Synthetic peptide
APAYQQGQNQLYAELNLGRREEYDVLDKR

SEQ ID NO: 66 - protein, 28 aa, synthetic construct
  REGION 1..28: note = Description of sequence: Synthetic peptide
PRRKNPQEGLYAELQKDKMAEAYSEIGM

SEQ ID NO: 67 - protein, 29 aa, synthetic construct
  REGION 1..29: note = Description of sequence: Synthetic peptide
ERRRGKGHDGLYAGLSTATKDTYDALHMQ

SEQ ID NO: 68 - protein, 29 aa, synthetic construct
  REGION 1..29: note = Description of sequence: Synthetic peptide
DTQALLRNDQVYAPLRDRDDAQYSHLGGN

SEQ ID NO: 69 - protein, 28 aa, synthetic construct
  REGION 1..28: note = Description of sequence: Synthetic peptide
ERPPPVPNPDYAPIRKGQRDLYSGLNQR

SEQ ID NO: 70 - protein, 29 aa, synthetic construct
  REGION 1..29: note = Description of sequence: Synthetic peptide
DKQTLLPNDQLYAPLKDREDDQYSHLQGN

SEQ ID NO: 71 - protein, 530 aa, synthetic construct
  REGION 1..530: note = Description of sequence: Synthetic polypeptide
LTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALE

SEQ ID NO: 72 - protein, 530 aa, synthetic construct
  REGION 1..530: note = Description of sequence: Synthetic polypeptide
LTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALE

SEQ ID NO: 73 - protein, 530 aa, synthetic construct
  REGION 1..530: note = Description of sequence: Synthetic polypeptide
LTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALE

SEQ ID NO: 74 - protein, 539 aa, synthetic construct
  REGION 1..539: note = Description of sequence: Synthetic polypeptide
NPQRSTVWYLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGRPALE

SEQ ID NO: 75 - protein, 530 aa, synthetic construct
  REGION 1..530: note = Description of sequence: Synthetic polypeptide
LTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALE

SEQ ID NO: 76 - protein, 530 aa, synthetic construct
  REGION 1..530: note = Description of sequence: Synthetic polypeptide
LTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGRPALE

SEQ ID NO: 77 - protein, 530 aa, synthetic construct
  REGION 1..530: note = Description of sequence: Synthetic polypeptide
LTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALE

SEQ ID NO: 78 - protein, 530 aa, synthetic construct
  REGION 1..530: note = Description of sequence: Synthetic polypeptide
LTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALE

SEQ ID NO: 79 - protein, 136 aa, synthetic construct
  REGION 1..136: note = Description of sequence: Synthetic polypeptide
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 80 - protein, 157 aa, synthetic construct
  REGION 1..157: note = Description of sequence: Synthetic polypeptide
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 81 - protein, 9 aa, synthetic construct
  REGION 1..9: note = Description of sequence: Synthetic peptide
CPYSNPSLC

SEQ ID NO: 82 - protein, 24 aa, synthetic construct
  REGION 1..24: note = Description of sequence: Synthetic peptide
NSELLSLINDMPITNDQKKLMSNN

SEQ ID NO: 83 - protein, 12 aa, synthetic construct
  REGION 1..12: note = Description of sequence: Synthetic peptide
CQFDLSTRRLKC

SEQ ID NO: 84 - protein, 12 aa, synthetic construct
  REGION 1..12: note = Description of sequence: Synthetic peptide
CQYNLSSRALKC

SEQ ID NO: 85 - protein, 12 aa, synthetic construct
  REGION 1..12: note = Description of sequence: Synthetic peptide
CVWQRWQKSYVC

SEQ ID NO: 86 - protein, 12 aa, synthetic construct
  REGION 1..12: note = Description of sequence: Synthetic peptide
CMWDRFSRWYKC

SEQ ID NO: 87 - protein, 25 aa, synthetic construct
  REGION 1..25: note = Description of sequence: Synthetic peptide
SFVLNWYRMSPSNQTDKLAAFPEDR

SEQ ID NO: 88 - protein, 19 aa, synthetic construct
  REGION 1..19: note = Description of sequence: Synthetic peptide
SGTYLCGAISLAPKAQIKE

SEQ ID NO: 89 - protein, 24 aa, synthetic construct
  REGION 1..24: note = Description of sequence: Synthetic peptide
ELPTQGTFSNVSTNVSPAKPTTTA

SEQ ID NO: 90 - protein, 12 aa, synthetic construct
  REGION 1..12: note = Description of sequence: Synthetic peptide
GQNDTSQTSSPS

SEQ ID NO: 91 - protein, 531 aa, synthetic construct
  REGION 1..531: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 560 aa, synthetic construct
  REGION 1..560: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 544 aa, synthetic construct
  REGION 1..544: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITRRVCKCPRRRVCKCPRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 554 aa, synthetic construct
  REGION 1..554: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCGGGSYQPYAPPRDFAAYRSGGGSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 554 aa, synthetic construct
  REGION 1..554: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCGGGSFQPFAPPRDFAAFRSGGGSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 545 aa, synthetic construct
  REGION 1..545: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGGSRVCKCPRPV

SEQ ID NO: 97 - protein, 550 aa, synthetic construct
  REGION 1..550: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGSFQPFAPPRDFAAFRS

SEQ ID NO: 98 - protein, 550 aa, synthetic construct
  REGION 1..550: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGSYQPYAPPRDFAAYRS

SEQ ID NO: 99 - protein, 560 aa, synthetic construct
  REGION 1..560: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELLYCNHRNRRRVCKCPRPVVKSGDKPSLSARYVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 544 aa, synthetic construct
  REGION 1..544: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRRVCKCPRRRVCKCPRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 644 aa, synthetic construct
  REGION 1..644: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKDTQALLRNDQVYQPLRDRDDAQYSHLGGNKGPRRKNPQEGLYNELQKDKMAEAYSEIGMALPPRGRVKFSRSADERPPPVPNPDYEPIRKGQRDLYSGLNQRRGRDPEMGGKERRRGKGHDGLYQGLSTATKDTYDALHMQKGDKQTLLPNDQLYQPLKDREDDQYSHLQGNALPPR

SEQ ID NO: 102 - protein, 644 aa, synthetic construct
  REGION 1..644: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 643 aa, synthetic construct
  REGION 1..643: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYAELNLGRREEYDVLDKRRGRDPEMGGKPRRKNPQEGLYAELQKDKMAEAYSEIGMKGERRRGKGHDGLYAGLSTATKDTYDALHMQALPPRRVKFSRSADAPAYQQGQNQLYAELNLGRREEYDVLDKRRGRDPEMGGKPRRKNPQEGLYAELQKDKMAEAYSEIGMKGERRRGKGHDGLYAGLSTATKDTYDALHMQALPPR

SEQ ID NO: 104 - protein, 644 aa, synthetic construct
  REGION 1..644: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYAELNLGRREEYDVLDKRRGRDPEMGGKDTQALLRNDQVYAPLRDRDDAQYSHLGGNKGPRRKNPQEGLYAELQKDKMAEAYSEIGMALPPRGRVKFSRSADERPPPVPNPDYAPIRKGQRDLYSGLNQRRGRDPEMGGKERRRGKGHDGLYAGLSTATKDTYDALHMQKGDKQTLLPNDQLYAPLKDREDDQYSHLQGNALPPR

SEQ ID NO: 105 - protein, 644 aa, synthetic construct
  REGION 1..644: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYAPLRDRDDAQYSHLGGNRGRDPEMGGKAPAYQQGQNQLYAELNLGRREEYDVLDKRKGERPPPVPNPDYAPIRKGQRDLYSGLNQRALPPRGRVKFSRSADPRRKNPQEGLYAELQKDKMAEAYSEIGMRGRDPEMGGKDKQTLLPNDQLYAPLKDREDDQYSHLQGNKGERRRGKGHDGLYAGLSTATKDTYDALHMQALPPR

SEQ ID NO: 106 - protein, 644 aa, synthetic construct
  REGION 1..644: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYQPLRDRDDAQYSHLGGNRGRDPEMGGKAPAYQQGQNQLYNELNLGRREEYDVLDKRKGERPPPVPNPDYEPIRKGQRDLYSGLNQRALPPRGRVKFSRSADPRRKNPQEGLYNELQKDKMAEAYSEIGMRGRDPEMGGKDKQTLLPNDQLYQPLKDREDDQYSHLQGNKGERRRGKGHDGLYQGLSTATKDTYDALHMQALPPR

SEQ ID NO: 107 - protein, 644 aa, synthetic construct
  REGION 1..644: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 644 aa, synthetic construct
  REGION 1..644: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 109 - protein, 531 aa, synthetic construct
  REGION 1..531: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYAELNLGRREEYDVLDKRRGRDPEMGGKPRRKNPQEGLYAELQKDKMAEAYSEIGMKGERRRGKGHDGLYAGLSTATKDTYDALHMQALPPR

SEQ ID NO: 110 - protein, 531 aa, synthetic construct
  REGION 1..531: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYQPLRDRDDAQYSHLGGNRGRDPEMGGKPRRKNPQEGLYNELQKDKMAEAYSEIGMKGERRRGKGHDGLYQGLSTATKDTYDALHMQALPPR

SEQ ID NO: 111 - protein, 530 aa, synthetic construct
  REGION 1..530: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADERPPPVPNPDYEPIRKGQRDLYSGLNQRRGRDPEMGGKPRRKNPQEGLYNELQKDKMAEAYSEIGMKGERRRGKGHDGLYQGLSTATKDTYDALHMQALPPR

SEQ ID NO: 112 - protein, 531 aa, synthetic construct
  REGION 1..531: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDKQTLLPNDQLYQPLKDREDDQYSHLQGNRGRDPEMGGKPRRKNPQEGLYNELQKDKMAEAYSEIGMKGERRRGKGHDGLYQGLSTATKDTYDALHMQALPPR

SEQ ID NO: 113 - protein, 531 aa, synthetic construct
  REGION 1..531: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 114 - protein, 531 aa, synthetic construct
  REGION 1..531: note = Description of sequence: Synthetic polypeptide
MALPVTALLLPLALLLHAARPGGGGSCPYSNPSLCGGGGSCPYSNPSLCGGGGSQVQLQQSGPGLVKPSQTLSLTCAISGDSVSSNSATWNWIRQSPSRGLEWLGRTYYRSKWYDDYAVSVKSRITINPDTSKNHLSLHLNSVTPEDTAVYYCAGGGLVGAPDGFDVWGQGTMVTVSSGGGGSGGGGSGGGGSGGGGSQSVLTQPPSASGTPGQRVTISCSGSSSNIGSDPVNWYQQLPGTAPKLLIYSNNQRPSGVPDRFSGSKSGTSASLAISGLQSEDEADYYCSAWDDSLNGYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYAELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 530 aa, synthetic construct
  REGION 1..530: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 559 aa, synthetic construct
  REGION 1..559: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 543 aa, synthetic construct
  REGION 1..543: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITRRVCKCPRRRVCKCPRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 553 aa, synthetic construct
  REGION 1..553: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCGGGSYQPYAPPRDFAAYRSGGGSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 553 aa, synthetic construct
  REGION 1..553: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCGGGSFQPFAPPRDFAAFRSGGGSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 544 aa, synthetic construct
  REGION 1..544: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGGSRVCKCPRPV

SEQ ID NO: 121 - protein, 549 aa, synthetic construct
  REGION 1..549: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGSFQPFAPPRDFAAFRS

SEQ ID NO: 122 - protein, 549 aa, synthetic construct
  REGION 1..549: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGSYQPYAPPRDFAAYRS

SEQ ID NO: 123 - protein, 559 aa, synthetic construct
  REGION 1..559: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELLYCNHRNRRRVCKCPRPVVKSGDKPSLSARYVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 543 aa, synthetic construct
  REGION 1..543: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRRVCKCPRRRVCKCPRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 643 aa, synthetic construct
  REGION 1..643: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 642 aa, synthetic construct
  REGION 1..642: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYAELNLGRREEYDVLDKRRGRDPEMGGKPRRKNPQEGLYAELQKDKMAEAYSEIGMKGERRRGKGHDGLYAGLSTATKDTYDALHMQALPPRRVKFSRSADAPAYQQGQNQLYAELNLGRREEYDVLDKRRGRDPEMGGKPRRKNPQEGLYAELQKDKMAEAYSEIGMKGERRRGKGHDGLYAGLSTATKDTYDALHMQALPPR

SEQ ID NO: 127 - protein, 643 aa, synthetic construct
  REGION 1..643: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYAELNLGRREEYDVLDKRRGRDPEMGGKDTQALLRNDQVYAPLRDRDDAQYSHLGGNKGPRRKNPQEGLYAELQKDKMAEAYSEIGMALPPRGRVKFSRSADERPPPVPNPDYAPIRKGQRDLYSGLNQRRGRDPEMGGKERRRGKGHDGLYAGLSTATKDTYDALHMQKGDKQTLLPNDQLYAPLKDREDDQYSHLQGNALPPR

SEQ ID NO: 128 - protein, 643 aa, synthetic construct
  REGION 1..643: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKDTQALLRNDQVYQPLRDRDDAQYSHLGGNKGPRRKNPQEGLYNELQKDKMAEAYSEIGMALPPRGRVKFSRSADERPPPVPNPDYEPIRKGQRDLYSGLNQRRGRDPEMGGKERRRGKGHDGLYQGLSTATKDTYDALHMQKGDKQTLLPNDQLYQPLKDREDDQYSHLQGNALPPR

SEQ ID NO: 129 - protein, 643 aa, synthetic construct
  REGION 1..643: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYAPLRDRDDAQYSHLGGNRGRDPEMGGKAPAYQQGQNQLYAELNLGRREEYDVLDKRKGERPPPVPNPDYAPIRKGQRDLYSGLNQRALPPRGRVKFSRSADPRRKNPQEGLYAELQKDKMAEAYSEIGMRGRDPEMGGKDKQTLLPNDQLYAPLKDREDDQYSHLQGNKGERRRGKGHDGLYAGLSTATKDTYDALHMQALPPR

SEQ ID NO: 130 - protein, 643 aa, synthetic construct
  REGION 1..643: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYQPLRDRDDAQYSHLGGNRGRDPEMGGKAPAYQQGQNQLYNELNLGRREEYDVLDKRKGERPPPVPNPDYEPIRKGQRDLYSGLNQRALPPRGRVKFSRSADPRRKNPQEGLYNELQKDKMAEAYSEIGMRGRDPEMGGKDKQTLLPNDQLYQPLKDREDDQYSHLQGNKGERRRGKGHDGLYQGLSTATKDTYDALHMQALPPR

SEQ ID NO: 131 - protein, 643 aa, synthetic construct
  REGION 1..643: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 643 aa, synthetic construct
  REGION 1..643: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 133 - protein, 530 aa, synthetic construct
  REGION 1..530: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYAELNLGRREEYDVLDKRRGRDPEMGGKPRRKNPQEGLYAELQKDKMAEAYSEIGMKGERRRGKGHDGLYAGLSTATKDTYDALHMQALPPR

SEQ ID NO: 134 - protein, 530 aa, synthetic construct
  REGION 1..530: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYQPLRDRDDAQYSHLGGNRGRDPEMGGKPRRKNPQEGLYNELQKDKMAEAYSEIGMKGERRRGKGHDGLYQGLSTATKDTYDALHMQALPPR

SEQ ID NO: 135 - protein, 529 aa, synthetic construct
  REGION 1..529: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADERPPPVPNPDYEPIRKGQRDLYSGLNQRRGRDPEMGGKPRRKNPQEGLYNELQKDKMAEAYSEIGMKGERRRGKGHDGLYQGLSTATKDTYDALHMQALPPR

SEQ ID NO: 136 - protein, 530 aa, synthetic construct
  REGION 1..530: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDKQTLLPNDQLYQPLKDREDDQYSHLQGNRGRDPEMGGKPRRKNPQEGLYNELQKDKMAEAYSEIGMKGERRRGKGHDGLYQGLSTATKDTYDALHMQALPPR

SEQ ID NO: 137 - protein, 530 aa, synthetic construct
  REGION 1..530: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 138 - protein, 530 aa, synthetic construct
  REGION 1..530: note = Description of sequence: Synthetic polypeptide
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYAELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 522 aa, synthetic construct
  REGION 1..522: note = Description of sequence: Synthetic polypeptide
MALPVTALLLPLALLLHAARPEVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSSGGGGSGGGGSGGGGSGGGGSDIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIKTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 506 aa, synthetic construct
  REGION 1..506: note = Description of sequence: Synthetic polypeptide
MALPVTALLLPLALLLHAARPEVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSSGGGGSGGGGSGGGGSGGGGSDIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIKTTTPAPRPPTPAPTIASQPLSLRPEACRPAAGGAVHTRGLDFACDIYIWAPLAGTCGVLLLSLVITRRVCKCPRRRVCKCPRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 516 aa, synthetic construct
  REGION 1..516: note = Description of sequence: Synthetic polypeptide
MALPVTALLLPLALLLHAARPEVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSSGGGGSGGGGSGGGGSGGGGSDIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIKTTTPAPRPPTPAPTIASQPLSLRPEACRPAAGGAVHTRGLDFACDIYIWAPLAGTCGVLLLSLVITLYCGGGSYQPYAPPRDFAAYRSGGGSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 516 aa, synthetic construct
  REGION 1..516: note = Description of sequence: Synthetic polypeptide
MALPVTALLLPLALLLHAARPEVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSSGGGGSGGGGSGGGGSGGGGSDIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIKTTTPAPRPPTPAPTIASQPLSLRPEACRPAAGGAVHTRGLDFACDIYIWAPLAGTCGVLLLSLVITLYCGGGSFQPFAPPRDFAAFRSGGGSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 507 aa, synthetic construct
  REGION 1..507: note = Description of sequence: Synthetic polypeptide
MALPVTALLLPLALLLHAARPEVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSSGGGGSGGGGSGGGGSGGGGSDIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGGSRVCKCPRPV

SEQ ID NO: 144 - protein, 512 aa, synthetic construct
  REGION 1..512: note = Description of sequence: Synthetic polypeptide
MALPVTALLLPLALLLHAARPEVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSSGGGGSGGGGSGGGGSGGGGSDIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGSFQPFAPPRDFAAFRS

SEQ ID NO: 145 - protein, 522 aa, synthetic construct
  REGION 1..522: note = Description of sequence: Synthetic polypeptide
MALPVTALLLPLALLLHAARPEVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSSGGGGSGGGGSGGGGSGGGGSDIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIKTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELLYCNHRNRRRVCKCPRPVVKSGDKPSLSARYVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 506 aa, synthetic construct
  REGION 1..506: note = Description of sequence: Synthetic polypeptide
MALPVTALLLPLALLLHAARPEVQLLESGGGLVQPGGSLRLSCAASGFTFSSYAMNWVRQAPGKGLEWVSTISGSGGSTYYADSVKGRFTISRDNSKNTLYLQMNSLRAEDTAVFYCAIDPEYYDILTGGDYWGQGTLVTVSSGGGGSGGGGSGGGGSGGGGSDIQMTQSPSAMSASVGDRVTITCRASQGISNYLAWFQQKPGKVPKRLIYAASSLQSGVPSRFSGSGSGTEFTLTISSLQPEDFATYFCLQHDSFPLTFGGGTKVEIKTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRRVCKCPRRRVCKCPRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 15 aa, synthetic construct
  REGION 1..15: note = Description of sequence: Synthetic peptide
GGGGSGGGGSGGGGS

SEQ ID NO: 148 - protein, 5 aa, synthetic construct
  REGION 1..5: note = Description of sequence: Synthetic peptide
SGGGG

SEQ ID NO: 149 - protein, 6 aa, synthetic construct
  REGION 1..6: note = Description of sequence: Synthetic peptide
SGGGGS

SEQ ID NO: 150 - protein, 21 aa, synthetic construct
  REGION 1..21: note = Description of sequence: Synthetic peptide
MALPVTALLLPLALLLHAARP

SEQ ID NO: 151 - protein, 18 aa, synthetic construct
  REGION 1..18: note = Description of sequence: Synthetic peptide
MIPAVVLLLLLLVEQAAA

SEQ ID NO: 152 - protein, 4 aa, synthetic construct
  REGION 1..4: note = Description of sequence: Synthetic peptide
YAEL

SEQ ID NO: 153 - protein, 4 aa, synthetic construct
  REGION 1..4: note = Description of sequence: Synthetic peptide
YAGL

SEQ ID NO: 154 - protein, 4 aa, synthetic construct
  REGION 1..4: note = Description of sequence: Synthetic peptide
YAPL

SEQ ID NO: 155 - protein, 4 aa, synthetic construct
  REGION 1..4: note = Description of sequence: Synthetic peptide
YAPI

SEQ ID NO: 156 - protein, 4 aa, synthetic construct
  REGION 1..4: note = Description of sequence: Synthetic peptide
YNEL

SEQ ID NO: 157 - protein, 4 aa, synthetic construct
  REGION 1..4: note = Description of sequence: Synthetic peptide
YNGL

(19) United States
(12) Patent Application Publication
(10) Pub. No.: US 2025/0230410 A1
(43) Pub. Date: Jul. 17, 2025

(54) CHIMERIC ANTIGEN RECEPTORS WITH ENHANCED SIGNALING AND ACTIVITIES AND USES THEREOF

(71) Applicant: Allogene Therapeutics, Inc., South San Francisco, CA (US)

(72) Inventors: Michael Thomas BETHUNE, Castro Valley, CA (US); Yi ZHANG, Foster City, CA (US); Thomas John VAN BLARCOM, Oakland, CA (US); Siler PANOWSKI, Barkeley, CA (US); Barbra Johnson SASU, San Francisco, CA (US)

(21) Appl. No.: 18/987,766

(22) Filed: Dec. 19, 2024

Related U.S. Application Data
(62) Division of application No. 17/381,693, filed on Jul. 21, 2021, now Pat. No. 12,215,347.
(60) Provisional application No. 63/219,710, filed on Jul. 8, 2021.
(60) Provisional application No. 63/054,701, filed on Jul. 21, 2020.

(51) Int. Cl. C12N 5/0783; A61K 40/11; A61K 40/31; A61K 40/42; C07K 14/725; C12N 15/86

(52) U.S. Cl. CPC C12N 5/0636; A61K 40/11; A61K 40/31; A61K 40/4202; C07K 14/7051; C12N 15/86; A61K 2239/22

(57) ABSTRACT

Provided herein are recombinant antigen receptors, for example chimeric antigen receptors (CARs), that comprise modified cytoplasmic domains that provide improved signalling and thereby provide improved performance and safety. Also provided are polynucleotides encoding the recombinant antigen receptors, vectors comprising the polynucleotides, and engineered immune cells comprising the vectors and/or polynucleotides. The invention further provides methods for engineering immune cells to express the recombinant antigen receptors. Improved recombinant antigen receptor signalling is also provided by co-expressing a first recombinant antigen receptor and a second recombinant antigen receptor or co-expressing a recombinant antigen receptor and a protein involved in transducing the signal from the activated recombinant antigen receptor. Also provided are methods of treating a variety of conditions, including, but not limited to, blood cancers and cancers characterized by solid tumors, by administering the engineered cells to patients suffering from such a condition.

### CROSS REFERENCE TO RELATED APPLICATIONS

The present application claims the benefit of priority to U.S. application Ser. No. 17/381,693, filed on Jul. 21, 2021, which claims the benefit of priority to U.S. Provisional Application No. 63/054,701, filed on Jul. 21, 2020; and U.S. Provisional Application No. 63/219,710, filed on Jul. 8, 2021, the contents of all of which are hereby incorporated by reference in their entireties.

### SEQUENCE LISTING

The instant application contains a Sequence Listing which has been submitted electronically in XML format and is hereby incorporated by reference in its entirety. Said XML file, created on Dec. 2, 2024, is named AT-039_ST26.xml and is 242,225 bytes in size.

### FIELD

The invention relates to improvements in signalling by recombinant antigen receptors such as chimeric antigen receptors. The improvements include modifications to the intracellular signaling domain of the recombinant antigen receptors and other methods for enhancing signal transduction when the recombinant antigen receptor is activated by ligand or antigen binding. The invention also relates to the incorporation of these improvements into engineered immune cells and the use of such cells to treat cancer and other maladies.

### BACKGROUND

Adoptive transfer of immune cells genetically modified to recognize malignancy-associated antigens is showing promise as a new approach to treating cancer (see, e.g., Brenner et al., Current Opinion in Immunology, 22(2): 251-257 (2010); Rosenberg et al., Nature Reviews Cancer, 8(4): 299-308 (2008)). T cells can be genetically modified to express chimeric antigen receptors (CARs), fusion proteins comprised of an antigen recognition moiety and T cell activation domains (see, e.g., Eshhar et al., Proc. Natl. Acad. Sci. USA, 90(2): 720-724 (1993), and Sadelain et al., Curr. Opin. Immunol, 21(2): 215-223 (2009)).

While CARs present various therapeutic advantages, they lack some of the complexities that contribute to the sensitivity, specificity and strength of the natural interaction between T cells and their targets. For example, the co-receptor (CD4 or CD8)-associated Lck kinase that is central to TCR signaling is coordinated with target recognition for the TCR synapse but is not for the CAR synapse (FIG. 2A). See, e.g., Davenport, A. J. et al. Chimeric antigen receptor T cells form nonclassical and potent immune synapses driving rapid cytotoxicity. Proc. Natl. Acad. Sci. U.S.A 115, E2068-E2076 (2018). Further, a CD3 zeta CAR has only 3 ITAMs, all of which derive from CD3 zeta, whereas the TCR/CD3 holocomplex has 10 ITAMs comprising contributions from all four CD3 chains (delta, epsilon, gamma, and zeta). Reducing the number or diversity of ITAMs modulates both TCR and CAR function. See, e.g., Bettini, M. L. et al. Cutting Edge: CD3 ITAM Diversity Is Required for Optimal TCR Signaling and Thymocyte Development. J. Immunol. 199, 1555-1560 (2017); Feucht, J. et al. Calibration of CAR activation potential directs alternative T cell fates and therapeutic potency. Nature Medicine 25, 82-88 (2019). Thus, the quantity and quality of ITAMs and the complement of proximate signaling mediators differ between CARs and TCRs. Perhaps resultingly, TCRs are more sensitive to low-density antigens than are CARs, with the former mediating killing of target cells expressing as few as three cognate peptide-MHC complexes. See, e.g., Purbhoo, M. A., Irvine, D. J., Huppa, J. B. & Davis, M. M. T cell killing does not require the formation of a stable mature immunological synapse. Nat. Immunol. 5, 524-530 (2004).

Accordingly, there is a need for improvements in CAR-based therapy. Provided herein are methods and compositions that address this need.

### SUMMARY

In one aspect, the present invention provides a recombinant antigen receptor comprising an extracellular antigen binding domain, a transmembrane domain, and an intracellular domain that comprises a co-stimulatory domain and an ITAM-containing domain, wherein
(a) the ITAM-containing domain comprises one, two or three or more ITAM domains selected from the group consisting of a wildtype CD3g ITAM, CD3d ITAM, CD3e ITAM, CD3z1 ITAM, CD3z2 ITAM, CD3z3 ITAM, and a variant thereof, and wherein if the ITAM-containing domain comprises only three wildtype ITAM domains, the three ITAMs domains are not CD3z1, CD3z2 and CD3z3; and/or
(b) the intracellular domain further comprises a Lck recruiting motif (LRM).

In an embodiment of the recombinant antigen receptor described herein, the recombinant antigen receptor comprises a chimeric antigen receptor (CAR). In an embodiment of the recombinant antigen receptor described herein, the antigen binding domain comprises a heavy chain variable domain (VH) and a light chain variable domain (VL). In any of the recombinant antigen receptor embodiments disclosed herein, the variant ITAM domain may comprise an Ala substitution at the second position in the canonical YXX(L/I) ITAM motif of a wildtype CD3z1, CD3z2, CD3z3, CD3d, CD3e, or CD3g. In an embodiment, the co-stimulatory domain comprises 4-1BB co-stimulatory domain. In an embodiment, the number of ITAM domains is a multiple of three. For example, the ITAM-containing domain may consist of three ITAM domains or six ITAM domains. In an embodiment, the ITAM-containing domain comprises from N-terminus to C-terminus:
(a) CD3d ITAM, CD3z2 ITAM, CD3z3 ITAM;
(b) CD3e ITAM, CD3z2 ITAM, CD3z3 ITAM;
(c) CD3g ITAM, CD3z2 ITAM, CD3z3 ITAM;
(d) CD3d ITAM, CD3e ITAM, CD3g ITAM;
(e) CD3z1 ITAM, CD3z2 ITAM, CD3z3 ITAM, CD3z1 ITAM, CD3z2 ITAM, CD3z3 ITAM;
(f) CD3z1 ITAM, CD3d ITAM, CD3z2 ITAM, CD3e ITAM, CD3z3 ITAM, CD3g ITAM;
(g) CD3d ITAM, CD3z1 ITAM, CD3e ITAM, CD3z2 ITAM, CD3g ITAM, CD3z3 ITAM;
(h) CD3z1 ITAM, CD3z2 ITAM, CD3z3 ITAM, CD3d ITAM, CD3e ITAM, CD3g ITAM;
(i) CD3d ITAM, CD3e ITAM, CD3g ITAM, CD3z1 ITAM, CD3z2 ITAM, CD3z3 ITAM;
(j) CD3z1 (YAEL (SEQ ID NO: 152)) ITAM, CD3z2 ITAM, CD3z3 ITAM;
(k) CD3z1 (YAEL (SEQ ID NO: 152)) ITAM, CD3z2 (YAEL (SEQ ID NO: 152)) ITAM, CD3z3 (YAGL (SEQ ID NO: 153)) ITAM;
(l) CD3z1 (YAEL (SEQ ID NO: 152));
(m) CD3z1 (YAEL (SEQ ID NO: 152)) ITAM, CD3d (YAPL (SEQ ID NO: 154)) ITAM, CD3z2 (YAEL (SEQ ID NO: 152)) ITAM, CD3e (YAPI (SEQ ID NO: 155)) ITAM, CD3z3 (YAGL (SEQ ID NO: 153)) ITAM, CD3g (YAPL (SEQ ID NO: 154)) ITAM; or
(n) CD3d (YAPL (SEQ ID NO: 154)) ITAM, CD3z1 (YAEL (SEQ ID NO: 152)) ITAM, CD3e (YAPI (SEQ ID NO: 155)) ITAM, CD3z2 (YAEL (SEQ ID NO: 152)) ITAM, CD3g (YAPL (SEQ ID NO: 154)) ITAM, CD3z3 (YAGL (SEQ ID NO: 153)) ITAM.

In certain embodiments, the ITAM-containing domain is derived from or based on a CD3z ITAM-containing domain framework.

In certain embodiments, the ITAM-containing domain comprises an ITAM domain comprising the amino acid sequence selected from SEQ ID NOs: 20-25, 65-70. In certain embodiments, the ITAM-containing domain comprises an amino acid sequence selected from SEQ ID NOs: 26-38.

In an embodiment, the LRM comprises an LRM of CD4, CD8 or CD28. In an embodiment, the LRM comprises a CD4LRM, CD8 LRM-1, 2×CD8LRM-1, CD8LRM-2, CD28LRM or a CD28LRMY3, or comprises an LRM derived from one of AXL, CD2, CD5, CD44, CD45, and CD122. In an embodiment, the LRM comprises an amino acid sequence selected from the group consisting of SEQ ID NOs: 55, 56, 57, 58, 59, and 64.

In an embodiment, the recombinant antigen receptor comprises an intracellular domain that comprises an amino acid sequence selected from the group consisting of SEQ ID NOs:26-38, SEQ ID NOs: 55-59, and SEQ ID NOs: 64-70. In an embodiment, the recombinant antigen receptor comprises an amino acid sequence selected from the group consisting of SEQ ID NOs:26-38, SEQ ID NOs: 55-59, and SEQ ID NOs: 64-70. In an embodiment, the recombinant antigen receptor comprises the amino acid sequence of one or more of SEQ ID NOs:26-38, SEQ ID NOs: 55-59, and SEQ ID NOs: 64-70. In an embodiment, the recombinant antigen receptor comprises the amino acid sequence of one or more of SEQ ID NOs: 30-35, 37-38, 57 and 59. In an embodiment, the recombinant antigen receptor comprises the amino acid sequence of one or more of SEQ ID NOs: 30, 35, 37, 57 and 59.

In certain embodiments, the recombinant antigen receptor binds to an antigen expressed on a tumor cell. In certain embodiments, the antigen is expressed at a low density on the tumor cell. In certain embodiments, the low-density antigen is expressed at or below about 10,000 copies per cell. In certain embodiments, the antigen is expressed at a low level naturally. In some embodiments, the expression of the antigen is reduced after the cells are exposed to CAR T treatment. In an embodiment, the recombinant antigen receptor binds to DLL3. In another embodiment, the recombinant antigen receptor binds to CD19. In an embodiment, the recombinant antigen receptor binds to BCMA.

In another aspect, the present invention provides a polynucleotide comprising a DNA sequence encoding the recombinant antigen receptor as described herein.

In another aspect, the present invention provides a vector comprising a herein-described polynucleotide.

In another aspect, the present invention provides an engineered immune cell comprising any one or more of the recombinant antigen receptors described herein. In an embodiment, one or more of the recombinant antigen receptors binds to DLL3.

In an embodiment, the present invention provides an engineered immune cell comprising a recombinant antigen receptor described herein. In an embodiment, the present invention provides an engineered immune cell comprising a recombinant antigen receptor described herein wherein the recombinant antigen receptor binds to DLL3.

In an embodiment, the antigen binding domain binds to DLL3 and comprises a heavy chain variable domain (VH) and a light chain variable domain (VL), and wherein (1) the VH comprises the amino acid sequence of SEQ ID NO: 44 and the VL comprises the amino acid sequence of SEQ ID NO: 45, or (2) the VH comprises the amino acid sequence of SEQ ID NO: 47 and the VL comprises the amino acid sequence of SEQ ID NO: 48, or (3) the VH comprises the amino acid sequence of SEQ ID NO: 50 and the VL comprises the amino acid sequence of SEQ ID NO: 51.

In an embodiment, the recombinant antigen receptor comprises the amino acid sequence selected from SEQ ID NOs: 91-146, with or without a signal peptide.

In an embodiment, the present invention provides an engineered immune cell comprising a first recombinant antigen receptor as described herein and further comprises a second recombinant antigen receptor comprising an extracellular antigen binding domain and an intracellular domain that comprises Lck, LAT, ZAP70, or a variant thereof, e.g. a functional variant thereof. In an embodiment, the first recombinant antigen receptor comprises an amino acid sequence selected from SEQ ID NOs: 26-38, SEQ ID NOs: 55-59, and SEQ ID NOs: 64-70. In an embodiment, the first recombinant antigen receptor comprises an amino acid sequence selected from SEQ ID NOs: 43, 46, 49, and 91-146. In an embodiment, the intracellular domain of the second recombinant antigen receptor comprises the amino acid sequence of one or more of SEQ ID NOs: 1, 2, 3, and 8. In an embodiment, the antigen binding domain of the first recombinant antigen receptor binds to DLL3. In an embodiment of either of the above, either the antigen binding domain of the second recombinant antigen receptor binds to the same antigen as the first recombinant antigen receptor (for example, DLL3), or the antigen binding domain of the second recombinant antigen receptor binds to an antigen different from the antigen that the first recombinant antigen receptor binds to (for example, DLL3).

In another aspect, the present invention provides an engineered immune cell comprising a recombinant antigen receptor, for example, a CAR, and expressing an exogenous downstream mediator of T cell signaling. In an embodiment, the exogenous downstream mediator of T cell signaling comprises the amino acid sequence of ZAP70, Lck, Fyn, Syk, LAT or UNC119 (e.g. the amino acid sequence of SEQ ID NOs: 1, 2, 4, 6, 8 or 9, respectively), or the amino acid sequence of a variant thereof, e.g. a functional variant thereof, for example the amino acid sequence of truncated Lck, truncated Fyn, or of truncated Syk (e.g. the amino acid sequence of SEQ ID NOs: 3, 5 or 7, respectively). In another embodiment, the exogenous downstream mediator of T cell signaling is ZAP70, Lck, Fyn, Syk, LAT, or UNC119 or a variant thereof, e.g. a functional variant thereof, such as truncated Lck, truncated Fyn, or truncated Syk. In an embodiment, the CAR comprises an amino acid sequence selected from SEQ ID NOs: 26-38, SEQ ID NOs: 55-59, and SEQ ID NOs: 64-70. In an embodiment, the CAR comprises an antigen binding domain that binds to DLL3. In an embodiment, the CAR comprises an amino acid sequence selected from SEQ ID NOs: 43, 46, 49, and 91-146.

In another aspect, the invention provides an engineered immune cell comprising a first recombinant antigen receptor and a second recombinant antigen receptor, wherein the first recombinant antigen receptor comprises a CAR and the second recombinant receptor comprises an extracellular antigen binding domain and an intracellular domain that comprises Lck, LAT, Fyn, Syk, UNC119, or ZAP70, or a variant thereof, e.g. a functional variant thereof. In an embodiment, the intracellular domain of the second recombinant antigen receptor comprises the amino acid sequence of one or more of SEQ ID NOs:1-9. In an embodiment, the intracellular domain of the second recombinant antigen receptor comprises the amino acid sequence of one or more of SEQ ID NOs: 1, 2, 3, and 8. In an embodiment, the first recombinant antigen receptor comprises an amino acid sequence selected from SEQ ID NOs: 26-38, SEQ ID NOs: 55-59, and SEQ ID NOs: 64-70. In an embodiment, the antigen binding domain of the first recombinant antigen receptor binds to DLL3. In an embodiment of any of the above, the antigen binding domain of the second recombinant antigen receptor binds to the same antigen as the first recombinant antigen receptor (e.g. DLL3), or the antigen binding domain of the second recombinant antigen receptor binds to an antigen different from the antigen that the first recombinant antigen receptor binds to (e.g. DLL3). In an embodiment, the first recombinant antigen receptor comprises an amino acid sequence selected from SEQ ID NOs: 43, 46, 49, and 91-146.

In various embodiments, any of the engineered immune cells described above is a T cell. In an embodiment, the T cell comprises one or more genomic modifications, such as a genomic modification to the TCRa gene.

In an embodiment, the present invention provides an engineered immune cell that comprises a first recombinant antigen receptor as described herein and further comprises a second recombinant antigen receptor comprising an extracellular antigen binding domain and an intracellular domain that comprises Lck, LAT, Fyn, Syk, UNC119, or ZAP70, or a variant thereof, e.g. a functional variant thereof, and the antigen binding domain of the first recombinant antigen receptor and the antigen binding domain of the second recombinant antigen receptor both bind to DLL3. In an embodiment, the intracellular domain of the second recombinant antigen receptor comprises the amino acid sequence of one or more of SEQ ID NOs:1-9. In an embodiment, the intracellular domain of the second recombinant antigen receptor comprises the amino acid sequence of one or more of SEQ ID NOs: 1, 2, 3, and 8.

In an embodiment, the present invention provides an engineered immune cell that comprises a first recombinant antigen receptor as described herein and further comprises a second recombinant antigen receptor comprising an extracellular antigen binding domain and an intracellular domain that comprises Lck, LAT, Fyn, Syk, UNC119, or ZAP70, or a variant thereof, e.g. a functional variant thereof, and the antigen binding domain of the first recombinant antigen receptor binds to DLL3 and the antigen binding domain of the second recombinant antigen receptor binds to an antigen other than DLL3 and does not bind to DLL3. In an embodiment, the intracellular domain of the second recombinant antigen receptor comprises the amino acid sequence of one or more of SEQ ID NOs:1-9. In an embodiment, the intracellular domain of the second recombinant antigen receptor comprises the amino acid sequence of one or more of SEQ ID NOs: 1, 2, 3, and 8.

In another aspect, the present invention provides a pharmaceutical composition comprising any of the engineered immune cells described herein.

In another aspect, the present invention provides a method of treating cancer comprising administering to a patient in need thereof an effective amount of any of the engineered immune cells described herein and/or a pharmaceutical composition described herein.

In another aspect, the present invention provides a method of making any of the engineered immune cells described herein comprising introducing into an immune cell a polynucleotide that encodes a recombinant antigen receptor and/or CAR described herein, and/or a vector comprising such a polynucleotide, optionally together with another polynucleotide that encodes an exogenous downstream mediator or any of the second recombinant antigen receptors described herein and/or a vector comprising such a polynucleotide. In an embodiment, any one of the polynucleotides or vectors can encode one or more of a recombinant antigen receptor, CAR, exogenous downstream mediator, and/or second recombinant antigen receptors described herein. In an embodiment, the immune cell is a T cell. In an embodiment, the immune cell is an NK cell.

### BRIEF DESCRIPTION OF THE DRAWINGS

FIGS. 1A-1C show altering the quality and quantity of CD3 ITAMs to improve CAR signaling. FIG. 1A shows multiple CAR constructs that contain a different number and/or sequence of CD3 ITAMs selected from γ (gamma), δ (delta), ε (epsilon), and ζ1, ζ2, and ζ3 (zeta 1-3) ITAMs. FIG. 1B shows a CAR construct comprising the CD3zeta wildtype ITAM-containing domain and multiple CAR constructs that have mutations in the canonical ITAM YXXL sequence. FIG. 1B discloses SEQ ID NOS 156, 156-157, 152, 156-157, 152, 152-153 and 152, respectively, in order of appearance. FIG. 1C shows constructs that combine the strategies from FIGS. 1A and 1B (first construct comprises ITAM domains ζ1-YA, δ-YA, ζ2-YA, ε-YA, ζ3-YA, γ-YA; second construct comprises ITAM domains δ-YA, ζ1-YA, ε-YA, ζ2-YA, γ-YA, ζ3-YA). FIG. 1C discloses SEQ ID NOS 152, 154, 152, 155, 153-154, 154, 152, 155, 152, 154 and 153, respectively, in order of appearance.

FIGS. 2A-2G show altering the quality and quantity of CD3ζ ITAMs improves CAR T function in a short-term kinetic killing assay with tumor cells expressing high or low antigen density. FIG. 2A-top shows schematic diagram of expression construct used in the experiments reported in FIGS. 2A-bottom panels and FIG. 2B to FIG. 2G; FIG. 2A, bottom panels are a series of plots (BFP on vertical axis, sDLL3 on horizontal axis) showing the detection of conventional or modified DLL3 CAR expressed on primary human T cells. sDLL3, soluble DLL3. The DLL3 CAR T cells shown in FIG. 2A-bottom panels were co-incubated in a cell killing assay with high antigen density WM266.4 targets at an effector:target ratio of 1:3 (FIG. 2B), or high antigen density WM266.4 targets at an effector:target ratio of 1:1 (FIG. 2C), or low antigen density DMS273 targets at an effector:target ratio of 3:1 (FIG. 2D). The number of target cells was counted every 6 hours for a total of 180 hours. FIG. 2E shows a series of plots showing the detection of conventional or modified DLL3 CAR expressed on primary human T cells from a donor different from the donor for the CAR T cell data shown in the FIG. 2A-bottom panels. The DLL3 CAR T cells shown in FIG. 2E were co-incubated with low antigen density DMS273 targets at an effector:target ratio of 3:1 (FIG. 2F), or high antigen density WM266.4 targets at an effector:target ratio of 1:3 (FIG. 2G). The number of target cells was counted every 6 hours for a total of 120 hours.

FIGS. 3A-3B show that altering the quality and quantity of CD3ζ ITAMs improved CAR T function in a long-term cytotoxicity assay. Primary human T cells were modified to express a conventional or modified CAR, in some cases co-expressing an indicated T cell signaling component. These CAR T effector cells were co-incubated for 19 days with either low antigen density DMS273 tumor targets at an effector:target ratio of 3:1 (FIG. 3A), or high antigen density WM266.4 tumor targets at an effector:target ratio of 1:1 (FIG. 3B). Every 2-3 days, the viability of the luciferase-expressing target cells was measured by luminescence and half of the effectors were transferred to fresh target cells to continue killing. CDζ inactive, all 6 Tyr residues in the 3 ITAMs of CD3ζ were mutated to Phe. NTD, untransduced T cells. % viability was determined based on baselining luminescence data to “No T cells” wells (only target cells added to wells).

FIGS. 4A-4B are schematic representations that show insertion of Lck recruitment motifs (LRM) as a strategy to enable recruitment of Lck to CAR synapses in a more TCR-like manner. The left diagram of FIG. 4A is a schematic representation showing binding of both the CD8 co-receptor (CD4 co-receptor can function the same way) and the TCR to the target cell MHC results in a convergence of CD8-associated Lck and CAR ITAMs, resulting in more efficient signaling. The right diagram of FIG. 4A illustrates that this convergence does not occur in CAR synapses due to the lack of involvement of co-receptors in CAR-antigen binding. As a result, the CAR signaling may be insensitive or ineffective to low density target. FIG. 4B is a schematic representation showing addition of a binding motif sequence for Lck (identified as “LRM”) (other signaling enzymes or adaptors may be used in place of the LRM shown) to the CAR intracellular domain may improve efficiency of CAR signaling due to the directed inclusion of Lck in the CAR synapse.

FIGS. 5A-5D show adding Lck recruitment motif (LRM) to the conventional CAR construct may improve CAR T function as demonstrated in a short-term kinetic killing assay. FIG. 5A is a schematic representation showing LRMs can be inserted at different positions of a conventional CAR construct. FIG. 5B are a series of plots (BFP on vertical axis, sDLL3 on horizontal axis) showing the detection of DLL3 CARs with LRMs inserted between the transmembrane domain and 4-1BB co-stimulation domain (FL: full-length). FIG. 5C shows modified DLL3 CARs depicted in FIG. 5B did not perform as well as the DLL3 CAR without the LRM (CD3ζWT). FIG. 5D shows DLL3 CARs with LRMs at the C-terminus of the construct have better cytotoxicity against low antigen density DMS273 targets (effector:target ratio of 3:1) and high antigen density WM266.4 targets (effector:target ratio of 1:3) than the DLL3 CAR without the LRM. In FIG. 5E, DLL3 CAR T cells shown in FIG. 5D were produced with a different T cell donor. An additional construct having CD28LRMY3 instead of CD8LRM was also tested. These CARs once again showed better cytotoxicity against DMS273 and WM266.4 targets.

FIGS. 6A-6B are schematic representations showing “LckCARs” as a strategy to boost recruitment of Lck to the CAR synapse and to enable combinatorial CAR targeting. FIG. 6A is a schematic representation mimicking the natural convergence of TCR and coreceptors on binding to MHC, with a CAR (T1 CAR) and a second “LckCAR” that bind to the same target on a tumor cell to improve Lck recruitment to the CAR synapse and thus CAR T performance. FIG. 6B is a schematic representation showing targeting the T1 CAR and the “T2 LckCAR” to different targets on a tumor cell to impose the requirement that an otherwise Lck-deficient CAR T cell must engage both targets to achieve functional signaling. This requisite combinatorial targeting (a so-called “AND” logic gate) may enable specific tumor targeting even when one of the targets is present on normal, non-tumor cells.

FIGS. 7A-7B show results of CAR expression and target cell killing assay of CAR T cells expressing the DLL3-specific CAR clone 4H8 with indicated intracellular signaling domains. Results from CAR T cells prepared from two other different donors are shown in FIGS. 8A-8C and FIGS. 9A-9B, respectively.

FIGS. 8D-8E show results of cytokine secretion from CAR T cells expressing DLL3 CARs with various intracellular domains.

FIG. 10 shows results of long-term killing assay of CAR T cells expressing DLL3-specific CAR clone 2G1 cocultured with low density target cell line DMS273 with or without overexpression of PD-L1.

### DETAILED DESCRIPTION

In one aspect, the invention disclosed herein provides improved chimeric antigen receptors (CARs) that have novel cytoplasmic domains that improve CAR T cell performance and engineered immune cells comprising the CARs (e.g. CAR-T cells). The invention also provides polynucleotides encoding these CARs, compositions comprising immune cells expressing these CARs, and methods of making and using these CARs and CAR expressing immune cells. The invention also provides methods for treating a subject having a condition that is treatable using CAR T technology such as cancer, including conditions associated with DLL3, such as small cell lung cancer, by using the improved CARs and immune cells expressing these CARs as described herein. The invention also provides compositions comprising the engineered immune cells and methods of using these compositions. For example, provided herein are methods for treating a condition such as cancer in a subject using these compositions.

### General Techniques

The practice of the invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

The term “recombinant antigen receptor” as used herein refers broadly to a non-naturally occurring surface receptor that comprises an extracellular antigen-binding domain or an extracellular ligand-binding domain, a transmembrane domain and an intracellular domain. In some embodiments, the recombinant antigen receptor is a chimeric antigen receptor (CAR). In some embodiments, the intracellular domain of a recombinant antigen receptor comprises a co-stimulatory domain and an ITAM-containing domain. In some embodiments, the intracellular domain of a recombinant antigen receptor comprises an intracellular protein or a functional variant thereof (e.g., truncation(s), insertion(s), deletion(s) or substitution(s)).

The term “extracellular ligand-binding domain” or “extracellular antigen-binding domain” as used herein refers to a polypeptide that is capable of binding a ligand or an antigen or capable of interacting with a cell surface molecule, such as a ligand or a surface antigen. For example, the extracellular ligand-binding or antigen-binding domain may be chosen to recognize a ligand that acts as a cell surface marker on target cells associated with a particular disease state, e.g., a tumor-specific antigen. In some embodiments, the antigen-binding domain comprises an antibody, or an antigen binding fragment or an antigen binding portion of an antibody. In some embodiments, the antigen binding domain comprises an Fv or scFv, an Fab or scFab, an F(ab′)2 or a scF(ab′)2, an Fd, a monobody, a affibody, a camelid antibody, a VHH antibody, a single domain antibody, or a darpin. In some embodiments, the ligand-binding domain comprises a partner of a binding pair, such as a ligand that binds to a surface receptor, or an ectodomain of a surface receptor that binds to a ligand.

The term “stalk domain” or “hinge domain” are used interchangeably herein to refer to any polypeptide that functions to link the transmembrane domain to the extracellular ligand-binding domain. In particular, stalk domains are used to provide more flexibility and accessibility for the extracellular ligand-binding domain.

The term “intracellular signaling domain” refers to the portion of a protein which transduces the effector signal function signal and directs the cell to perform a specialized function.

A “co-stimulatory molecule” as used herein refers to the cognate binding partner on immune cells, e.g. T cells, that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

A “co-stimulatory ligand” refers to a molecule on an antigen presenting cell that specifically binds a cognate co-stimulatory signal molecule on a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory igand (ICOS-L), intercellular adhesion molecule (ICAM), CD30L, CD40, CD70, CD83, HLA-G, MICA, M1CB, HVEM, lymphotoxin β receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LTGHT, NKG2C, B7-H3, a ligand that specifically binds with CD83.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, IgE, IgD, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., DLL3). Antigen binding functions of an antibody can be performed by fragments of an intact antibody. Examples of binding fragments encompassed within the term “antigen binding fragment” of an antibody include Fab; Fab′; F(ab′)₂; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antigen binding fragment, an antibody conjugate, or a polypeptide that “specifically binds” to a target (e.g., DLL3 protein) is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. For example, an antibody that specifically binds to a DLL3 epitope is an antibody that binds this epitope with greater affinity, avidity, more readily, and/or with greater duration than it binds to other DLL3 epitopes or non-DLL3 epitopes. It is also understood that, for example, an antibody (or moiety or epitope) that specifically binds to a first target may or may not specifically bind to a second target. As such, “specific binding” does not necessarily require (although it can include) exclusive binding. Generally, but not necessarily, reference to binding means specific binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda MD)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by the hybridoma method first described by Kohler and Milstein, Nature 256:495, 1975, or may be made by recombinant DNA methods such as described in U.S. Pat. No. 4,816,567. The monoclonal antibodies may also be isolated from phage libraries generated using the techniques described in McCafferty et al., Nature 348:552-554, 1990, for example.

As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen binding subsequences of antibodies) that contain minimal sequence derived from non-human immunoglobulin. In one aspect, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “monovalent antibody” comprises one antigen binding site per molecule (e.g., IgG or Fab). In some instances, a monovalent antibody can have more than one antigen binding sites, but the binding sites are from different antigens.

A “bivalent antibody” comprises two antigen binding sites per molecule (e.g., IgG). In some instances, the two binding sites have the same antigen specificities. However, bivalent antibodies may be bispecific.

Recombinant antigen receptors e.g. chimeric antigen receptors (CARs) of the invention can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50, 1999 and Fellouse, F. A., et al, J. MoI. Biol., 373(4):924-40, 2007).

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As known in the art, a “constant region” of an antibody refers to the constant region of the antibody light chain or the constant region of the antibody heavy chain, either alone or in combination.

As used herein, “substantially pure” refers to material which is at least 50%, at least 60%, at least 70%, at least 80%, at least 90%, at least 95%, at least 97%, at least 98%, or even at least 99% pure (i.e., free from contaminants).

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant regions, CH2 and CH3.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, Ann. Rev. Immunol., 9:457-92, 1991; Capel et al., Immunomethods, 4:25-34, 1994; and de Haas et al., J. Lab. Clin. Med., 126:330-41, 1995. “FcR” also includes the neonatal receptor, FcRn, which is responsible for the transfer of maternal IgGs to the fetus (Guyer et al., J. Immunol., 117:587, 1976; and Kim et al., J. Immunol., 24:249, 1994).

The term “compete”, as used herein with regard to an antibody, means that a first antibody, or an antigen binding fragment (or portion) thereof, binds to an epitope in a manner sufficiently similar to the binding of a second antibody, or an antigen binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein “autologous” means that cells, a cell line, or population of cells used for treating patients are originating from said patient.

As used herein “allogeneic” means that cells or population of cells used for treating patients are not originating from said patient but from a donor.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of a tumor, remission of a disease (e.g., cancer), decreasing symptoms resulting from a disease e.g. (e.g., cancer), increasing the quality of life of those suffering from a disease (e.g., cancer), decreasing the dose of other medications required to treat a disease (e.g., cancer), delaying the progression of a disease (e.g., cancer), curing a disease (e.g., cancer), and/or prolonging survival of patients having a disease (e.g., cancer).

“Ameliorating” means a lessening or improvement of one or more symptoms as compared to not administering a CAR or engineered cell of the invention. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual”, “patient” or a “subject” are used interchangeably herein and is a mammal. Mammals include, but are not limited to, humans, monkeys, pigs, other farm animals, sport animals, pets, primates, horses, dogs, cats, rodents including mice, rats, guinea pigs, etc. A subject is a mammal and these terms are used interchangeably herein. In some embodiments, the subject is a human. In some embodiments, the subject is a non-human primate. In some embodiments, the subject is a human or a monkey, e.g. a cynomolgus monkey.

As used herein, “vector” means a construct, which is capable of delivering, and, in some embodiments, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutically acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Exemplary diluents for aerosol or parenteral administration include phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, PA, 1990; and Remington, The Science and Practice of Pharmacy 21st Ed. Mack Publishing, 2005).

The term “kₒₙ”, as used herein, refers to the rate constant for association of an antibody or scFv of a CAR to an antigen.

The term “k_{off}”, as used herein, refers to the rate constant for dissociation of an antibody or scFv of a CAR from the antibody/antigen complex.

The term “K_{D}”, as used herein, refers to the equilibrium dissociation constant of an antibody-antigen interaction or an scFv-antigen interaction.

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the invention encompasses not only the entire group listed as a whole, but each member of the group individually and all possible subgroups of the main group, but also the main group absent one or more of the group members. The invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the invention. The materials, methods, and examples are illustrative only and not intended to be limiting.

### Recombinant Antigen Receptors

The present invention provides recombinant antigen receptors comprising an extracellular antigen binding domain, a transmembrane domain, and an intracellular domain that comprises a co-stimulatory domain and an ITAM-containing domain. The intracellular signaling domain of a recombinant antigen receptor e.g. CAR according to the invention is responsible for intracellular signaling following the binding of an extracellular ligand-binding domain to the target resulting in the activation of the immune cell and immune response. The intracellular signaling domain has the ability to activate at least one of the normal effector functions of the immune cell in which the recombinant antigen receptor e.g. CAR is expressed. For example, the effector function of a T cell can be a cytolytic activity or helper activity including the secretion of cytokines.

In some embodiments, an intracellular signaling domain for use in a recombinant antigen receptor e.g. CAR of the invention can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Non-limiting examples of ITAM domains used in the invention can include as non-limiting examples those derived from TCRζ, FcRγ, FcRβ, FcRε, CD3γ, CD3δ, CD3ε, CD5, CD22, CD79a, CD79b and CD66d. In some embodiments the intracellular signaling domain of the CAR of the invention comprises a domain of a co-stimulatory molecule.

In some embodiments, the intracellular signaling domain of a CAR of the invention comprises a part of a co-stimulatory molecule selected from the group consisting of a fragment of 41BB (GenBank: AAA53133.) and CD28 (NP_006130.1).

In one aspect, the present invention provides recombinant antigen receptors comprising an extracellular antigen binding domain, a transmembrane domain, and an intracellular domain that comprises a co-stimulatory domain and an ITAM-containing domain, wherein:
(1) the ITAM-containing domain comprises one or more ITAM domains, e.g. three or more ITAM domains selected from the group consisting of a wildtype CD3g ITAM, CD3d ITAM, CD3e ITAM, CD3z1 ITAM, CD3z2 ITAM, CD3z3 ITAM, FcgRI ITAM, FcgRIIA ITAM, FcgRIIC ITAM, FcgRIIIA ITAM, FceRIG ITAM, and FceRIBITAM, and a variant of any one of the above, and wherein if the ITAM-containing domain comprises only three wildtype ITAM domains, the three ITAMs domains are not CD3z1, CD3z2 and CD3z3; and/or
(2) the intracellular domain further comprises one or more Lck recruiting motifs (LRM).

As used herein, CD3g refers to CD3 gamma (CD3γ), CD3d refers to CD3 delta (CD3δ), CD3e refers to CD3 epsilon (CD3ε), CD3z1 refers to CD3 zeta 1 (CD3ζ1), CD3z2 refers to CD3 zeta 2 (CD3ζ2), CD3z3 refers to CD3 zeta 3 (CD3ζ3).

As recited herein, the ITAM-containing domain can comprise a variety of configurations of the recited ITAMs, but does not include the configuration that is present in the wildtype CD3zeta ITAM-containing domain, which is only three ITAM domains, being CD3z1, CD3z2, CD3z3, in the order listed (amino to carboxy). In various embodiments, the ITAM-containing domain may have one or more ITAM domains, three or more ITAM domains (i.e., 3, 4, 5, 6, 7, 8, 9, 10 or more), and ITAM domains in multiples of three (e.g. an ITAM-containing domain comprising the six ITAMS CD3z1, CD3z2, CD3z3, CD3z1, CD3z2, CD3z3; in an embodiment, this ITAM-containing domain comprises the amino acid sequence of SEQ ID NO: 16). In various embodiments, the ITAM domains comprise the respective amino acid sequence set forth in Table 1: wildtype CD3g ITAM (SEQ ID NO: 25), CD3d ITAM (SEQ ID NO: 23), CD3e ITAM (SEQ ID NO: 24), CD3z1 ITAM (SEQ ID NO: 20), CD3z2 ITAM (SEQ ID NO: 21), CD3z3 ITAM (SEQ ID NO: 22).

In various embodiments, the ITAM-containing domain comprises the amino acid sequence of the wildtype CD3 zeta ITAM-containing domain (e.g. SEQ ID NO: 15 (also termed “CD3ζ cytoplasmic domain”)) except that at least one ITAM domain is replaced with a different ITAM domain. For example, in an embodiment of the invention, the ITAM-containing domain comprises the amino acid sequence of SEQ ID NO: 26, which is referred to as “CD3ζ (dzz),” indicating that it comprises the amino acid sequence of the wildtype CD3 zeta ITAM-containing domain except that it comprises the ITAM configuration CD3d ITAM, CD3z2 ITAM, CD3z3 ITAM instead of the ITAM configuration CD3z1 ITAM, CD3z2 ITAM, CD3z3 ITAM. In such an embodiment, the intervening amino acid sequences between the ITAM domains are the same as or comprise the corresponding amino acid sequences of the wildtype CD3 zeta ITAM-containing domain (e.g. the amino acid sequences of SEQ ID NO: 61 (intervening sequence between the first and second ITAM domains) and SEQ ID NO: 62 (intervening sequence between the second and third ITAM domains)). Similarly, in such an embodiment, the N-terminal amino acid sequence and C-terminal amino acid sequence of the ITAM-containing domain are the same as or comprise the corresponding N- and C-terminal amino acid sequence of the wildtype CD3 zeta ITAM-containing domain (e.g. the amino acid sequences of SEQ ID NO: 60 (N-terminal amino acid sequence) and SEQ ID NO: 63 (C-terminal amino acid sequence).

In the above example, the CD3d (or “delta”) ITAM domain is the replacement ITAM domain. In embodiments of the invention, the replacement ITAM domain can be any of those listed above (CD3g ITAM, CD3d ITAM, CD3e ITAM, CD3z1 ITAM, CD3z2 ITAM, CD3z3 ITAM, or a variant of any of these). The replacement ITAM domain can also be that of a protein other than the CD3 holocomplex, such as FcγRI, FcγRIIA, FcγRIIC, FcγRIIIA.

In various embodiments, the wildtype CD3 zeta ITAM-containing domain provides a framework that comprises three ITAM “slots,” a first ITAM slot, a second ITAM slot, and a third ITAM slot. For example, in the wildtype CD3 zeta ITAM-containing domain, the first ITAM slot contains CD3zeta ITAM zeta1 (e.g. comprising the amino acid sequence of SEQ ID NO: 20), the second ITAM slot contains CD3zeta ITAM zeta2 (e.g. comprising the amino acid sequence SEQ ID NO: 21), and the third ITAM slot contains CD3zeta ITAM zeta 3 (e.g. comprising the amino acid sequence SEQ ID NO: 22). Various embodiments of the recombinant antigen receptors of the invention comprise a variant of the wildtype CD3 zeta ITAM-containing domain, the variant differing from the wildtype in that the variant contains a different ITAM domain in at least one of the three slots. For example, in the example given above, the CD3 delta ITAM replaces the CD3zeta1 ITAM in the first ITAM slot, and the rest of the ITAM-containing domain retains the amino acid sequence of the wildtype CD3 zeta ITAM-containing domain.

In embodiments of the invention, any ITAM-containing domain can be inserted into any one or more slot to produce an ITAM-containing domain of the recombinant antigen receptors of the invention. For example, the ITAM-containing domain referred to herein as “CD3zeta (deg)” (e.g. comprising the amino acid sequence of SEQ ID NO: 29) has the CD3 delta ITAM delta domain in the first slot (e.g. comprising the amino acid sequence of SEQ ID NO: 23), the CD3 epsilon ITAM epsilon domain in the second slot (e.g. comprising the amino acid sequence of SEQ ID NO: 24), and the CD3 gamma ITAM gamma domain in the third slot (e.g. comprising the amino acid sequence of SEQ ID NO: 25).

In embodiments of the invention, the ITAM-containing domain of the recombinant antigen receptor comprises amino acid sequences in addition to the ITAM domain sequences. In an embodiment, the CD3 zeta cytoplasmic domain is the framework that provides the initial or N-terminal amino acid sequence amino to the first ITAM (e.g. comprising the amino acid sequence of SEQ ID NO: 60), C-terminal amino acid sequence carboxy to the last ITAM (e.g. comprising the amino acid sequence of SEQ ID NO: 63), sequence between the first and second ITAM domains, if there is a second ITAM domain (e.g. comprising the amino acid sequence of SEQ ID NO: 61), and sequence between the second and third ITAM domains, if there is a third ITAM domain (e.g. comprising the amino acid sequence of SEQ ID NO: 62). This pattern is applied to each set of three ITAMs. An example of this pattern is the amino acid sequence comprising SEQ ID NO: 16. As exemplified in SEQ ID NO: 16, one or more amino acids can be interposed between groups of ITAMs. For example, in SEQ ID NO: 16. a glycine is between the first group of three ITAMs and the second group of three ITAMs. In embodiments of the invention, a group of ITAMs has a sequence comprising the amino acid sequence of SEQ ID NO: 60 at the amino terminus of the group of ITAMs and has a sequence comprising the amino acid sequence of SEQ ID NO: 63 at the carboxy terminus of the group of ITAMs. In preferred embodiments, a group of ITAMs has three ITAMs with intervening sequences as set forth above.

In an embodiment in which the ITAM-containing domain comprises only one ITAM domain, the amino acid sequence of the ITAM-containing domain may comprise the amino acid sequence of SEQ ID NO: 60 amino to the ITAM amino acid sequence and the amino acid sequence of SEQ ID NO: 63 carboxy to the ITAM amino acid sequence. In embodiments that comprise a multiple of three ITAMs plus one ITAM (e.g. 4 ITAMs, 7 ITAMs), the same rule may apply to the ITAM outside the groups of three ITAMs (e.g. [3-ITAM group as described herein][SEQ ID NO: 60-single ITAM domain-SEQ ID NO: 63]).

In an embodiment in which the ITAM-containing domain comprises only two ITAM domains, the amino acid sequence may comprise the amino acid sequence of SEQ ID NO: 60 amino to the first ITAM amino acid sequence, the amino acid sequence of SEQ ID NO: 61 between the first and second ITAMs, and the amino acid sequence of SEQ ID NO: 63 carboxy to the second ITAM amino acid sequence. In embodiments that comprise a multiple of three ITAMs plus two ITAMs (e.g. 5 ITAMs, 8 ITAMs), the same rule may apply to the 2 ITAMs outside the groups of three ITAMs (e.g. [3-ITAM group as described herein][SEQ ID NO: 60-first ITAM domain-SEQ ID NO: 61-second ITAM domain-SEQ ID NO: 63]).

In an embodiment of the recombinant antigen receptors of the invention, the recombinant antigen receptor is a chimeric antigen receptor (CAR). In an embodiment, the CAR comprises an scFv. In an embodiment, the scFv has an amino acid sequence that comprises the amino acid sequence of SEQ ID NO: 43, 46 or 49. In certain embodiments, the scFv has binding affinity for DLL3. In certain embodiments, the scFv comprises an amino acid sequence which comprises at least 70%, for example at least 80%, or at least 90%, 95%, 97%, or 99% sequence identity with the amino acid sequence of SEQ ID NO: 43, 46 or 49.

In an embodiment of the recombinant antigen receptors of the invention, the antigen binding domain comprises a heavy chain variable domain (VH) and a light chain variable domain (VL). In some embodiments, the VH comprises the amino acid sequence of SEQ ID NO: 44 and the VL comprises the amino acid sequence of SEQ ID NO: 45, or the VH comprises the amino acid sequence of SEQ ID NO: 47 and the VL comprises the amino acid sequence of SEQ ID NO: 48, or the VH comprises the amino acid sequence of SEQ ID NO: 50 and the VL comprises the amino acid sequence of SEQ ID NO: 51. In some embodiments, a VH-VL linker amino acid sequence joins the VH and VL domains. In some embodiments, the VH-VL linker comprises between 1 and 5 copies of the amino acid sequence of SEQ ID NO: 52, e.g. 4 copies of SEQ ID NO: 52, as in SEQ ID NOs: 43, 46 and 49. In some embodiments, the VH is amino to the VL, and in some embodiments the VL is amino to the VH. In some embodiments, the antigen binding domain has binding affinity for DLL3. See WO2020/180591, which is incorporated herein by reference.

In an embodiment of the recombinant antigen receptors of the invention, the antigen binding domain is connected to the transmembrane domain by a hinge domain. In some embodiments, the hinge domain comprises the CD8 alpha hinge, for example the hinge domain comprises the amino acid sequence of SEQ ID NO: 11. In an embodiment, a linker sequence links the transmembrane domain to the intracellular domain. In an embodiment, the linker sequence comprises the amino acid sequence of SEQ ID NO: 13.

In an embodiment of the recombinant antigen receptors of the invention, the recombinant antigen receptor initially comprises a signal sequence, e.g the CD8 alpha signal sequence, e.g. the signal sequence comprises the amino acid sequence of SEQ ID NO: 10.

In an embodiment of the recombinant antigen receptor of the invention, the transmembrane domain comprises a CD8 alpha transmembrane domain, e.g. the transmembrane domain comprises the amino acid sequence of SEQ ID NO: 12.

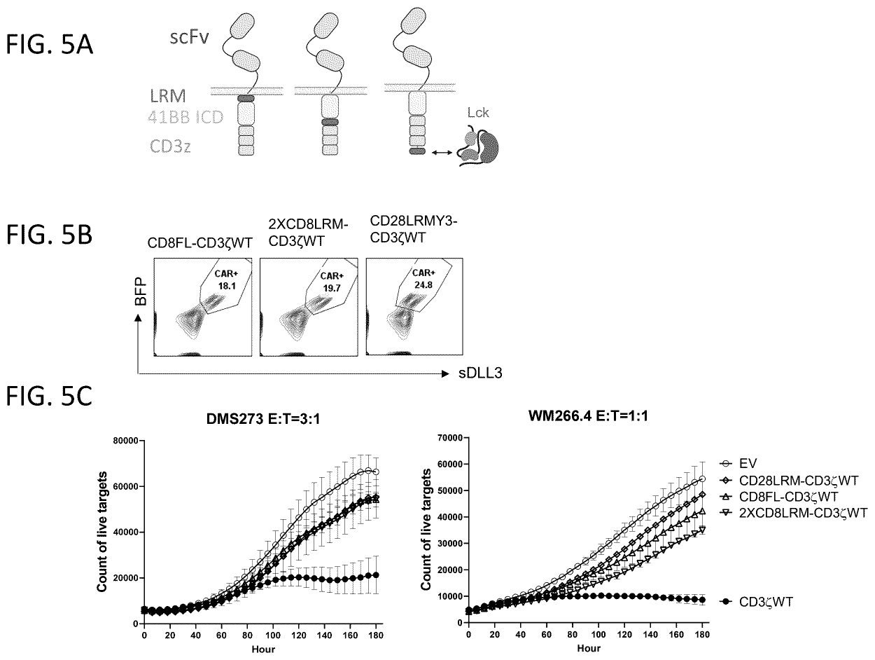

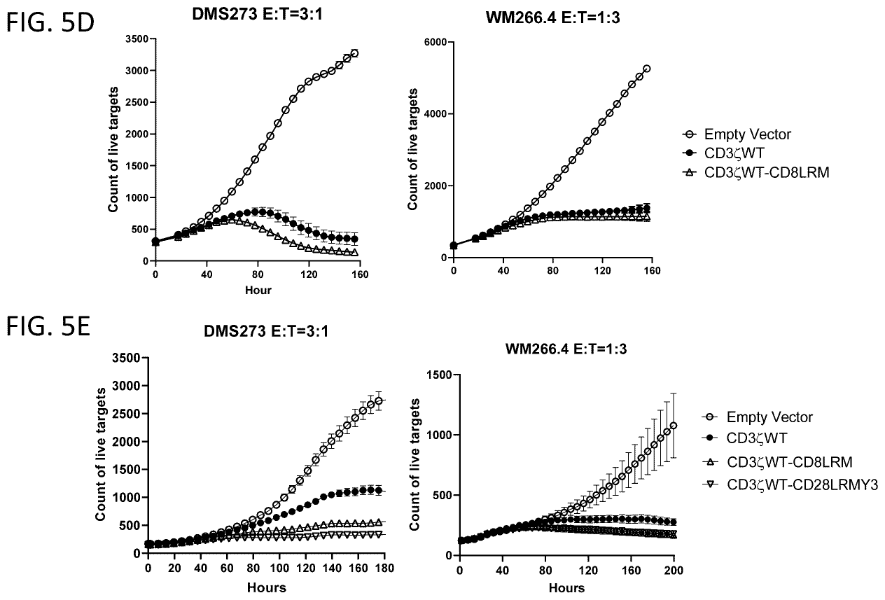

In an embodiment of the recombinant antigen receptor of the invention, the recombinant antigen receptor comprises an Lck recruiting motif (LRM). In an embodiment, a linker sequence connects the LRM positioned N-terminal to the intracellular domain of the recombinant antigen receptor as shown in FIG. 5A. In an embodiment, the amino terminal linker comprises from 1 to 4 copies e.g. 1 copy of the amino acid sequence of SEQ ID NO: 53 (termed “Linker 3”). In an embodiment, a linker sequence connects LRM positioned C-terminal to the intracellular domain of the recombinant antigen receptor. In an embodiment, the carboxy terminal linker comprises from 1 to 4 copies, e.g. 1 copy, of the amino acid sequence of SEQ ID NO: 52 (termed “Linker 2”) as shown in FIG. 5A.

In an embodiment of the recombinant antigen receptor of the invention, the intracellular domain comprises an LRM of CD4, CD8 or CD28. In an embodiment, the LRM comprises a CD4LRM (e.g. comprising the amino acid sequence of SEQ ID NO: 64), CD8 LRM-1 (e.g. comprising the amino acid sequence of SEQ ID NO: 55), 2×CD8LRM-1 (e.g. comprising the amino acid sequence of SEQ ID NO: 56), CD8LRM-2 (e.g. comprising the amino acid sequence of SEQ ID NO: 57), CD28LRM (e.g. comprising the amino acid sequence of SEQ ID NO: 58) or a CD28LRMY3 (e.g. comprising the amino acid sequence of SEQ ID NO: 59).

In an embodiment of the recombinant antigen receptors of the invention, the variant of a wildtype ITAM comprises an Ala substitution at the second position in the canonical YXX(L/I) ITAM motif (i.e., the Y+1 position) of a wildtype CD3z1, CD3z2, CD3z3, CD3d, CD3e, or CD3g. In some embodiments, the variant ITAM domain comprises the Ala substitution at the Y+1 position in the N-terminal YXX(L/I) motif. In some embodiments, the variant ITAM domain comprises the Ala substitution at the Y+1 position in the C-terminal YXX(L/I) motif. The variant ITAM domain can comprise the sequence of, for example, any one of SEQ ID NO: 65 (termed “CD3zeta ITAM zeta1 YA”), SEQ ID NO: 66 (termed “CD3zeta ITAM zeta2 YA”), SEQ ID NO: 67 (termed “CD3zeta ITAM zeta3 YA”), SEQ ID NO: 68 (termed “CD3delta ITAM delta YA”), SEQ ID NO: 69 (termed “CD3epsilon ITAM epsilon YA”), and SEQ ID NO: 70 (termed “CD3gamma ITAM gamma YA”).

An example of an ITAM-containing domain that comprises this variant of each of CD3z1, CD3z2, and CD3z3 comprises the amino acid sequence of SEQ ID NO: 35 (termed “CD3zeta YAYAYA”). An example of an ITAM-containing domain that comprises this variant of each of CD3z1, CD3z2, CD3z3, CD3d, CD3e, and CD3g comprises the amino acid sequence of SEQ ID NO: 37 (termed “CD3zeta zeta (zdzezg-6×YA)”).

In an embodiment of the recombinant antigen receptors of the invention, the co-stimulatory domain comprises a 4-1BB co-stimulatory domain. In an embodiment, the 4-1BB co-stimulatory domain comprises the amino acid sequence of SEQ ID NO: 14.

In an embodiment of the recombinant antigen receptors of the invention, the ITAM-containing domain consists of three ITAM domains or six ITAM domains, or generally a multiplicity of three ITAM domains, such as three, six or nine ITAM domains. One or more amino acids, for example, a single glycine, can join or link neighboring groups of three ITAM domains.

In an embodiment of the recombinant antigen receptors of the invention, the ITAM-containing domain comprises from N-terminus to C-terminus:
(a) CD3d ITAM, CD3z2 ITAM, CD3z3 ITAM (e.g. the ITAM-containing domain comprises the amino sequence of SEQ ID NO: 26);
(b) CD3e ITAM, CD3z2 ITAM, CD3z3 ITAM (e.g. the ITAM-containing domain comprises the amino sequence of SEQ ID NO: 27);
(c) CD3g ITAM, CD3z2 ITAM, CD3z3 ITAM (e.g. the ITAM-containing domain comprises the amino sequence of SEQ ID NO: 28);
(d) CD3d ITAM, CD3e ITAM, CD3g ITAM (e.g. the ITAM-containing domain comprises the amino sequence of SEQ ID NO: 29);
(e) CD3z1 ITAM, CD3z2 ITAM, CD3z3 ITAM, CD3z1 ITAM, CD3z2 ITAM, CD3z3 ITAM (e.g. the ITAM-containing domain comprises the amino sequence of SEQ ID NO: 16);
(f) CD3z1 ITAM, CD3d ITAM, CD3z2 ITAM, CD3e ITAM, CD3z3 ITAM, CD3g ITAM (e.g. the ITAM-containing domain comprises the amino sequence of SEQ ID NO: 30);
(g) CD3d ITAM, CD3z1 ITAM, CD3e ITAM, CD3z2 ITAM, CD3g ITAM, CD3z3 ITAM (e.g. the ITAM-containing domain comprises the amino sequence of SEQ ID NO: 31);
(h) CD3z1 ITAM, CD3z2 ITAM, CD3z3 ITAM, CD3d ITAM, CD3e ITAM, CD3g ITAM (e.g. the ITAM-containing domain comprises the amino sequence of SEQ ID NO: 32);
(i) CD3d ITAM, CD3e ITAM, CD3g ITAM, CD3z1 ITAM, CD3z2 ITAM, CD3z3 ITAM (e.g. the ITAM-containing domain comprises the amino sequence of SEQ ID NO: 33);
(j) CD3z1 (YAEL (SEQ ID NO: 152)) ITAM, CD3z2 ITAM, CD3z3 ITAM (e.g. the ITAM-containing domain comprises the amino sequence of SEQ ID NO: 34);
(k) CD3z1 (YAEL (SEQ ID NO: 152)) ITAM, CD3z2 (YAEL (SEQ ID NO: 152)) ITAM, CD3z3 (YAGL (SEQ ID NO: 153)) ITAM (e.g. the ITAM-containing domain comprises the amino sequence of SEQ ID NO: 35);
(l) CD3z1 (YAEL (SEQ ID NO: 152)) (e.g. the ITAM-containing domain comprises the amino sequence of SEQ ID NO: 36);
(m) CD3z1 (YAEL (SEQ ID NO: 152)) ITAM, CD3d (YAPL (SEQ ID NO: 154)) ITAM, CD3z2 (YAEL (SEQ ID NO: 152)) ITAM, CD3e (YAPI (SEQ ID NO: 155)) ITAM, CD3z3 (YAGL (SEQ ID NO: 153)) ITAM, CD3g (YAPL (SEQ ID NO: 154)) ITAM (e.g. the ITAM-containing domain comprises the amino sequence of SEQ ID NO: 37); or
(n) CD3d (YAPL (SEQ ID NO: 154)) ITAM, CD3z1 (YAEL (SEQ ID NO: 152)) ITAM, CD3e (YAPI (SEQ ID NO: 155)) ITAM, CD3z2 (YAEL (SEQ ID NO: 152)) ITAM, CD3g (YAPL (SEQ ID NO: 154)) ITAM, CD3z3 (YAGL (SEQ ID NO: 153)) ITAM (e.g. the ITAM-containing domain comprises the amino sequence of SEQ ID NO: 38).

TABLE 1

| | | SEQ ID |
|---|---|---|
| Plasmid Feature | Amino acid sequence | NO: |
| ZAP70 | MPDPAAHLPFFYGSISRAEAEEHLKLAGMADGLFLLRQCLRSLGG | 1 |
| | YVLSLVHDVRFHHFPIERQLNGTYAIAGGKAHCGPAELCEFYSRDP | |
| | DGLPCNLRKPCNRPSGLEPQPGVFDCLRDAMVRDYVRQTWKLEG | |
| | EALEQAIISQAPQVEKLIATTAHERMPWYHSSLTREEAERKLYSGA | |
| | QTDGKFLLRPRKEQGTYALSLIYGKTVYHYLISQDKAGKYCIPEGT | |
| | KFDTLWQLVEYLKLKADGLIYCLKEACPNSSASNASGAAAPTLPA | |
| | HPSTLTHPQRRIDTLNSDGYTPEPARITSPDKPRPMPMDTSVYESPY | |
| | SDPEELKDKKLFLKRDNLLIADIELGCGNFGSVRQGVYRMRKKQI | |
| | DVAIKVLKQGTEKADTEEMMREAQIMHQLDNPYIVRLIGVCQAE | |
| | ALMLVMEMAGGGPLHKFLVGKREEIPVSNVAELLHQVSMGMKY | |
| | LEEKNFVHRDLAARNVLLVNRHYAKISDFGLSKALGADDSYYTA | |
| | RSAGKWPLKWYAPECINFRKFSSRSDVWSYGVTMWEALSYGQKP | |
| | YKKMKGPEVMAFIEQGKRMECPPECPPELYALMSDCWIYKWEDR | |
| | PDFLTVEQRMRACYYSLASKVEGPPGSTQKAEAACA | |
| Lck | MGCGCSSHPEDDWMENIDVCENCHYPIVPLDGKGTLLIRNGSEVR | 2 |
| | DPLVTYEGSNPPASPLQDNLVIALHSYEPSHDGDLGFEKGEQLRIL | |
| | EQSGEWWKAQSLTTGQEGFIPFNFVAKANSLEPEPWFFKNLSRKD | |
| | AERQLLAPGNTHGSFLIRESESTAGSFSLSVRDFDQNQGEVVKHYK | |
| | IRNLDNGGFYISPRITFPGLHELVRHYTNASDGLCTRLSRPCQTQKP | |
| | QKPWWEDEWEVPRETLKLVERLGAGQFGEVWMGYYNGHTKVA | |
| | VKSLKQGSMSPDAFLAEANLMKQLQHQRLVRLYAVVTQEPIYIIT | |
| | EYMENGSLVDFLKTPSGIKLTINKLLDMAAQIAEGMAFIEERNYIH | |
| | RDLRAANILVSDTLSCKIADFGLARLIEDNEYTAREGAKFPIKWTA | |
| | PEAINYGTFTIKSDVWSFGILLTEIVTHGRIPYPGMTNPEVIQNLERG | |
| | YRMVRPDNCPEELYQLMRLCWKERPEDRPTFDYLRSVLEDFFTAT | |
| | EGQYQPQP | |
| Truncated | MGCGCSSHPEDDWMENIDVCENCHYPIVPLDGKGTLLIRNGSEVR | 3 |
| Lck | DPLVTYEGSNPPASPLQDNLVIALHSYEPSHDGDLGFEKGEQLRIL | |
| | EQSGEWWKAQSLTTGQEGFIPFNFVAKANSLEPEPWFFKNLSRKD | |
| | AERQLLAPGNTHGSFLIRESESTAGSFSLSVRDFDQNQGEVVKHYK | |
| | IRNLDNGGFYISPRITFPGLHELVRHYTNASDGLCTRLSRPCQTQKP | |
| | QKPWWEDEWEVPRET | |
| Fyn | MGCVQCKDKEATKLTEERDGSLNQSSGYRYGTDPTPQHYPSFGV | 4 |
| | TSIPNYNNFHAAGGQGLTVFGGVNSSSHTGTLRTRGGTGVTLFVA | |
| | LYDYEARTEDDLSFHKGEKFQILNSSEGDWWEARSLTTGETGYIPS | |
| | NYVAPVDSIQAEEWYFGKLGRKDAERQLLSFGNPRGTFLIRESETT | |
| | KGAYSLSIRDWDDMKGDHVKHYKIRKLDNGGYYITTRAQFETLQ | |
| | QLVQHYSERAAGLCCRLVVPCHKGMPRLTDLSVKTKDVWEIPRE | |
| | SLQLIKRLGNGQFGEVWMGTWNGNTKVAIKTLKPGTMSPESFLEE | |
| | AQIMKKLKHDKLVQLYAVVSEEPIYIVTEYMNKGSLLDFLKDGEG | |
| | RALKLPNLVDMAAQVAAGMAYIERMNYIHRDLRSANILVGNGLI | |
| | CKIADFGLARLIEDNEYTARQGAKFPIKWTAPEAALYGRFTIKSDV | |
| | WSFGILLTELVTKGRVPYPGMNNREVLEQVERGYRMPCPQDCPIS | |
| | LHELMIHCWKKDPEERPTFEYLQSFLEDYFTATEPQYQPGENL | |
| Truncated | MGCVQCKDKEATKLTEERDGSLNQSSGYRYGTDPTPQHYPSFGV | 5 |
| Fyn | TSIPNYNNFHAAGGQGLTVFGGVNSSSHTGTLRTRGGTGVTLFVA | |
| | LYDYEARTEDDLSFHKGEKFQILNSSEGDWWEARSLTTGETGYIPS | |
| | NYVAPVDSIQAEEWYFGKLGRKDAERQLLSFGNPRGTFLIRESETT | |
| | KGAYSLSIRDWDDMKGDHVKHYKIRKLDNGGYYITTRAQFETLQ | |
| | QLVQHYSERAAGLCCRLVVPCHKGMPRLTDLSVKTKDVWEIPRE | |
| | S | |
| Syk | MASSGMADSANHLPFFFGNITREEAEDYLVQGGMSDGLYLLRQSR | 6 |
| | NYLGGFALSVAHGRKAHHYTIERELNGTYAIAGGRTHASPADLCH | |
| | YHSQESDGLVCLLKKPFNRPQGVQPKTGPFEDLKENLIREYVKQT | |
| | WNLQGQALEQAIISQKPQLEKLIATTAHEKMPWFHGKISREESEQI | |
| | VLIGSKTNGKFLIRARDNNGSYALCLLHEGKVLHYRIDKDKTGKL | |
| | SIPEGKKFDTLWQLVEHYSYKADGLLRVLTVPCQKIGTQGNVNFG | |
| | GRPQLPGSHPATWSAGGIISRIKSYSFPKPGHRKSSPAQGNRQESTV | |
| | SFNPYEPELAPWAADKGPQREALPMDTEVYESPYADPEEIRPKEV | |
| | YLDRKLLTLEDKELGSGNFGTVKKGYYQMKKVVKTVAVKILKNE | |
| | ANDPALKDELLAEANVMQQLDNPYIVRMIGICEAESWMLVMEMA | |
| | ELGPLNKYLQQNRHVKDKNIIELVHQVSMGMKYLEESNFVHRDL | |
| | AARNVLLVTQHYAKISDFGLSKALRADENYYKAQTHGKWPVKW | |
| | YAPECINYYKFSSKSDVWSFGVLMWEAFSYGQKPYRGMKGSEVT | |
| | AMLEKGERMGCPAGCPREMYDLMNLCWTYDVENRPGFAAVELR | |
| | LRNYYYDVVN | |
| Truncated | MASSGMADSANHLPFFFGNITREEAEDYLVQGGMSDGLYLLRQSR | 7 |
| Syk | NYLGGFALSVAHGRKAHHYTIERELNGTYAIAGGRTHASPADLCH | |
| | YHSQESDGLVCLLKKPFNRPQGVQPKTGPFEDLKENLIREYVKQT | |
| | WNLQGQALEQAIISQKPQLEKLIATTAHEKMPWFHGKISREESEQI | |
| | VLIGSKTNGKFLIRARDNNGSYALCLLHEGKVLHYRIDKDKTGKL | |
| | SIPEGKKFDTLWQLVEHYSYKADGLLRVLTVPCQKIGTQGNVNFG | |
| | GRPQLPGSHPATWSAGGIISRIKSYSFPKPGHRKSSPAQGNRQESTV | |
| | SFNPYEPELAPWAADKGPQREALPMDTEVYESPYADPEEIRPKEV | |
| | YLDRKLL | |
| LAT | MEEAILVPCVLGLLLLPILAMLMALCVHCHRLPGSYDSTSSDSLYP | 8 |
| | RGIQFKRPHTVAPWPPAYPPVTSYPPLSQPDLLPIPRSPQPLGGSHR | |
| | TPSSRRDSDGANSVASYENEGASGIRGAQAGWGVWGPSWTRLTP | |
| | VSLPPEPACEDADEDEDDYHNPGYLVVLPDSTPATSTAAPSAPALS | |
| | TPGIRDSAFSMESIDDYVNVPESGESAEASLDGSREYVNVSQELHP | |
| | GAAKTEPAALSSQEAEEVEEEGAPDYENLQELN | |
| Unc119 | MKVKKGGGGAGTATESAPGPSGQSVAPIPQPPAESESGSESEPDAG | 9 |
| | PGPRPGPLQRKQPIGPEDVLGLQRITGDYLCSPEENIYKIDFVRFKIR | |
| | DMDSGTVLFEIKKPPVSERLPINRRDLDPNAGRFVRYQFTPAFLRL | |
| | RQVGATVEFTVGDKPVNNFRMIERHYFRNQLLKSFDFHFGFCIPSS | |
| | KNTCEHIYDFPPLSEELISEMIRHPYETQSDSFYFVDDRLVMHNKA | |
| | DYSYSGTP | |
| CD8α [alpha] | MALPVTALLLPLALLLHAARP | 10 |
| signal | | |
| sequence | | |
| CD8α [alpha] | TTTPAPRPPTPAPTIASQPLSLRPEACRPAAGGAVHTRGLDFACD | 11 |
| hinge | | |
| CD8α [alpha] | IYIWAPLAGTCGVLLLSLVIT | 12 |
| trans- | | |
| membrane | | |
| CD8α [alpha] | LYC | 13 |
| cytoplasmic | | |
| sequence | | |
| (truncated) | | |
| 4-1BB | KRGRKKLLYIFKQPFMRPVQTTQEEDGCSCRFPEEEEGGCEL | 14 |
| (TNFRSF9, | | |
| CD137) | | |
| cytoplasmic | | |
| domain | | |
| CD3zeta | RVKFSRSADAPAYQQGQNQLYNELNLGRREEYDVLDKRRGRDPE | 15 |
| cytoplasmic | MGGKPRRKNPQEGLYNELQKDKMAEAYSEIGMKGERRRGKGHD | |
| domain | GLYQGLSTATKDTYDALHMQALPPR | |
| CD3zeta zeta | RVKFSRSADAPAYQQGQNQLYNELNLGRREEYDVLDKRRGRDPE | 16 |
| concatenated | MGGKPRRKNPQEGLYNELQKDKMAEAYSEIGMKGERRRGKGHD | |
| cytoplasmic | GLYQGLSTATKDTYDALHMQALPPRGRVKFSRSADAPAYQQGQN | |
| domain | QLYNELNLGRREEYDVLDKRRGRDPEMGGKPRRKNPQEGLYNEL | |
| | QKDKMAEAYSEIGMKGERRRGKGHDGLYQGLSTATKDTYDALH | |
| | MQALPPR | |
| CD3 delta | GHETGRLSGAADTQALLRNDQVYQPLRDRDDAQYSHLGGNWAR | 17 |
| cytoplasmic | NK | |
| domain | | |
| CD3 epsilon | KNRKAKAKPVTRGAGAGGRQRGQNKERPPPVPNPDYEPIRKGQR | 18 |
| cytoplasmic | DLYSGLNQRRI | |
| domain | | |
| CD3 gamma | GQDGVRQSRASDKQTLLPNDQLYQPLKDREDDQYSHLQGNQLRR | 19 |
| cytoplasmic | N | |
| domain | | |
| CD3 zeta | APAYQQGQNQLYNELNLGRREEYDVLDKR | 20 |
| ITAM zeta1 | | |
| CD3 zeta | PRRKNPQEGLYNELQKDKMAEAYSEIGM | 21 |
| ITAM zeta2 | | |
| CD3 zeta | ERRRGKGHDGLYQGLSTATKDTYDALHMQ | 22 |
| ITAM zeta3 | | |
| CD3 delta | DTQALLRNDQVYQPLRDRDDAQYSHLGGN | 23 |
| ITAM delta | | |
| CD3 epsilon | ERPPPVPNPDYEPIRKGQRDLYSGLNQR | 24 |
| ITAM epsilon | | |
| CD3 gamma | DKQTLLPNDQLYQPLKDREDDQYSHLQGN | 25 |
| ITAM gamma | | |
| CD3 zeta | RVKFSRSADDTQALLRNDQVYQPLRDRDDAQYSHLGGNRGRDPE | 26 |
| (dzz) | MGGKPRRKNPQEGLYNELQKDKMAEAYSEIGMKGERRRGKGHD | |
| | GLYQGLSTATKDTYDALHMQALPPR | |
| CD3 zeta | RVKFSRSADERPPPVPNPDYEPIRKGQRDLYSGLNQRRGRDPEMG | 27 |
| (ezz) | GKPRRKNPQEGLYNELQKDKMAEAYSEIGMKGERRRGKGHDGL | |
| | YQGLSTATKDTYDALHMQALPPR | |
| CD3 zeta | RVKFSRSADDKQTLLPNDQLYQPLKDREDDQYSHLQGNRGRDPE | 28 |
| (gzz) | MGGKPRRKNPQEGLYNELQKDKMAEAYSEIGMKGERRRGKGHD | |
| | GLYQGLSTATKDTYDALHMQALPPR | |
| CD3 zeta | RVKFSRSADDTQALLRNDQVYQPLRDRDDAQYSHLGGNRGRDPE | 29 |
| (deg) | MGGKERPPPVPNPDYEPIRKGQRDLYSGLNQRKGDKQTLLPNDQL | |
| | YQPLKDREDDQYSHLQGNALPPR | |
| CD3 zeta zeta | RVKFSRSADAPAYQQGQNQLYNELNLGRREEYDVLDKRRGRDPE | 30 |
| (zdzezg) | MGGKDTQALLRNDQVYQPLRDRDDAQYSHLGGNKGPRRKNPQE | |
| | GLYNELQKDKMAEAYSEIGMALPPRGRVKFSRSADERPPPVPNPD | |
| | YEPIRKGQRDLYSGLNQRRGRDPEMGGKERRRGKGHDGLYQGLS | |
| | TATKDTYDALHMQKGDKQTLLPNDQLYQPLKDREDDQYSHLQG | |
| | NALPPR | |
| CD3 zeta zeta | RVKFSRSADDTQALLRNDQVYQPLRDRDDAQYSHLGGNRGRDPE | 31 |
| (dzezgz) | MGGKAPAYQQGQNQLYNELNLGRREEYDVLDKRKGERPPPVPNP | |
| | DYEPIRKGQRDLYSGLNQRALPPRGRVKFSRSADPRRKNPQEGLY | |
| | NELQKDKMAEAYSEIGMRGRDPEMGGKDKQTLLPNDQLYQPLK | |
| | DREDDQYSHLQGNKGERRRGKGHDGLYQGLSTATKDTYDALHM | |
| | QALPPR | |
| CD3 zeta zeta | RVKFSRSADAPAYQQGQNQLYNELNLGRREEYDVLDKRRGRDPE | 32 |
| (zzzdeg) | MGGKPRRKNPQEGLYNELQKDKMAEAYSEIGMKGERRRGKGHD | |
| | GLYQGLSTATKDTYDALHMQALPPRGRVKFSRSADDTQALLRND | |
| | QVYQPLRDRDDAQYSHLGGNRGRDPEMGGKERPPPVPNPDYEPIR | |
| | KGQRDLYSGLNQRKGDKQTLLPNDQLYQPLKDREDDQYSHLQGN | |
| | ALPPR | |
| CD3 zeta zeta | RVKFSRSADDTQALLRNDQVYQPLRDRDDAQYSHLGGNRGRDPE | 33 |
| (degzzz) | MGGKERPPPVPNPDYEPIRKGQRDLYSGLNQRKGDKQTLLPNDQL | |
| | YQPLKDREDDQYSHLQGNALPPRGRVKFSRSADAPAYQQGQNQL | |
| | YNELNLGRREEYDVLDKRRGRDPEMGGKPRRKNPQEGLYNELQK | |
| | DKMAEAYSEIGMKGERRRGKGHDGLYQGLSTATKDTYDALHMQ | |
| | ALPPR | |
| CD3 zeta YA | RVKFSRSADAPAYQQGQNQLYAELNLGRREEYDVLDKRRGRDPE | 34 |
| | MGGKPRRKNPQEGLYNELQKDKMAEAYSEIGMKGERRRGKGHD | |
| | GLYQGLSTATKDTYDALHMQALPPR | |
| CD3 zeta | RVKFSRSADAPAYQQGQNQLYAELNLGRREEYDVLDKRRGRDPE | 35 |
| YAYAYA | MGGKPRRKNPQEGLYAELQKDKMAEAYSEIGMKGERRRGKGHD | |
| | GLYAGLSTATKDTYDALHMQALPPR | |
| CD3 zeta | RVKFSRSADAPAYQQGQNQLYAELNLGRREEYDVLDKRALPPR | 36 |
| YAtrunc | | |
| CD3 zeta zeta | RVKFSRSADAPAYQQGQNQLYAELNLGRREEYDVLDKRRGRDPE | 37 |
| (zdzezg- | MGGKDTQALLRNDQVYAPLRDRDDAQYSHLGGNKGPRRKNPQE | |
| 6xYA) | GLYAELQKDKMAEAYSEIGMALPPRGRVKFSRSADERPPPVPNPD | |
| | YAPIRKGQRDLYSGLNQRRGRDPEMGGKERRRGKGHDGLYAGLS | |
| | TATKDTYDALHMQKGDKQTLLPNDQLYAPLKDREDDQYSHLQG | |
| | NALPPR | |
| CD3 zeta zeta | RVKFSRSADDTQALLRNDQVYAPLRDRDDAQYSHLGGNRGRDPE | 38 |
| (dzezgz- | MGGKAPAYQQGQNQLYAELNLGRREEYDVLDKRKGERPPPVPNP | |
| 6xYA) | DYAPIRKGQRDLYSGLNQRALPPRGRVKFSRSADPRRKNPQEGLY | |
| | AELQKDKMAEAYSEIGMRGRDPEMGGKDKQTLLPNDQLYAPLK | |
| | DREDDQYSHLQGNKGERRRGKGHDGLYAGLSTATKDTYDALHM | |
| | QALPPR | |
| BFP | MSELIKENMHMKLYMEGTVDNHHFKCTSEGEGKPYEGTQTMRIK | 39 |
| | VVEGGPLPFAFDILATSFLYGSKTFINHTQGIPDFFKQSFPEGFTWE | |
| | RVTTYEDGGVLTATQDTSLQDGCLIYNVKIRGVNFTSNGPVMQKK | |
| | TLGWEAFTETLYPADGGLEGRNDMALKLVGGSHLIANIKTTYRSK | |
| | KPAKNLKMPGVYYVDYRLERIKEANNETYVEQHEVAVARYCDLP | |
| | SKLGHKLN | |
| Linker 1 | GGSGG | 40 |
| (between BFP | | |
| and Furin | | |
| cleavage site) | | |
| Furin | RAKR | 41 |
| cleavage site | | |
| P2A peptide | ATNFSLLKQAGDVEENPGP | 42 |
| 10G1-K scFv | EVQLLESGGGLVQPGGSLRLSCAASGFTFSSYAMNWVRQAPGKG | 43 |
| | LEWVSTISGSGGSTYYADSVKGRFTISRDNSKNTLYLQMNSLRAE | |
| | DTAVFYCAIDPEYYDILTGGDYWGQGTLVTVSSGGGGSGGGGSG | |
| | GGGSGGGGSDIQMTQSPSAMSASVGDRVTITCRASQGISNYLAWF | |
| | QQKPGKVPKRLIYAASSLQSGVPSRFSGSGSGTEFTLTISSLQPEDF | |
| | ATYFCLQHDSFPLTFGGGTKVEIK | |
| 10G1-K VH | EVQLLESGGGLVQPGGSLRLSCAASGFTFSSYAMNWVRQAPGKG | 44 |
| | LEWVSTISGSGGSTYYADSVKGRFTISRDNSKNTLYLQMNSLRAE | |
| | DTAVFYCAIDPEYYDILTGGDYWGQGTLVTVSS | |
| 10G1-K VL | DIQMTQSPSAMSASVGDRVTITCRASQGISNYLAWFQQKPGKVPK | 45 |
| | RLIYAASSLQSGVPSRFSGSGSGTEFTLTISSLQPEDFATYFCLQHDS | |
| | FPLTFGGGTKVEIK | |
| 2G1 scFv | QLQLQESGPGLVKPSETLSLTCTVSGGSISSSSYYWGWIRQPPGKG | 46 |
| | LEWIGSIYYSGNIYHNPSLKSRVSISVDTSKNQFSLRLSSVTAADTA | |
| | VYYCAREIIVGATHFDYWGQGTLVTVSSGGGGSGGGGSGGGGSG | |
| | GGGSAIQMTQSPSSLSASVGDRVTITCRASQGIRNDLGWYQQKPG | |
| | KAPELLIYAASSLQSGVPSRFSGSGSGTDFTLTISSLQPEDFATYYCL | |
| | QDYNYPLTFGPGTKVDIK | |
| 2G1 VH | QLQLQESGPGLVKPSETLSLTCTVSGGSISSSSYYWGWIRQPPGKG | 47 |
| | LEWIGSIYYSGNIYHNPSLKSRVSISVDTSKNQFSLRLSSVTAADTA | |
| | VYYCAREIIVGATHFDYWGQGTLVTVSS | |
| 2G1 VL | AIQMTQSPSSLSASVGDRVTITCRASQGIRNDLGWYQQKPGKAPEL | 48 |
| | LIYAASSLQSGVPSRFSGSGSGTDFTLTISSLQPEDFATYYCLQDYN | |
| | YPLTFGPGTKVDIK | |
| 4H8 scFv | QVQLQQSGPGLVKPSQTLSLTCAISGDSVSSNSATWNWIRQSPSRG | 49 |
| | LEWLGRTYYRSKWYDDYAVSVKSRITINPDTSKNHLSLHLNSVTP | |
| | EDTAVYYCAGGGLVGAPDGFDVWGQGTMVTVSSGGGGSGGGGS | |
| | GGGGSGGGGSQSVLTQPPSASGTPGQRVTISCSGSSSNIGSDPVNW | |
| | YQQLPGTAPKLLIYSNNQRPSGVPDRFSGSKSGTSASLAISGLQSED | |
| | EADYYCSAWDDSLNGYVFGTGTKVTVL | |
| 4H8 VH | QVQLQQSGPGLVKPSQTLSLTCAISGDSVSSNSATWNWIRQSPSRG | 50 |
| | LEWLGRTYYRSKWYDDYAVSVKSRITINPDTSKNHLSLHLNSVTP | |
| | EDTAVYYCAGGGLVGAPDGFDVWGQGTMVTVSS | |
| 4H8 VL | QSVLTQPPSASGTPGQRVTISCSGSSSNIGSDPVNWYQQLPGTAPK | 51 |
| | LLIYSNNQRPSGVPDRFSGSKSGTSASLAISGLQSEDEADYYCSAW | |
| | DDSLNGYVFGTGTKVTVL | |
| Linker 2 (e.g. | GGGGS | 52 |
| Between CAR | | |
| and C- | | |
| terminal LRM | | |
| or between | | |
| CAR and N- | | |
| terminal | | |
| LRM) | | |
| Linker 3 (e.g. | GGGS | 53 |
| Between CAR | | |
| and N- | | |
| terminal LRM | | |
| or between | | |
| CAR and C- | | |
| terminal | | |
| LRM) | | |
| CD8 | LYCNHRNRRRVCKCPRPVVKSGDKPSLSARYV | 54 |
| cytoplasmic | | |
| domain (full | | |
| length) | | |
| CD8LRM-1 | RRVCKCPR | 55 |
| 2XCD8LRM-1 | RRVCKCPRRRVCKCPR | 56 |
| CD8LRM-2 | RVCKCPRPV | 57 |
| CD28LRM | YQPYAPPRDFAAYRS | 58 |
| CD28LRMY3 | FQPFAPPRDFAAFRS | 59 |
| N-term Seq 1 | RVKFSRSAD | 60 |
| Intervening | RGRDPEMGGK | 61 |
| Seq 2 | | |
| Intervening | KG | 62 |
| Seq 3 | | |
| C-term Seq 4 | ALPPR | 63 |
| CD4LRM | RMSQIKRLLSEKKTCQCP | 64 |
| CD3 zeta | APAYQQGQNQLYAELNLGRREEYDVLDKR | 65 |
| ITAM zeta1 | | |
| YA | | |
| CD3 zeta | PRRKNPQEGLYAELQKDKMAEAYSEIGM | 66 |
| ITAM zeta2 | | |
| YA | | |
| CD3 zeta | ERRRGKGHDGLYAGLSTATKDTYDALHMQ | 67 |
| ITAM zeta3 | | |
| YA | | |
| CD3 delta | DTQALLRNDQVYAPLRDRDDAQYSHLGGN | 68 |
| ITAM delta | | |
| YA | | |
| CD3 epsilon | ERPPPVPNPDYAPIRKGQRDLYSGLNQR | 69 |
| ITAM epsilon | | |
| YA | | |
| CD3 gamma | DKQTLLPNDQLYAPLKDREDDQYSHLQGN | 70 |
| ITAM gamma | | |
| YA | | |
| 4H8-R2S | MALPVTALLLPLALLLHAARPGGGGSCPYSNPSLCGGGGSCPYSN | 91 |
| Underlined is | PSLCGGGGSQVQLQQSGPGLVKPSQTLSLTCAISGDSVSSNSATWN | |
| the CD8 | WIRQSPSRGLEWLGRTYYRSKWYDDYAVSVKSRITINPDTSKNHL | |
| signal | SLHLNSVTPEDTAVYYCAGGGLVGAPDGFDVWGQGTMVTVSSG | |
| sequence | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKPRRKNPQEGLYNELQKDKMAEAYSEIGMK | |
| | GERRRGKGHDGLYQGLSTATKDTYDALHMQALPPR | |
| 4H8-R2S- | MALPVTALLLPLALLLHAARPGGGGSCPYSNPSLCGGGGSCPYSN | 92 |
| CD8 Full | PSLCGGGGSQVQLQQSGPGLVKPSQTLSLTCAISGDSVSSNSATWN | |
| length (FL)- | WIRQSPSRGLEWLGRTYYRSKWYDDYAVSVKSRITINPDTSKNHL | |
| CD3ζWT | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCNHRNRRRVCKCPRPVVKSGDKPSLSARYVK | |
| | RGRKKLLYIFKQPFMRPVQTTQEEDGCSCRFPEEEEGGCELRVKFS | |
| | RSADAPAYQQGQNQLYNELNLGRREEYDVLDKRRGRDPEMGGK | |
| | PRRKNPQEGLYNELQKDKMAEAYSEIGMKGERRRGKGHDGLYQ | |
| | GLSTATKDTYDALHMQALPPR | |
| 4H8-R2S- | MALPVTALLLPLALLLHAARPGGGGSCPYSNPSLCGGGGSCPYSN | 93 |
| 2XCD8LRM- | PSLCGGGGSQVQLQQSGPGLVKPSQTLSLTCAISGDSVSSNSATWN | |
| CD30ζWT | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITRRVCKCPRRRVCKCPRKRGRKKLLYIFKQPFMRP | |
| | VQTTQEEDGCSCRFPEEEEGGCELRVKFSRSADAPAYQQGQNQLY | |
| | NELNLGRREEYDVLDKRRGRDPEMGGKPRRKNPQEGLYNELQKD | |
| | KMAEAYSEIGMKGERRRGKGHDGLYQGLSTATKDTYDALHMQA | |
| | LPPR | |
| 4H8-R2S- | MALPVTALLLPLALLLHAARPGGGGSCPYSNPSLCGGGGSCPYSN | 94 |
| CD28LRM- | PSLCGGGGSQVQLQQSGPGLVKPSQTLSLTCAISGDSVSSNSATWN | |
| CD3ζWT | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCGGGSYQPYAPPRDFAAYRSGGGSKRGRKKL | |
| | LYIFKQPFMRPVQTTQEEDGCSCRFPEEEEGGCELRVKFSRSADAP | |
| | AYQQGQNQLYNELNLGRREEYDVLDKRRGRDPEMGGKPRRKNP | |
| | QEGLYNELQKDKMAEAYSEIGMKGERRRGKGHDGLYQGLSTAT | |
| | KDTYDALHMQALPPR | |
| 4H8-R2S- | MALPVTALLLPLALLLHAARPGGGGSCPYSNPSLCGGGGSCPYSN | 95 |
| CD28LRMY3- | PSLCGGGGSQVQLQQSGPGLVKPSQTLSLTCAISGDSVSSNSATWN | |
| CD3ζWT | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCGGGSFQPFAPPRDFAAFRSGGGSKRGRKKLL | |
| | YIFKQPFMRPVQTTQEEDGCSCRFPEEEEGGCELRVKFSRSADAPA | |
| | YQQGQNQLYNELNLGRREEYDVLDKRRGRDPEMGGKPRRKNPQ | |
| | EGLYNELQKDKMAEAYSEIGMKGERRRGKGHDGLYQGLSTATK | |
| | DTYDALHMQALPPR | |
| 4H8-R2S- | MALPVTALLLPLALLLHAARPGGGGSCPYSNPSLCGGGGSCPYSN | 96 |
| CD3ζ- | PSLCGGGGSQVQLQQSGPGLVKPSQTLSLTCAISGDSVSSNSATWN | |
| CD8LRM | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKPRRKNPQEGLYNELQKDKMAEAYSEIGMK | |
| | GERRRGKGHDGLYQGLSTATKDTYDALHMQALPPRGGGGSRVC | |
| | KCPRPV | |
| 4H8-R2S- | MALPVTALLLPLALLLHAARPGGGGSCPYSNPSLCGGGGSCPYSN | 97 |
| CD3ζWT- | PSLCGGGGSQVQLQQSGPGLVKPSQTLSLTCAISGDSVSSNSATWN | |
| CD28LRMY3 | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKPRRKNPQEGLYNELQKDKMAEAYSEIGMK | |
| | GERRRGKGHDGLYQGLSTATKDTYDALHMQALPPRGGGSFQPFA | |
| | PPRDFAAFRS | |
| 4H8-R2S- | MALPVTALLLPLALLLHAARPGGGGSCPYSNPSLCGGGGSCPYSN | 98 |
| CD3ζWT- | PSLCGGGGSQVQLQQSGPGLVKPSQTLSLTCAISGDSVSSNSATWN | |
| C2D8LRM | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKPRRKNPQEGLYNELQKDKMAEAYSEIGMK | |
| | GERRRGKGHDGLYQGLSTATKDTYDALHMQALPPRGGGSYQPY | |
| | APPRDFAAYRS | |
| 4H8-R2S- | MALPVTALLLPLALLLHAARPGGGGSCPYSNPSLCGGGGSCPYSN | 99 |
| BB- | PSLCGGGGSQVQLQQSGPGLVKPSQTLSLTCAISGDSVSSNSATWN | |
| FullCD8Cyto- | WIRQSPSRGLEWLGRTYYRSKWYDDYAVSVKSRITINPDTSKNHL | |
| CD3ζWT | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITKRGRKKLLYIFKQPFMRPVQTTQEEDGCSCRFPE | |
| | EEEGGCELLYCNHRNRRRVCKCPRPVVKSGDKPSLSARYVRVKFS | |
| | RSADAPAYQQGQNQLYNELNLGRREEYDVLDKRRGRDPEMGGK | |
| | PRRKNPQEGLYNELQKDKMAEAYSEIGMKGERRRGKGHDGLYQ | |
| | GLSTATKDTYDALHMQALPPR | |
| 4H8-R2S- | MALPVTALLLPLALLLHAARPGGGGSCPYSNPSLCGGGGSCPYSN | 100 |
| BB - | PSLCGGGGSQVQLQQSGPGLVKPSQTLSLTCAISGDSVSSNSATWN | |
| 2XCD8LRM- | WIRQSPSRGLEWLGRTYYRSKWYDDYAVSVKSRITINPDTSKNHL | |
| CD3ζWT | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITKRGRKKLLYIFKQPFMRPVQTTQEEDGCSCRFPE | |
| | EEEGGCELRRVCKCPRRRVCKCPRRVKFSRSADAPAYQQGQNQL | |
| | YNELNLGRREEYDVLDKRRGRDPEMGGKPRRKNPQEGLYNELQK | |
| | DKMAEAYSEIGMKGERRRGKGHDGLYQGLSTATKDTYDALHMQ | |
| | ALPPR | |
| 4H8-R2S- | MALPVTALLLPLALLLHAARPGGGGSCPYSNPSLCGGGGSCPYSN | 101 |
| CD3ζζ(zdzezg) | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKDTQALLRNDQVYQPLRDRDDAQYSHLGGN | |
| | KGPRRKNPQEGLYNELQKDKMAEAYSEIGMALPPRGRVKFSRSA | |
| | DERPPPVPNPDYEPIRKGQRDLYSGLNQRRGRDPEMGGKERRRGK | |
| | GHDGLYQGLSTATKDTYDALHMQKGDKQTLLPNDQLYQPLKDR | |
| | EDDQYSHLQGNALPPR | |
| 4H8-R2S- | MALPVTALLLPLALLLHAARPGGGGSCPYSNPSLCGGGGSCPYSN | 102 |
| CD3ζζ | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKPRRKNPQEGLYNELQKDKMAEAYSEIGMK | |
| | GERRRGKGHDGLYQGLSTATKDTYDALHMQALPPRGRVKFSRSA | |
| | DAPAYQQGQNQLYNELNLGRREEYDVLDKRRGRDPEMGGKPRR | |
| | KNPQEGLYNELQKDKMAEAYSEIGMKGERRRGKGHDGLYQGLST | |
| | ATKDTYDALHMQALPPR | |
| 4H8-R2S- | MALPVTALLLPLALLLHAARPGGGGSCPYSNPSLCGGGGSCPYSN | 103 |
| CD3ζζ | PSLCGGGGSQVQLQQSGPGLVKPSQTLSLTCAISGDSVSSNSATWN | |
| (YAYAYA) | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYAELNLGRREEYDV | |
| | LDKRRGRDPEMGGKPRRKNPQEGLYAELQKDKMAEAYSEIGMK | |
| | GERRRGKGHDGLYAGLSTATKDTYDALHMQALPPRRVKFSRSAD | |
| | APAYQQGQNQLYAELNLGRREEYDVLDKRRGRDPEMGGKPRRK | |
| | NPQEGLYAELQKDKMAEAYSEIGMKGERRRGKGHDGLYAGLSTA | |
| | TKDTYDALHMQALPPR | |
| 4H8-R2S- | MALPVTALLLPLALLLHAARPGGGGSCPYSNPSLCGGGGSCPYSN | 104 |
| CD3ζζ | PSLCGGGGSQVQLQQSGPGLVKPSQTLSLTCAISGDSVSSNSATWN | |
| (zdzezg-6xYA) | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYAELNLGRREEYDV | |
| | LDKRRGRDPEMGGKDTQALLRNDQVYAPLRDRDDAQYSHLGGN | |
| | KGPRRKNPQEGLYAELQKDKMAEAYSEIGMALPPRGRVKFSRSA | |
| | DERPPPVPNPDYAPIRKGQRDLYSGLNQRRGRDPEMGGKERRRGK | |
| | GHDGLYAGLSTATKDTYDALHMQKGDKQTLLPNDQLYAPLKDR | |
| | EDDQYSHLQGNALPPR | |
| 4H8-R2S- | MALPVTALLLPLALLLHAARPGGGGSCPYSNPSLCGGGGSCPYSN | 105 |
| CD3ζζ | PSLCGGGGSQVQLQQSGPGLVKPSQTLSLTCAISGDSVSSNSATWN | |
| (dzezgz-6xYA) | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADDTQALLRNDQVYAPLRDRDDAQYSH | |
| | LGGNRGRDPEMGGKAPAYQQGQNQLYAELNLGRREEYDVLDKR | |
| | KGERPPPVPNPDYAPIRKGQRDLYSGLNQRALPPRGRVKFSRSADP | |
| | RRKNPQEGLYAELQKDKMAEAYSEIGMRGRDPEMGGKDKQTLLP | |
| | NDQLYAPLKDREDDQYSHLQGNKGERRRGKGHDGLYAGLSTAT | |
| | KDTYDALHMQALPPR | |
| 4H8-R2S- | MALPVTALLLPLALLLHAARPGGGGSCPYSNPSLCGGGGSCPYSN | 106 |
| CD3ζζ(dzezgz) | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADDTQALLRNDQVYQPLRDRDDAQYSH | |
| | LGGNRGRDPEMGGKAPAYQQGQNQLYNELNLGRREEYDVLDKR | |
| | KGERPPPVPNPDYEPIRKGQRDLYSGLNQRALPPRGRVKFSRSADP | |
| | RRKNPQEGLYNELQKDKMAEAYSEIGMRGRDPEMGGKDKQTLLP | |
| | NDQLYQPLKDREDDQYSHLQGNKGERRRGKGHDGLYQGLSTAT | |
| | KDTYDALHMQALPPR | |
| 4H8-R2S- | MALPVTALLLPLALLLHAARPGGGGSCPYSNPSLCGGGGSCPYSN | 107 |
| CD3ζζ(degzzz) | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADDTQALLRNDQVYQPLRDRDDAQYSH | |
| | LGGNRGRDPEMGGKERPPPVPNPDYEPIRKGQRDLYSGLNQRKGD | |
| | KQTLLPNDQLYQPLKDREDDQYSHLQGNALPPRGRVKFSRSADAP | |
| | AYQQGQNQLYNELNLGRREEYDVLDKRRGRDPEMGGKPRRKNP | |
| | QEGLYNELQKDKMAEAYSEIGMKGERRRGKGHDGLYQGLSTAT | |
| | KDTYDALHMQALPPR | |
| 4H8-R2S- | MALPVTALLLPLALLLHAARPGGGGSCPYSNPSLCGGGGSCPYSN | 108 |
| CD3ζζ(zzzdeg) | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKPRRKNPQEGLYNELQKDKMAEAYSEIGMK | |
| | GERRRGKGHDGLYQGLSTATKDTYDALHMQALPPRGRVKFSRSA | |
| | DDTQALLRNDQVYQPLRDRDDAQYSHLGGNRGRDPEMGGKERP | |
| | PPVPNPDYEPIRKGQRDLYSGLNQRKGDKQTLLPNDQLYQPLKDR | |
| | EDDQYSHLQGNALPPR | |
| 4H8-R2S- | MALPVTALLLPLALLLHAARPGGGGSCPYSNPSLCGGGGSCPYSN | 109 |
| CD3ζ | PSLCGGGGSQVQLQQSGPGLVKPSQTLSLTCAISGDSVSSNSATWN | |
| (YAYAYA) | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYAELNLGRREEYDV | |
| | LDKRRGRDPEMGGKPRRKNPQEGLYAELQKDKMAEAYSEIGMK | |
| | GERRRGKGHDGLYAGLSTATKDTYDALHMQALPPR | |
| 4H8-R2S- | MALPVTALLLPLALLLHAARPGGGGSCPYSNPSLCGGGGSCPYSN | 110 |
| CD3ζ(dzz) | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADDTQALLRNDQVYQPLRDRDDAQYSH | |
| | LGGNRGRDPEMGGKPRRKNPQEGLYNELQKDKMAEAYSEIGMK | |
| | GERRRGKGHDGLYQGLSTATKDTYDALHMQALPPR | |
| 4H8-R2S- | MALPVTALLLPLALLLHAARPGGGGSCPYSNPSLCGGGGSCPYSN | 111 |
| CD3ζ(ezz) | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADERPPPVPNPDYEPIRKGQRDLYSGLN | |
| | QRRGRDPEMGGKPRRKNPQEGLYNELQKDKMAEAYSEIGMKGE | |
| | RRRGKGHDGLYQGLSTATKDTYDALHMQALPPR | |
| 4H8-R2S- | MALPVTALLLPLALLLHAARPGGGGSCPYSNPSLCGGGGSCPYSN | 112 |
| CD3ζ(gzz) | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADDKQTLLPNDQLYQPLKDREDDQYSH | |
| | LQGNRGRDPEMGGKPRRKNPQEGLYNELQKDKMAEAYSEIGMK | |
| | GERRRGKGHDGLYQGLSTATKDTYDALHMQALPPR | |
| 4H8-R2S- | MALPVTALLLPLALLLHAARPGGGGSCPYSNPSLCGGGGSCPYSN | 113 |
| CD3ζ(dzg) | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADDTQALLRNDQVYQPLRDRDDAQYSH | |
| | LGGNRGRDPEMGGKERPPPVPNPDYEPIRKGQRDLYSGLNQRKGD | |
| | KQTLLPNDQLYQPLKDREDDQYSHLQGNALPPR | |
| 4H8-R2S- | MALPVTALLLPLALLLHAARPGGGGSCPYSNPSLCGGGGSCPYSN | 114 |
| CD3ζYA | PSLCGGGGSQVQLQQSGPGLVKPSQTLSLTCAISGDSVSSNSATWN | |
| | WIRQSPSRGLEWLGRTYYRSKWYDDYAVSVKSRITINPDTSKNHL | |
| | SLHLNSVTPEDTAVYYCAGGGLVGAPDGFDVWGQGTMVTVSSG | |
| | GGGSGGGGSGGGGSGGGGSQSVLTQPPSASGTPGQRVTISCSGSSS | |
| | NIGSDPVNWYQQLPGTAPKLLIYSNNQRPSGVPDRFSGSKSGTSAS | |
| | LAISGLQSEDEADYYCSAWDDSLNGYVFGTGTKVTVLTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYAELNLGRREEYDV | |
| | LDKRRGRDPEMGGKPRRKNPQEGLYNELQKDKMAEAYSEIGMK | |
| | GERRRGKGHDGLYQGLSTATKDTYDALHMQALPPR | |
| 2G1-RSR | MALPVTALLLPLALLLHAARPGGGGSCPYSNPSLCGGGGSQLQLQ | 115 |
| | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYNELNLGRREEYDVLDKRR | |
| | GRDPEMGGKPRRKNPQEGLYNELQKDKMAEAYSEIGMKGERRR | |
| | GKGHDGLYQGLSTATKDTYDALHMQALPPR | |
| 2G1-RSR- | MALPVTALLLPLALLLHAARPGGGGSCPYSNPSLCGGGGSQLQLQ | 116 |
| CD8 FL- | ESGPGLVKPSETLSLTCTVSGGSISSSSYYWGWIRQPPGKGLEWIGS | |
| CD3ζWT | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCNHRNRRRVCKCPRPVVKSGDKPSLSARYVKRGRKKL | |
| | LYIFKQPFMRPVQTTQEEDGCSCRFPEEEEGGCELRVKFSRSADAP | |
| | AYQQGQNQLYNELNLGRREEYDVLDKRRGRDPEMGGKPRRKNP | |
| | QEGLYNELQKDKMAEAYSEIGMKGERRRGKGHDGLYQGLSTAT | |
| | KDTYDALHMQALPPR | |
| 2G1-RSR- | MALPVTALLLPLALLLHAARPGGGGSCPYSNPSLCGGGGSQLQLQ | 117 |
| 2XCD8LRM- | ESGPGLVKPSETLSLTCTVSGGSISSSSYYWGWIRQPPGKGLEWIGS | |
| CD3ζWT | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITRRVCKCPRRRVCKCPRKRGRKKLLYIFKQPFMRPVQTTQE | |
| | EDGCSCRFPEEEEGGCELRVKFSRSADAPAYQQGQNQLYNELNLG | |
| | RREEYDVLDKRRGRDPEMGGKPRRKNPQEGLYNELQKDKMAEA | |
| | YSEIGMKGERRRGKGHDGLYQGLSTATKDTYDALHMQALPPR | |
| 2G1-RSR- | MALPVTALLLPLALLLHAARPGGGGSCPYSNPSLCGGGGSQLQLQ | 118 |
| CD28LRM- | ESGPGLVKPSETLSLTCTVSGGSISSSSYYWGWIRQPPGKGLEWIGS | |
| CD3ζWT | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCGGGSYQPYAPPRDFAAYRSGGGSKRGRKKLLYIFKQP | |
| | FMRPVQTTQEEDGCSCRFPEEEEGGCELRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 2G1-RSR- | MALPVTALLLPLALLLHAARPGGGGSCPYSNPSLCGGGGSQLQLQ | 119 |
| CD28LRMY3- | ESGPGLVKPSETLSLTCTVSGGSISSSSYYWGWIRQPPGKGLEWIGS | |
| CD3ζWT | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCGGGSFQPFAPPRDFAAFRSGGGSKRGRKKLLYIFKQPF | |
| | MRPVQTTQEEDGCSCRFPEEEEGGCELRVKFSRSADAPAYQQGQN | |
| | QLYNELNLGRREEYDVLDKRRGRDPEMGGKPRRKNPQEGLYNEL | |
| | QKDKMAEAYSEIGMKGERRRGKGHDGLYQGLSTATKDTYDALH | |
| | MQALPPR | |
| 2G1-RSR- | MALPVTALLLPLALLLHAARPGGGGSCPYSNPSLCGGGGSQLQLQ | 120 |
| CD3ζ- | ESGPGLVKPSETLSLTCTVSGGSISSSSYYWGWIRQPPGKGLEWIGS | |
| CD8LRM | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYNELNLGRREEYDVLDKRR | |
| | GRDPEMGGKPRRKNPQEGLYNELQKDKMAEAYSEIGMKGERRR | |
| | GKGHDGLYQGLSTATKDTYDALHMQALPPRGGGGSRVCKCPRPV | |
| 2G1-RSR- | MALPVTALLLPLALLLHAARPGGGGSCPYSNPSLCGGGGSQLQLQ | 121 |
| CD3ζWT | ESGPGLVKPSETLSLTCTVSGGSISSSSYYWGWIRQPPGKGLEWIGS | |
| CD28LRMY3- | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYNELNLGRREEYDVLDKRR | |
| | GRDPEMGGKPRRKNPQEGLYNELQKDKMAEAYSEIGMKGERRR | |
| | GKGHDGLYQGLSTATKDTYDALHMQALPPRGGGSFQPFAPPRDF | |
| | AAFRS | |
| 2G1-RSR- | MALPVTALLLPLALLLHAARPGGGGSCPYSNPSLCGGGGSQLQLQ | 122 |
| CD3ζWT- | ESGPGLVKPSETLSLTCTVSGGSISSSSYYWGWIRQPPGKGLEWIGS | |
| CD28LRM | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYNELNLGRREEYDVLDKRR | |
| | GRDPEMGGKPRRKNPQEGLYNELQKDKMAEAYSEIGMKGERRR | |
| | GKGHDGLYQGLSTATKDTYDALHMQALPPRGGGSYQPYAPPRDF | |
| | AAYRS | |
| 2G1-RSR- | MALPVTALLLPLALLLHAARPGGGGSCPYSNPSLCGGGGSQLQLQ | 123 |
| BB- | ESGPGLVKPSETLSLTCTVSGGSISSSSYYWGWIRQPPGKGLEWIGS | |
| FullCD8Cyto- | IYYSGNIYHNPSLKSRVSISVDTSKNQFSLRLSSVTAADTAVYYCA | |
| CD3ζWT | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITKRGRKKLLYIFKQPFMRPVQTTQEEDGCSCRFPEEEEGGC | |
| | ELLYCNHRNRRRVCKCPRPVVKSGDKPSLSARYVRVKFSRSADAP | |
| | AYQQGQNQLYNELNLGRREEYDVLDKRRGRDPEMGGKPRRKNP | |
| | QEGLYNELQKDKMAEAYSEIGMKGERRRGKGHDGLYQGLSTAT | |
| | KDTYDALHMQALPPR | |
| 2G1-RSR- | MALPVTALLLPLALLLHAARPGGGGSCPYSNPSLCGGGGSQLQLQ | 124 |
| BB- | ESGPGLVKPSETLSLTCTVSGGSISSSSYYWGWIRQPPGKGLEWIGS | |
| 2XCD8LRM- | IYYSGNIYHNPSLKSRVSISVDTSKNQFSLRLSSVTAADTAVYYCA | |
| CD3ζWT | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITKRGRKKLLYIFKQPFMRPVQTTQEEDGCSCRFPEEEEGGC | |
| | ELRRVCKCPRRRVCKCPRRVKFSRSADAPAYQQGQNQLYNELNL | |
| | GRREEYDVLDKRRGRDPEMGGKPRRKNPQEGLYNELQKDKMAE | |
| | AYSEIGMKGERRRGKGHDGLYQGLSTATKDTYDALHMQALPPR | |
| 2G1-RSR- | MALPVTALLLPLALLLHAARPGGGGSCPYSNPSLCGGGGSQLQLQ | 125 |
| CD3ζζ | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYNELNLGRREEYDVLDKRR | |
| | GRDPEMGGKPRRKNPQEGLYNELQKDKMAEAYSEIGMKGERRR | |
| | GKGHDGLYQGLSTATKDTYDALHMQALPPRGRVKFSRSADAPAY | |
| | QQGQNQLYNELNLGRREEYDVLDKRRGRDPEMGGKPRRKNPQE | |
| | GLYNELQKDKMAEAYSEIGMKGERRRGKGHDGLYQGLSTATKD | |
| | TYDALHMQALPPR | |
| 2G1-RSR- | MALPVTALLLPLALLLHAARPGGGGSCPYSNPSLCGGGGSQLQLQ | 126 |
| CD3ζζ | ESGPGLVKPSETLSLTCTVSGGSISSSSYYWGWIRQPPGKGLEWIGS | |
| (YAYAYA) | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYAELNLGRREEYDVLDKRR | |
| | GRDPEMGGKPRRKNPQEGLYAELQKDKMAEAYSEIGMKGERRR | |
| | GKGHDGLYAGLSTATKDTYDALHMQALPPRRVKFSRSADAPAYQ | |
| | QGQNQLYAELNLGRREEYDVLDKRRGRDPEMGGKPRRKNPQEGL | |
| | YAELQKDKMAEAYSEIGMKGERRRGKGHDGLYAGLSTATKDTY | |
| | DALHMQALPPR | |
| 2G1-RSR- | MALPVTALLLPLALLLHAARPGGGGSCPYSNPSLCGGGGSQLQLQ | 127 |
| CD3ζζ | ESGPGLVKPSETLSLTCTVSGGSISSSSYYWGWIRQPPGKGLEWIGS | |
| (zdzezg-6xYA) | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYAELNLGRREEYDVLDKRR | |
| | GRDPEMGGKDTQALLRNDQVYAPLRDRDDAQYSHLGGNKGPRR | |
| | KNPQEGLYAELQKDKMAEAYSEIGMALPPRGRVKFSRSADERPPP | |
| | VPNPDYAPIRKGQRDLYSGLNQRRGRDPEMGGKERRRGKGHDGL | |
| | YAGLSTATKDTYDALHMQKGDKQTLLPNDQLYAPLKDREDDQY | |
| | SHLQGNALPPR | |
| 2G1-RSR- | MALPVTALLLPLALLLHAARPGGGGSCPYSNPSLCGGGGSQLQLQ | 128 |
| CD3ζζ(zdzezg) | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYNELNLGRREEYDVLDKRR | |
| | GRDPEMGGKDTQALLRNDQVYQPLRDRDDAQYSHLGGNKGPRR | |
| | KNPQEGLYNELQKDKMAEAYSEIGMALPPRGRVKFSRSADERPPP | |
| | VPNPDYEPIRKGQRDLYSGLNQRRGRDPEMGGKERRRGKGHDGL | |
| | YQGLSTATKDTYDALHMQKGDKQTLLPNDQLYQPLKDREDDQY | |
| | SHLQGNALPPR | |
| 2G1-RSR- | MALPVTALLLPLALLLHAARPGGGGSCPYSNPSLCGGGGSQLQLQ | 129 |
| CD3ζζ | ESGPGLVKPSETLSLTCTVSGGSISSSSYYWGWIRQPPGKGLEWIGS | |
| (dzezgz-6xYA) | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADDTQALLRNDQVYAPLRDRDDAQYSHLGGNR | |
| | GRDPEMGGKAPAYQQGQNQLYAELNLGRREEYDVLDKRKGERP | |
| | PPVPNPDYAPIRKGQRDLYSGLNQRALPPRGRVKFSRSADPRRKNP | |
| | QEGLYAELQKDKMAEAYSEIGMRGRDPEMGGKDKQTLLPNDQL | |
| | YAPLKDREDDQYSHLQGNKGERRRGKGHDGLYAGLSTATKDTY | |
| | DALHMQALPPR | |
| 2G1-RSR- | MALPVTALLLPLALLLHAARPGGGGSCPYSNPSLCGGGGSQLQLQ | 130 |
| CD3ζζ(dzezgz) | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADDTQALLRNDQVYQPLRDRDDAQYSHLGGNR | |
| | GRDPEMGGKAPAYQQGQNQLYNELNLGRREEYDVLDKRKGERP | |
| | PPVPNPDYEPIRKGQRDLYSGLNQRALPPRGRVKFSRSADPRRKNP | |
| | QEGLYNELQKDKMAEAYSEIGMRGRDPEMGGKDKQTLLPNDQL | |
| | YQPLKDREDDQYSHLQGNKGERRRGKGHDGLYQGLSTATKDTY | |
| | DALHMQALPPR | |
| 2G1-RSR- | MALPVTALLLPLALLLHAARPGGGGSCPYSNPSLCGGGGSQLQLQ | 131 |
| CD3ζζ(degzzz) | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADDTQALLRNDQVYQPLRDRDDAQYSHLGGNR | |
| | GRDPEMGGKERPPPVPNPDYEPIRKGQRDLYSGLNQRKGDKQTLL | |
| | PNDQLYQPLKDREDDQYSHLQGNALPPRGRVKFSRSADAPAYQQ | |
| | GQNQLYNELNLGRREEYDVLDKRRGRDPEMGGKPRRKNPQEGLY | |
| | NELQKDKMAEAYSEIGMKGERRRGKGHDGLYQGLSTATKDTYD | |
| | ALHMQALPPR | |
| 2G1-RSR- | MALPVTALLLPLALLLHAARPGGGGSCPYSNPSLCGGGGSQLQLQ | 132 |
| CD3ζζ(zzzdeg) | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYNELNLGRREEYDVLDKRR | |
| | GRDPEMGGKPRRKNPQEGLYNELQKDKMAEAYSEIGMKGERRR | |
| | GKGHDGLYQGLSTATKDTYDALHMQALPPRGRVKFSRSADDTQA | |
| | LLRNDQVYQPLRDRDDAQYSHLGGNRGRDPEMGGKERPPPVPNP | |
| | DYEPIRKGQRDLYSGLNQRKGDKQTLLPNDQLYQPLKDREDDQY | |
| | SHLQGNALPPR | |
| 2G1-RSR- | MALPVTALLLPLALLLHAARPGGGGSCPYSNPSLCGGGGSQLQLQ | 133 |
| CD3ζ | ESGPGLVKPSETLSLTCTVSGGSISSSSYYWGWIRQPPGKGLEWIGS | |
| (YAYAYA) | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYAELNLGRREEYDVLDKRR | |
| | GRDPEMGGKPRRKNPQEGLYAELQKDKMAEAYSEIGMKGERRR | |
| | GKGHDGLYAGLSTATKDTYDALHMQALPPR | |
| 2G1-RSR- | MALPVTALLLPLALLLHAARPGGGGSCPYSNPSLCGGGGSQLQLQ | 134 |
| CD3ζ(dzz) | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADDTQALLRNDQVYQPLRDRDDAQYSHLGGNR | |
| | GRDPEMGGKPRRKNPQEGLYNELQKDKMAEAYSEIGMKGERRR | |
| | GKGHDGLYQGLSTATKDTYDALHMQALPPR | |
| 2G1-RSR- | MALPVTALLLPLALLLHAARPGGGGSCPYSNPSLCGGGGSQLQLQ | 135 |
| CD3ζ(ezz) | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADERPPPVPNPDYEPIRKGQRDLYSGLNQRRGR | |
| | DPEMGGKPRRKNPQEGLYNELQKDKMAEAYSEIGMKGERRRGK | |
| | GHDGLYQGLSTATKDTYDALHMQALPPR | |
| 2G1-RSR- | MALPVTALLLPLALLLHAARPGGGGSCPYSNPSLCGGGGSQLQLQ | 136 |
| CD3ζ(gzz) | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADDKQTLLPNDQLYQPLKDREDDQYSHLQGNR | |
| | GRDPEMGGKPRRKNPQEGLYNELQKDKMAEAYSEIGMKGERRR | |
| | GKGHDGLYQGLSTATKDTYDALHMQALPPR | |
| 2G1-RSR- | MALPVTALLLPLALLLHAARPGGGGSCPYSNPSLCGGGGSQLQLQ | 137 |
| CD3ζ(deg) | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADDTQALLRNDQVYQPLRDRDDAQYSHLGGNR | |
| | GRDPEMGGKERPPPVPNPDYEPIRKGQRDLYSGLNQRKGDKQTLL | |
| | PNDQLYQPLKDREDDQYSHLQGNALPPR | |
| 2G1-RSR- | MALPVTALLLPLALLLHAARPGGGGSCPYSNPSLCGGGGSQLQLQ | 138 |
| CD3ζYA | ESGPGLVKPSETLSLTCTVSGGSISSSSYYWGWIRQPPGKGLEWIGS | |
| | IYYSGNIYHNPSLKSRVSISVDTSKNQFSLRLSSVTAADTAVYYCA | |
| | REIIVGATHFDYWGQGTLVTVSSGGGGSGGGGSGGGGSGGGGSAI | |
| | QMTQSPSSLSASVGDRVTITCRASQGIRNDLGWYQQKPGKAPELLI | |
| | YAASSLQSGVPSRFSGSGSGTDFTLTISSLQPEDFATYYCLQDYNY | |
| | PLTFGPGTKVDIKGGGGSCPYSNPSLCGGGGSTTTPAPRPPTPAPTI | |
| | ASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEE | |
| | GGCELRVKFSRSADAPAYQQGQNQLYAELNLGRREEYDVLDKRR | |
| | GRDPEMGGKPRRKNPQEGLYNELQKDKMAEAYSEIGMKGERRR | |
| | GKGHDGLYQGLSTATKDTYDALHMQALPPR | |
| 10G1-K-CD8 | MALPVTALLLPLALLLHAARPEVQLLESGGGLVQPGGSLRLSCAA | 139 |
| FL-CD3ζWT | SGFTFSSYAMNWVRQAPGKGLEWVSTISGSGGSTYYADSVKGRFT | |
| | ISRDNSKNTLYLQMNSLRAEDTAVFYCAIDPEYYDILTGGDYWGQ | |
| | GTLVTVSSGGGGSGGGGSGGGGSGGGGSDIQMTQSPSAMSASVG | |
| | DRVTITCRASQGISNYLAWFQQKPGKVPKRLIYAASSLQSGVPSRF | |
| | SGSGSGTEFTLTISSLQPEDFATYFCLQHDSFPLTFGGGTKVEIKTTT | |
| | PAPRPPTPAPTIASQPLSLRPEACRPAAGGAVHTRGLDFACDIYIWA | |
| | PLAGTCGVLLLSLVITLYCNHRNRRRVCKCPRPVVKSGDKPSLSAR | |
| | YVKRGRKKLLYIFKQPFMRPVQTTQEEDGCSCRFPEEEEGGCELR | |
| | VKFSRSADAPAYQQGQNQLYNELNLGRREEYDVLDKRRGRDPEM | |
| | GGKPRRKNPQEGLYNELQKDKMAEAYSEIGMKGERRRGKGHDG | |
| | LYQGLSTATKDTYDALHMQALPPR | |
| 10G1-K- | MALPVTALLLPLALLLHAARPEVQLLESGGGLVQPGGSLRLSCAA | 140 |
| 2XCD8LRM- | SGFTFSSYAMNWVRQAPGKGLEWVSTISGSGGSTYYADSVKGRFT | |
| CD3ζWT | ISRDNSKNTLYLQMNSLRAEDTAVFYCAIDPEYYDILTGGDYWGQ | |
| | GTLVTVSSGGGGSGGGGSGGGGSGGGGSDIQMTQSPSAMSASVG | |
| | DRVTITCRASQGISNYLAWFQQKPGKVPKRLIYAASSLQSGVPSRF | |
| | SGSGSGTEFTLTISSLQPEDFATYFCLQHDSFPLTFGGGTKVEIKTTT | |
| | PAPRPPTPAPTIASQPLSLRPEACRPAAGGAVHTRGLDFACDIYIWA | |
| | PLAGTCGVLLLSLVITRRVCKCPRRRVCKCPRKRGRKKLLYIFKQP | |
| | FMRPVQTTQEEDGCSCRFPEEEEGGCELRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 10G1-K- | MALPVTALLLPLALLLHAARPEVQLLESGGGLVQPGGSLRLSCAA | 141 |
| CD28LRM- | SGFTFSSYAMNWVRQAPGKGLEWVSTISGSGGSTYYADSVKGRFT | |
| CD3ζWT | ISRDNSKNTLYLQMNSLRAEDTAVFYCAIDPEYYDILTGGDYWGQ | |
| | GTLVTVSSGGGGSGGGGSGGGGSGGGGSDIQMTQSPSAMSASVG | |
| | DRVTITCRASQGISNYLAWFQQKPGKVPKRLIYAASSLQSGVPSRF | |
| | SGSGSGTEFTLTISSLQPEDFATYFCLQHDSFPLTFGGGTKVEIKTTT | |
| | PAPRPPTPAPTIASQPLSLRPEACRPAAGGAVHTRGLDFACDIYIWA | |
| | PLAGTCGVLLLSLVITLYCGGGSYQPYAPPRDFAAYRSGGGSKRG | |
| | RKKLLYIFKQPFMRPVQTTQEEDGCSCRFPEEEEGGCELRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| 10G1-K- | MALPVTALLLPLALLLHAARPEVQLLESGGGLVQPGGSLRLSCAA | 142 |
| CD28LRMY3- | SGFTFSSYAMNWVRQAPGKGLEWVSTISGSGGSTYYADSVKGRFT | |
| CD3ζWT | ISRDNSKNTLYLQMNSLRAEDTAVFYCAIDPEYYDILTGGDYWGQ | |
| | GTLVTVSSGGGGSGGGGSGGGGSGGGGSDIQMTQSPSAMSASVG | |
| | DRVTITCRASQGISNYLAWFQQKPGKVPKRLIYAASSLQSGVPSRF | |
| | SGSGSGTEFTLTISSLQPEDFATYFCLQHDSFPLTFGGGTKVEIKTTT | |
| | PAPRPPTPAPTIASQPLSLRPEACRPAAGGAVHTRGLDFACDIYIWA | |
| | PLAGTCGVLLLSLVITLYCGGGSFQPFAPPRDFAAFRSGGGSKRGR | |
| | KKLLYIFKQPFMRPVQTTQEEDGCSCRFPEEEEGGCELRVKFSRSA | |
| | DAPAYQQGQNQLYNELNLGRREEYDVLDKRRGRDPEMGGKPRR | |
| | KNPQEGLYNELQKDKMAEAYSEIGMKGERRRGKGHDGLYQGLST | |
| | ATKDTYDALHMQALPPR | |
| 10G1-K- | MALPVTALLLPLALLLHAARPEVQLLESGGGLVQPGGSLRLSCAA | 143 |
| CD3ζWT- | SGFTFSSYAMNWVRQAPGKGLEWVSTISGSGGSTYYADSVKGRFT | |
| CD8LRM | ISRDNSKNTLYLQMNSLRAEDTAVFYCAIDPEYYDILTGGDYWGQ | |
| | GTLVTVSSGGGGSGGGGSGGGGSGGGGSDIQMTQSPSAMSASVG | |
| | DRVTITCRASQGISNYLAWFQQKPGKVPKRLIYAASSLQSGVPSRF | |
| | SGSGSGTEFTLTISSLQPEDFATYFCLQHDSFPLTFGGGTKVEIKTTT | |
| | PAPRPPTPAPTIASQPLSLRPEACRPAAGGAVHTRGLDFACDIYIWA | |
| | PLAGTCGVLLLSLVITLYCKRGRKKLLYIFKQPFMRPVQTTQEEDG | |
| | CSCRFPEEEEGGCELRVKFSRSADAPAYQQGQNQLYNELNLGRRE | |
| | EYDVLDKRRGRDPEMGGKPRRKNPQEGLYNELQKDKMAEAYSEI | |
| | GMKGERRRGKGHDGLYQGLSTATKDTYDALHMQALPPRGGGGS | |
| | RVCKCPRPV | |
| 10G1-K- | MALPVTALLLPLALLLHAARPEVQLLESGGGLVQPGGSLRLSCAA | 144 |
| CD3ζWT- | SGFTFSSYAMNWVRQAPGKGLEWVSTISGSGGSTYYADSVKGRFT | |
| CD28LRMY3 | ISRDNSKNTLYLQMNSLRAEDTAVFYCAIDPEYYDILTGGDYWGQ | |
| | GTLVTVSSGGGGSGGGGSGGGGSGGGGSDIQMTQSPSAMSASVG | |
| | DRVTITCRASQGISNYLAWFQQKPGKVPKRLIYAASSLQSGVPSRF | |
| | SGSGSGTEFTLTISSLQPEDFATYFCLQHDSFPLTFGGGTKVEIKTTT | |
| | PAPRPPTPAPTIASQPLSLRPEACRPAAGGAVHTRGLDFACDIYIWA | |
| | PLAGTCGVLLLSLVITLYCKRGRKKLLYIFKQPFMRPVQTTQEEDG | |
| | CSCRFPEEEEGGCELRVKFSRSADAPAYQQGQNQLYNELNLGRRE | |
| | EYDVLDKRRGRDPEMGGKPRRKNPQEGLYNELQKDKMAEAYSEI | |
| | GMKGERRRGKGHDGLYQGLSTATKDTYDALHMQALPPRGGGSF | |
| | QPFAPPRDFAAFRS | |
| 10G1-K-BB- | MALPVTALLLPLALLLHAARPEVQLLESGGGLVQPGGSLRLSCAA | 145 |
| FullCD8Cyto- | SGFTFSSYAMNWVRQAPGKGLEWVSTISGSGGSTYYADSVKGRFT | |
| CD3ζWT | ISRDNSKNTLYLQMNSLRAEDTAVFYCAIDPEYYDILTGGDYWGQ | |
| | GTLVTVSSGGGGSGGGGSGGGGSGGGGSDIQMTQSPSAMSASVG | |
| | DRVTITCRASQGISNYLAWFQQKPGKVPKRLIYAASSLQSGVPSRF | |
| | SGSGSGTEFTLTISSLQPEDFATYFCLQHDSFPLTFGGGTKVEIKTTT | |
| | PAPRPPTPAPTIASQPLSLRPEACRPAAGGAVHTRGLDFACDIYIWA | |
| | PLAGTCGVLLLSLVITKRGRKKLLYIFKQPFMRPVQTTQEEDGCSC | |
| | RFPEEEEGGCELLYCNHRNRRRVCKCPRPVVKSGDKPSLSARYVR | |
| | VKFSRSADAPAYQQGQNQLYNELNLGRREEYDVLDKRRGRDPEM | |
| | GGKPRRKNPQEGLYNELQKDKMAEAYSEIGMKGERRRGKGHDG | |
| | LYQGLSTATKDTYDALHMQALPPR | |
| 10G1-K-BB- | MALPVTALLLPLALLLHAARPEVQLLESGGGLVQPGGSLRLSCAA | 146 |
| 2XCD8LRM- | SGFTFSSYAMNWVRQAPGKGLEWVSTISGSGGSTYYADSVKGRFT | |
| CD3ζWT | ISRDNSKNTLYLQMNSLRAEDTAVFYCAIDPEYYDILTGGDYWGQ | |
| | GTLVTVSSGGGGSGGGGSGGGGSGGGGSDIQMTQSPSAMSASVG | |
| | DRVTITCRASQGISNYLAWFQQKPGKVPKRLIYAASSLQSGVPSRF | |
| | SGSGSGTEFTLTISSLQPEDFATYFCLQHDSFPLTFGGGTKVEIKTTT | |
| | PAPRPPTPAPTIASQPLSLRPEACRPAAGGAVHTRGLDFACDIYIWA | |
| | PLAGTCGVLLLSLVITKRGRKKLLYIFKQPFMRPVQTTQEEDGCSC | |
| | RFPEEEEGGCELRRVCKCPRRRVCKCPRRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |

The recombinant antigen receptors of the present invention can be used to: (a) treat, prevent, ameliorate one or more symptoms of a condition associated with malignant cells expressing in a subject an antigen that the recombinant antigen receptor binds to (e.g., cancer); (b) inhibit tumor growth or progression in a subject (who has a malignant tumor expressing an antigen that the recombinant antigen receptor binds to); (c) inhibit metastasis of cancer (malignant) cells expressing an antigen that the recombinant antigen receptor binds to in a subject (who has one or more malignant cells expressing such an antigen); (d) induce regression (e.g., long-term regression) of a tumor expressing an antigen that the recombinant antigen receptor binds to; (e) exert cytotoxic activity in malignant cells expressing an antigen that the recombinant antigen receptor binds to; (f) block interaction between such an antigen and a factor yet to be identified; and/or (g) induce a bystander effect that kills or inhibits growth of malignant cells in the vicinity that do not express such an antigen.

The recombinant antigen receptors provided herein can encompass monoclonal antibodies, polyclonal antibodies, antibody fragments (e.g., Fab, Fab′, F(ab′)2, Fv, Fc, etc.), chimeric antibodies, single chain (ScFv), and/or humanized antibodies. The antibodies may be murine, rat, human, or any other origin (including chimeric or humanized antibodies).

The invention further provides a polynucleotide comprising a DNA sequence encoding the recombinant antigen receptor described herein. The invention further provides a vector comprising the polynucleotide. The invention further provides an engineered immune cell that comprises the polynucleotide and/or comprises the vector. The invention further provides an engineered immune cell comprising a first recombinant antigen receptor as described herein. Conventional molecular biology techniques may be used to produce these.

Additional methods to improve signalling by the recombinant antigen receptor.

In another aspect, the invention further provides an engineered immune cell comprising a first recombinant antigen receptor as described herein and further comprising a second recombinant antigen receptor comprising an extracellular antigen binding domain and an intracellular domain that comprises a Lck, LAT, ZAP70, or a variant thereof, e.g. a functional variant thereof. In an embodiment of this engineered immune cell, the antigen binding domain of the second recombinant antigen receptor binds to the same antigen as the first recombinant antigen receptor or the antigen binding domain of the second recombinant antigen receptor binds to a different antigen from the first recombinant antigen receptor. For example, the antigen binding domain of the first recombinant antigen receptor may bind to DLL3 on a target tumor cell, while the antigen binding domain of the second recombinant antigen receptor may bind to a second, different tumor antigen.

In an embodiment, the immune cell is a T cell. In an embodiment, the immune cell comprises one or more genomic modifications to the TCRa (TCR alpha) gene. In an embodiment, the immune cell expresses an exogenous downstream mediator of T cell signaling. In an embodiment, the exogenous downstream mediator of T cell signaling is one or more of ZAP70, Lck, Fyn, Syk, LAT or Unc119 (e.g. the downstream mediator comprises the amino acid sequence of SEQ ID NOs: 1 (ZAP70), 2 (Lck), 4 (Fyn), 6 (Syk), 8 (LAT), or 9 (Unc119)), or a variant thereof (e.g. the downstream mediator comprises the amino acid sequence of SEQ ID NOs: 3 (Truncated Lck), 5 (Truncated Fyn), or 7 (Truncated Syk)). In an embodiment, the antigen binding domain of the recombinant antigen receptor binds to an antigen characteristic of a disease or condition e.g. cancer. In an embodiment, the antigen binding domain of the recombinant antigen receptor binds to DLL3.

In another aspect, the invention further provides an engineered immune cell comprising a CAR and expressing one or more exogenous downstream mediators of T cell signaling. In an embodiment, the exogenous downstream mediator of T cell signaling is one or more of ZAP70, Lck, Fyn, Syk, LAT or Unc119 (e.g. the downstream mediator comprises the amino acid sequence of SEQ ID NOs: 1 (ZAP70), 2 (Lck), 4 (Fyn), 6 (Syk), 8 (LAT), or 9 (Unc119)), or a variant thereof, e.g. a functional variant thereof (e.g. the downstream mediator comprises the amino acid sequence of SEQ ID NOs: 3 (Truncated Lck), 5 (Truncated Fyn), or 7 (Truncated Syk)). In an embodiment, the immune cell is a T cell. In an embodiment, the immune cell comprises one or more genomic modifications to the TCRa (TCR alpha) gene. In an embodiment, the antigen binding domain of the recombinant antigen receptor binds to an antigen characteristic of a disease or condition e.g. cancer. In an embodiment, the antigen binding domain of the recombinant antigen receptor binds to DLL3.

In a further aspect, the invention provides an engineered immune cell comprising a first recombinant antigen receptor and a second recombinant antigen receptor, wherein the first recombinant antigen receptor comprises a CAR and the second recombinant antigen receptor comprises an extracellular antigen binding domain and an intracellular domain that comprises Lck, LAT, ZAP70, or a variant thereof. In an embodiment, the antigen binding domain of the second recombinant antigen receptor binds to the same antigen as the first recombinant antigen receptor or the antigen binding domain of the second recombinant antigen receptor binds to a different antigen from the first recombinant antigen receptor. In an embodiment, the immune cell is a T cell. In an embodiment, the immune cell comprises one or more genomic modifications to the TCRa (TCR alpha) gene. In an embodiment, the immune cell expresses an exogenous downstream mediator of T cell signaling. In an embodiment, the exogenous downstream mediator of T cell signaling is one or more of ZAP70, Lck, Fyn, Syk, LAT or Unc119 (e.g. the downstream mediator comprises the amino acid sequence of SEQ ID NOs: 1 (ZAP70), 2 (Lck), 4 (Fyn), 6 (Syk), 8 (LAT), or 9 (Unc119)), or a variant thereof, e.g. a functional variant thereof (e.g. the downstream mediator comprises the amino acid sequence of SEQ ID NOs: 3 (Truncated Lck), 5 (Truncated Fyn), or 7 (Truncated Syk)). In an embodiment, the antigen binding domain of the recombinant antigen receptor binds to an antigen characteristic of a disease or condition e.g. cancer. In an embodiment, the antigen binding domain of the recombinant antigen receptor binds to DLL3.

In a further aspect, the invention provides a pharmaceutical composition comprising any of the engineered immune cells described in this section. In a further aspect, the invention provides a method of treating cancer comprising administering to a patient in need thereof an effective amount of the pharmaceutical composition or an effective amount of any of the engineered immune cells described in this section.

Improved Recombinant Antigen Receptors e.g. Improved CARs and Methods of Making Thereof

Provided herein are improved recombinant antigen receptors, e.g. improved CARs. CARs provided herein include single chain CARS and multichain CARs. The CARs have the ability to redirect T cell specificity and reactivity toward the antigen they bind e.g. DLL3 in a non-MHC-restricted manner, exploiting the antigen-binding properties of monoclonal antibodies. The non-MHC-restricted antigen recognition gives T cells expressing CARs the ability to recognize an antigen independent of antigen processing, thus bypassing a major mechanism of tumor escape.

In some embodiments, CARs provided herein comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain.

In some embodiments, the extracellular ligand-binding domain of the recombinant antigen receptor of the invention, e.g. CAR of the invention, comprises an scFv comprising the light chain variable (VL) region and the heavy chain variable (VH) region of a target antigen specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)₃ (SEQ ID NO: 147), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides, e.g. comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The invention encompasses modifications to the CARs and polypeptides of the invention shown in Table 2, including functionally equivalent CARs having modifications which do not significantly affect their properties and variants which have enhanced or decreased activity and/or affinity. For example, the amino acid sequence may be mutated to obtain an antibody with the desired binding affinity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 4 under the heading of “conservative substitutions.” If such substitutions result in a change in biological activity, then more substantial changes, denominated “exemplary substitutions” in Table 4, or as further described below in reference to amino acid classes, may be introduced and the products screened.

TABLE 2
Amino Acid Substitutions

| Original | | |
|---|---|---|
| Residue | | |
| (naturally | | |
| occurring | | |
| amino | Conservative | Exemplary |
| acid) | Substitutions | Substitutions |
| Ala (A) | Val | Val; Leu; Ile |
| Arg (R) | Lys | Lys; Gln; Asn |
| Asn (N) | Gln | Gln; His; Asp, |
| | | Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; |
| | | Arg |
| Ile (I) | Leu | Leu; Val; Met; |
| | | Ala; Phe; |
| | | Norleucine |
| Leu (L) | Ile | Norleucine; Ile; |
| | | Val; Met; Ala; |
| | | Phe |
| Lys (K) | Arg | Arg; Gln; Asn |
| Met (M) | Leu | Leu; Phe; Ile |
| Phe (F) | Tyr | Leu; Val; Ile; |
| | | Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; |
| | | Thr; Ser |
| Val (V) | Leu | Ile; Leu; Met; |
| | | Phe; Ala; |
| | | Norleucine |

The binding affinity (K_{D}) of the ligand binding domain of the recombinant antigen receptor of the present invention e.g a CAR of the present invention as described herein to its target antigen e.g. DLL3 (such as human DLL3) can be for example about 0.1 to about 1000 nM, for example between about 0.5 nM to about 500 nM, or for example between about 1 nM to about 250 nM. In some embodiments, the binding affinity is about any of 1000 nm, 750 nm, 500 nm, 400 nm, 300 nm, 250 nm, 200 nM, 100 nM, 90 nM, 80 nM, 70 nM, 60 nM, 50 nM, 45 nM, 40 nM, 35 nM, 30 nM, 25 nM, 20 nM, 19 nm, 18 nm, 17 nm, 16 nm, 15 nM, 10 nM, 8 nM, 7.5 nM, 7 nM, 6.5 nM, 6 nM, 5.5 nM, 5 nM, 4 nM, 3 nM, 2 nM, 1 nM, 0.5 nM, 0.3 nM or 0.1 nM.

In some embodiments, the binding affinity (K_{D}) of the scFv of the ligand binding domain of the recombinant antigen receptor as described herein to its target antigen e.g. DLL3 is about 10 nM to about 100 nM, about 10 nM to about 90 nM, about 10 nM to about 80 nM, about 20 nM to about 70 nM, about 25 nM to about 75 nM, or about 40 nM to about 110 nM. In one embodiment, the binding affinities of the scFv described in this paragraph are for human DLL3.

In some embodiments, the binding affinity is less than about any of 1000 nm, 900 nm, 800 nm, 250 nM, 200 nM, 100 nM, 50 nM, 30 nM, 20 nM, 10 nM, 7.5 nM, 7 nM, 6.5 nM, 6 nM, 5 nM.

### Monoclonal Antibody-Specific Epitopes

In some embodiments, the extracellular domain of any one of the improved recombinant antigen receptors disclosed herein may comprise one or more epitopes specific for (i.e., specifically recognized by) a monoclonal antibody. These epitopes are also referred to herein as mAb-specific epitopes. In these embodiments, the extracellular domain comprises the VH and VL polypeptides that specifically bind to the target antigen of choice e.g. DLL3 and one or more epitopes that bind to one or more monoclonal antibodies (mAbs). CARs comprising the mAb-specific epitopes can be single-chain or multi-chain.

The inclusion of epitopes specific for monoclonal antibodies in the extracellular domain of the CARs described herein allows sorting and depletion of engineered immune cells expressing the CARs. In some embodiments, this feature also promotes recovery of endogenous target antigen-expressing cells that were depleted by administration of engineered immune cells expressing the CARs.

Accordingly, in some embodiments, the present invention relates to a method for sorting and/or depleting the engineered immune cells endowed with the CARs comprising mAb-specific epitopes and a method for promoting recovery of endogenous target-antigen-expressing cells, such as bone marrow progenitor cells.

Several epitope-monoclonal antibody couples can be used to generate CARs comprising monoclonal antibody specific epitopes; in particular, those already approved for medical use, such as CD20 epitope/rituximab as a non-limiting example.

In some embodiments, the monoclonal antibody specific for the epitope may be conjugated with a cytotoxic drug. It is also possible to promote CDC cytotoxicity by using engineered antibodies on which are grafted component(s) of the complement system. In some embodiments, activation of the CAR-T cells can be modulated by depleting the cells using an antibody which recognizes the epitope.

The invention also encompasses methods for sorting the engineered immune cells endowed with the target-antigen-specific recombinant antigen receptors e.g. CARs expressing the mAb-specific epitope(s) and therapeutic methods where the activation of the engineered immune cells endowed with these target-antigen-specific recombinant antigen receptors is modulated by depleting the cells using an antibody that targets the external ligand binding domain of said CARs.

CARs comprising one or more epitopes specifically recognized by a monoclonal antibody are disclosed in WO2016/120216, which is hereby incorporated by reference in its entirety. The epitope can be selected from any number of epitopes known in the art. In some embodiments, the epitope can be a target of a monoclonal antibody approved for medical use, such as, for example without limitation, the CD20 epitope recognized by rituximab.

In some embodiments, the epitope can be located between the scFv and the hinge of a CAR. In some embodiments, two instances of the same epitope, separated by linkers, may be used in the CAR.

In some embodiments, the extracellular binding domain of the CAR comprising the VH and VL polypeptides and the mAb-specific epitope(s) may have different structures depending on the position of insertion of the epitope and the use of linkers. For example, the extracellular binding domain of the recombinant antigen receptor e.g. CAR of the present invention comprising mAb-specific epitopes may have one of the following structures:
V₁-L₁-V₂-(L)ₓ-Epitope1-(L)ₓ-;
V₁-L₁-V₂-(L)ₓ-Epitope1-(L)ₓ-Epitope2-(L)ₓ-;
V₁-L₁-V₂-(L)ₓ-Epitope1-(L)ₓ-Epitope2-(L)ₓ-Epitope3-(L)ₓ-;
(L)ₓ-Epitope1-(L)ₓ-V₁-L₁-V₂;
(L)ₓ-Epitope1-(L)ₓ-Epitope2-(L)ₓ-V₁-L₁-V₂;
Epitope1-(L)ₓ-Epitope2-(L)ₓ-Epitope3-(L)ₓ-V₁-L₁-V₂;
(L)ₓ-Epitope1-(L)ₓ-V₁-L₁-V₂-(L)ₓ-Epitope2-(L)ₓ;
(L)ₓ-Epitope1-(L)ₓ-V₁-L₁-V₂-(L)ₓ-Epitope2-(L)ₓ-Epitope3-(L)ₓ-;
(L)ₓ-Epitope1-(L)ₓ-V₁-L₁-V₂-(L)ₓ-Epitope2-(L)ₓ-Epitope3-(L)ₓ-Epitope4-(L)ₓ-;
(L)ₓ-Epitope1-(L)ₓ-Epitope2-(L)ₓ-V₁-L₁-V₂-(L)ₓ-Epitope3-(L)ₓ-;
(L)ₓ-Epitope1-(L)ₓ-Epitope2-(L)ₓ-V₁-L₁-V₂-(L)ₓ-Epitope3-(L)ₓ-Epitope4-(L)ₓ-;
V₁-(L)ₓ-Epitope1-(L)ₓ-V₂;
V₁-(L)ₓ-Epitope1-(L)ₓ-V₂-(L)ₓ-Epitope2-(L)ₓ;
V₁-(L)ₓ-Epitope1-(L)ₓ-V₂-(L)ₓ-Epitope2-(L)ₓ-Epitope3-(L)ₓ;
V₁-(L)ₓ-Epitope1-(L)ₓ-V₂-(L)ₓ-Epitope2-(L)ₓ-Epitope3-(L)ₓ-Epitope4-(L)ₓ;
(L)ₓ-Epitope1-(L)ₓ-V₁-(L)ₓ-Epitope2-(L)ₓ-V₂; or,
(L)ₓ-Epitope1-(L)ₓ-V₁-(L)ₓ-Epitope2-(L)ₓ-V₂-(L)ₓ-Epitope3-(L)ₓ;
wherein,
V₁ is V_{L} and V₂ is V_{H} or V₁ is V_{H} and V₂ is V_{L};
L₁ is a linker suitable to link the V_{H} chain to the V_{L} chain;
L is a linker comprising glycine and serine residues, and each occurrence of L in the extracellular binding domain can be identical or different to other occurrence of L in the same extracellular binding domain, for example SGGGG (SEQ ID NO:148), GGGGS (SEQ ID NO:52) or SGGGGS (SEQ ID NO:149), and,
x is 0 or 1 and each occurrence of x is selected independently from the others; and,
Epitope 1, Epitope 2, Epitope 3 and Epitope 4 are mAb-specific epitopes and can be identical or different. In some embodiments, Epitope 1, Epitope 2 and Epitope 4 are a mAb-specific epitope having a first amino acid sequence and Epitope 3 is a mAb-specific epitope having a second amino acid sequence.

In some embodiments, the extracellular binding domain of the CAR comprises the following sequence
V₁-L₁-V₂-(L)ₓ-Epitope1-(L)ₓ-Epitope2-(L)ₓ-; or,
(L)ₓ-Epitope1-(L)ₓ-V₁-L₁-V₂-(L)ₓ-Epitope2-(L)ₓ-Epitope3-(L)ₓ-Epitope4-(L)ₓ-. wherein V₁, V₂, L₁, L, x and Epitope 1, Epitope 2, Epitope 3 and Epitope 4 are as defined above.

In some embodiments, any one of the recombinant antigen receptors e.g. CARs disclosed herein may comprise one or more mAb-specific epitopes selected from a CD52 epitope, a CD20 epitope, a CD3 epitope, a CD41 epitope, a CD25 epitope, a CD30 epitope, an EGFR epitope, a TNFα epitope, a VEGF epitope, a complement protein C5 epitope, a CD11a epitope, a CD33 epitope, an alpha-4 integrin epitope, an IgE Fc region epitope, an RSV protein F epitope, an IL-6 receptor epitope, a HER2 receptor epitope, an integrin α₄β₇ epitope, a BAFF (B-cell activatin factor) epitope, an IL-1β epitope, a RANKL epitope, a CTLA4 epitope, a CD34 epitope, an IL-12 epitope, and/or an IL-23 epitope.

In some embodiments, the recombinant antigen receptors e.g. CARs disclosed herein may comprise one or more mAb-specific epitopes selected from epitopes specifically recognized by alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and/or ustekinumab.

In some embodiments, the recombinant antigen receptors e.g. CARs comprise one or more mAb-specific epitopes selected from the epitopes disclosed in Table 3.

TABLE 3
Examples of mAb-specific epitopes that can be
used in the extracellular binding domain of the
target-specific CAR of the invention such
as for example mimotopes and epitope with
their corresponding mAb.
Rituximab

| | | |
|---|---|---|
| Mimotope | SEQ ID NO: 81 | CPYSNPSLC |

Palivizumab

| | | |
|---|---|---|
| Epitope | SEQ ID NO: 82 | NSELLSLINDMPITNDQKKLMSNN |

Cetuximab

| | | |
|---|---|---|
| Mimotope 1 | SEQ ID NO: 83 | CQFDLSTRRLKC |
| Mimotope 2 | SEQ ID NO: 84 | CQYNLSSRALKC |
| Mimotope 3 | SEQ ID NO: 85 | CVWQRWQKSYVC |
| Mimotope 4 | SEQ ID NO: 86 | CMWDRFSRWYKC |

Nivolumab

| | | |
|---|---|---|
| Epitope 1 | SEQ ID NO: 87 | SFVLNWYRMSPSNQTDKLAAFPEDR |
| Epitope 2 | SEQ ID NO: 88 | SGTYLCGAISLAPKAQIKE |

QBEND-10

| | | |
|---|---|---|
| Epitope | SEQ ID NO: 89 | ELPTQGTFSNVSTNVSPAKPTTTA |

Alemtuzumab

| | | |
|---|---|---|
| Epitope | SEQ ID NO: 90 | GQNDTSQTSSPS |

The recombinant antigen receptors e.g. CARs of the invention are expressed on the surface membrane of the cell. Thus, the CAR can comprise a transmembrane domain. Suitable transmembrane domains for a CAR disclosed herein have the ability to (a) be expressed at the surface of a cell, e.g. an immune cell such as, for example without limitation, lymphocyte cells or Natural killer (NK) cells, and (b) interact with the ligand-binding domain and intracellular signaling domain for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T cell receptor such as α, β, γ or δ, polypeptide constituting CD3 complex, IL-2 receptor p55 (a chain), p75 (β chain) or γ chain, subunit chain of Fc receptors, in particular Fcγ receptor III, or CD proteins. Alternatively, the transmembrane domain can be synthetic and can comprise predominantly hydrophobic residues such as leucine and valine. In some embodiments said transmembrane domain is derived from the human CD8a chain (e.g., NP_001139345.1).

The transmembrane domain is linked to the extracellular ligand-binding domain by a stalk domain (also called hinge domain). A stalk domain may comprise up to 300 amino acids, e.g. 10 to 100 amino acids or 25 to 50 amino acids. Stalk region may be derived from all or part of naturally occurring molecules, such as from all or part of the extracellular region of CD8, CD4, or CD28, or from all or part of an antibody constant region. Alternatively the stalk domain may be a synthetic sequence that corresponds to a naturally occurring stalk sequence, or may be an entirely synthetic stalk sequence. In some embodiments said stalk domain is a part of human CD8a chain (e.g., NP_001139345.1). In some embodiments, the stalk domain of recombinant antigen receptors e.g. CARs described herein comprises a CD8a hinge, an IgG1 hinge, or an FcγRIIIα hinge. In some embodiments, the stalk domain comprises a human CD8a hinge, a human IgG1 hinge, or a human FcγRIIIα hinge. In some embodiments, CARs disclosed herein can comprise an extracellular ligand-binding domain that specifically binds DLL3, a CD8a human hinge and transmembrane domains, the modified CD3ζ signaling domain as described herein, and 4-1BB co-stimulatory domain.

Downregulation or mutation of target antigens is commonly observed in cancer cells, creating antigen-loss escape variants. Thus, to offset tumor escape and render the immune cell more specific to target, the recombinant antigen receptor e.g. CAR of the invention can comprise one or more additional extracellular ligand-binding domains, to simultaneously bind different elements in target thereby augmenting immune cell activation and function. In some embodiments, the extracellular ligand-binding domains can be placed in tandem on the same transmembrane polypeptide, and optionally can be separated by a linker. In some embodiments, said different extracellular ligand-binding domains can be placed on different transmembrane polypeptides composing the recombinant antigen receptor e.g. CAR. In some embodiments, the invention relates to a population of recombinant antigen receptors, each comprising a different extracellular ligand-binding domain. In particular, the invention relates to a method of engineering immune cells comprising providing an immune cell and expressing at the surface of the cell a population of CARs, each CAR comprising different extracellular ligand-binding domains. In another particular embodiment, the invention relates to a method of engineering an immune cell comprising providing an immune cell and introducing into the cell polynucleotides encoding polypeptides composing a population of CAR each one comprising different extracellular ligand-binding domains. By population of CARs, it is meant at least two, three, four, five, six or more CARs each one comprising different extracellular ligand-binding domains. The different extracellular ligand-binding domains according to the invention can for example simultaneously bind different elements in the target thereby augmenting immune cell activation and function. The invention also relates to an isolated immune cell which comprises a population of CARs each one comprising different extracellular ligand-binding domains.

In another aspect, provided herein are polynucleotides encoding any of the recombinant antigen receptors e.g. CARs and polypeptides described herein. Polynucleotides can be made and expressed by procedures known in the art.

In another aspect, provided herein are compositions (such as a pharmaceutical compositions) comprising any of the cells of the invention. In some embodiments, the composition comprises a cell comprising a polynucleotide encoding any of the CARs described herein.

Expression vectors, and administration of polynucleotide compositions are further described herein.

In another aspect, provided herein is a method of making any of the polynucleotides described herein.

Polynucleotides complementary to any such sequences are also encompassed by the invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the invention, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants generally exhibit at least about 70% identity, or at least about 80% identity, or even at least about 90% or 95% or greater than 95% identity e.g. at least 99% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

Generally, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the invention. Further, alleles of the genes comprising the polynucleotide sequences provided herein are within the scope of the invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

A polynucleotide encoding a recombinant antigen receptor e.g. CAR disclosed herein may exist in an expression cassette or expression vector (e.g., a plasmid for introduction into a bacterial host cell, or a viral vector such as a baculovirus vector for transfection of an insect host cell, or a plasmid or viral vector such as a lentivirus for transfection of a mammalian host cell). In some embodiments, a polynucleotide or vector can include a nucleic acid sequence encoding ribosomal skip sequences such as, for example without limitation, a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see (Donnelly and Elliott 2001; Atkins, Wills et al. 2007; Doronina, Wu et al. 2008)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct transmembrane polypeptides into the secretory pathway of a host cell, in some embodiments, a secretory signal sequence (also known as a leader sequence, prepro sequence or pre sequence) is provided in a polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). In some embodiments the signal peptide comprises the amino acid sequence MALPVTALLLPLALLLHAARP (SEQ ID NO: 150) (CD8 signal sequence) or MIPAVVLLLLLLVEQAAA (SEQ ID NO: 151) (FcεRIγ-signal peptide). Those skilled in the art will recognize that, in view of the degeneracy of the genetic code, considerable sequence variation is possible among these polynucleotide molecules. In some embodiments, nucleic acid sequences of the invention are codon-optimized for expression in mammalian cells, e.g. for expression in primate (e.g. human or monkey) cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

### Methods of Engineering an Immune Cell

Methods of preparing immune cells for use in immunotherapy are provided herein. In some embodiments, the methods comprise obtaining immune cells, introducing a recombinant antigen receptor e.g. CAR according to the invention into immune cells, and expanding the cells. In some embodiments, the invention relates to a method of engineering an immune cell comprising: providing a cell and expressing at the surface of the cell at least one CAR as described herein. Methods for engineering immune cells are described in, for example, PCT Patent Application Publication Nos. WO/2014/039523, WO/2014/184741, WO/2014/191128, WO/2014/184744, and WO/2014/184143, each of which is incorporated herein by reference in its entirety. In some embodiments, the method comprises: transfecting the cell with at least one polynucleotide encoding CAR as described herein, and expressing the polynucleotides in the cell.

Prior to engineering of cells, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available and known to those skilled in the art, may be used. In some embodiments, cells can be derived from a healthy donor or from a donor suffering from a disease or disorder, for example, an individual diagnosed with cancer or from an individual diagnosed with an infection. In some embodiments, cells can be part of a mixed population of cells which present different phenotypic characteristics.

In some embodiments, the polynucleotides are present in lentiviral vectors for stable expression in the cells.

In some embodiments, the method can further comprise a step of genetically modifying a cell by disrupting or inactivating at least one gene expressing, for example without limitation, a component of the TCR, a target for an immunosuppressive agent, an HLA gene, and/or an immune checkpoint protein such as, for example, PDCD1 or CTLA-4. By disruption or inactivating a gene it is intended that the gene of interest is not expressed in a functional protein form. In some embodiments, the gene to be disrupted or inactivated is selected from the group consisting of, for example without limitation, TCRα, TCRβ, CD52, glucocorticoid receptor (GR), deoxycytidine kinase (DCK), PD-1, and CTLA-4. In some embodiments, the method comprises inactivating one or more genes by introducing into the cells a rare-cutting endonuclease able to selectively inactivate a gene by selective DNA cleavage. In some embodiments the rare-cutting endonuclease can be, for example, a transcription activator-like effector nuclease (TALE-nuclease) or Cas9 endonuclease.

In some embodiments, an additional catalytic domain is used with a rare-cutting endonuclease to enhance its capacity to inactivate targeted genes. For example, an additional catalytic domain can be a DNA end-processing enzyme. Non-limiting examples of DNA end-processing enzymes include 5-3′ exonucleases, 3-5′ exonucleases, 5-3′ alkaline exonucleases, 5′ flap endonucleases, helicases, hosphatase, hydrolases and template-independent DNA polymerases. Non-limiting examples of such catalytic domain comprise of a protein domain or catalytically active derivate of the protein domain selected from the group consisting of hExoI (EXO1_HUMAN), Yeast ExoI (EXO1_YEAST), E. coli ExoI, Human TREX2, Mouse TREX1, Human TREX1, Bovine TREX1, Rat TREX1, TdT (terminal deoxynucleotidyl transferase) Human DNA2, Yeast DNA2 (DNA2_YEAST). In some embodiments, an additional catalytic domain can have a 3′-5′-exonuclease activity, and In some embodiments, said additional catalytic domain is TREX, e.g. a TREX2 catalytic domain (WO2012/058458). In some embodiments, said catalytic domain is encoded by a single chain TREX polypeptide. The additional catalytic domain may be fused to a nuclease fusion protein or chimeric protein. In some embodiments, the additional catalytic domain is fused using, for example, a peptide linker.

In some embodiments, the method further comprises a step of introducing into cells an exogeneous nucleic acid comprising at least a sequence homologous to a portion of the target nucleic acid sequence, such that homologous recombination occurs between the target nucleic acid sequence and the exogeneous nucleic acid. In some embodiments, said exogenous nucleic acid comprises first and second portions which are homologous to region 5′ and 3′ of the target nucleic acid sequence, respectively. The exogenous nucleic acid may also comprise a third portion positioned between the first and the second portion which comprises no homology with the regions 5′ and 3′ of the target nucleic acid sequence. Following cleavage of the target nucleic acid sequence, a homologous recombination event is stimulated between the target nucleic acid sequence and the exogenous nucleic acid. In some embodiments, homologous sequences of at least about 50 bp, greater than about 100 bp, or greater than about 200 bp can be used within the donor matrix. The exogenous nucleic acid can be, for example without limitation, from about 200 bp to about 6000 bp, e.g. from about 1000 bp to about 2000 bp. Shared nucleic acid homologies are located in regions flanking upstream and downstream the site of the break, and the nucleic acid sequence to be introduced is located between the two arms.

In some embodiments, a nucleic acid successively comprises a first region of homology to sequences upstream of said cleavage; a sequence to inactivate a targeted gene selected from the group consisting of TCRα, TCRβ, CD52, glucocorticoid receptor (GR), deoxycytidine kinase (DCK), and an immune checkpoint protein such as for example programmed death-1 (PD-1); and a second region of homology to sequences downstream of the cleavage. The polynucleotide introduction step can be simultaneous, before or after the introduction or expression of the rare-cutting endonuclease. Depending on the location of the target nucleic acid sequence wherein break event has occurred, such exogenous nucleic acid can be used to knock-out a gene, e.g. when exogenous nucleic acid is located within the open reading frame of the gene, or to introduce new sequences or genes of interest. Sequence insertions by using such exogenous nucleic acid can be used to modify a targeted existing gene, by correction or replacement of the gene (allele swap as a non-limiting example), or to up- or down-regulate the expression of the targeted gene (promoter swap as non-limiting example), the targeted gene correction or replacement. In some embodiments, inactivation of a gene selected from the group consisting of TCRα, TCRβ, CD52, GR, DCK, and immune checkpoint proteins, can be done at a precise genomic location targeted by a specific TALE-nuclease, wherein said specific TALE-nuclease catalyzes a cleavage and wherein the exogenous nucleic acid successively comprising at least a region of homology and a sequence to inactivate one targeted gene selected from the group consisting of TCRα, TCRβ, CD52, GR, DCK, immune checkpoint proteins which is integrated by homologous recombination. In some embodiments, several genes can be, successively or at the same time, inactivated by using several TALE-nucleases respectively and specifically targeting one defined gene and several specific polynucleotides for specific gene inactivation.

In some embodiments, the method comprises inactivation of one or more additional genes selected from the group consisting of TCRα, TCRβ, CD52, GR, DCK, and immune checkpoint proteins. In some embodiments, inactivation of a gene can be accomplished by introducing into the cells at least one rare-cutting endonuclease such that the rare-cutting endonuclease specifically catalyzes cleavage in a targeted sequence of the cell genome; and optionally, introducing into the cells an exogenous nucleic acid successively comprising a first region of homology to sequences upstream of the cleavage, a sequence to be inserted in the genome of the cell, and a second region of homology to sequences downstream of the cleavage; wherein the introduced exogenous nucleic acid inactivates a gene and integrates at least one exogenous polynucleotide sequence encoding at least one recombinant protein of interest. In some embodiments, the exogenous polynucleotide sequence is integrated within a gene encoding a protein selected from the group consisting of TCRα, TCRβ, CD52, GR, DCK, and immune checkpoint protein.

In another aspect, a step of genetically modifying cells can comprise: modifying T cells by inactivating at least one gene expressing a target for an immunosuppressive agent, and; expanding the cells, optionally in presence of the immunosuppressive agent. An immunosuppressive agent is an agent that suppresses immune function by one of several mechanisms of action. An immunosuppressive agent can diminish the extent and/or voracity of an immune response. Non-limiting examples of immunosuppressive agents include calcineurin inhibitors, targets of rapamycin, interleukin-2 α-chain blockers, inhibitors of inosine monophosphate dehydrogenase, inhibitors of dihydrofolic acid reductase, corticosteroids, and immunosuppressive antimetabolites. Some cytotoxic immunosuppressants act by inhibiting DNA synthesis. Others may act through activation of T cells or by inhibiting the activation of helper cells. The methods according to the invention allow conferring immunosuppressive resistance to T cells for immunotherapy by inactivating the target of the immunosuppressive agent in T cells. As non-limiting examples, targets for immunosuppressive agent can be a receptor for an immunosuppressive agent such as for example without limitation CD52, glucocorticoid receptor (GR), FKBP family gene members, and cyclophilin family gene members.

In some embodiments, the genetic modification of the method involves expression, in provided cells to engineer, of one rare-cutting endonuclease such that the rare-cutting endonuclease specifically catalyzes cleavage in one targeted gene, thereby inactivating the targeted gene. In some embodiments, a method of engineering cells comprises at least one of the following steps: providing a T cell, such as from a cell culture or from a blood sample; selecting a gene in the T cell expressing a target for an immunosuppressive agent; introducing into the T cell a rare-cutting endonuclease able to selectively inactivate by DNA cleavage, e.g. by double-strand break the gene encoding a target for the immunosuppressive agent, and expanding the cells, optionally in presence of the immunosuppressive agent.

In some embodiments, the method comprises: providing a T cell, such as from a cell culture or from a blood sample; selecting a gene in the T cell wherein the gene expresses a target for an immunosuppressive agent; transfecting the T cell with nucleic acid encoding a rare-cutting endonuclease able to selectively inactivate by DNA cleavage, for example by double-strand break the gene encoding a target for the immunosuppressive agent, and expressing the rare-cutting endonucleases into the T cells; and expanding the cells, optionally in presence of the immunosuppressive agent.

In some embodiments, the rare-cutting endonuclease specifically targets CD52 or GR. In some embodiments, the gene selected for inactivation encodes CD52, and the immunosuppressive treatment comprises a humanized antibody targeting CD52 antigen. In some embodiments, the gene selected for inactivation encodes GR, and the immunosuppressive treatment comprises a corticosteroid such as dexamethasone. In some embodiments, the gene selected for inactivation is a FKBP family gene member or a variant thereof and the immunosuppressive treatment comprises FK506, also known as Tacrolimus or fujimycin. In some embodiments, the FKBP family gene member is FKBP12 or a variant thereof. In some embodiments, gene selected for inactivation is a cyclophilin family gene member or a variant thereof and the immunosuppressive treatment comprises cyclosporine.

In some embodiments, the rare-cutting endonuclease can be, for example, a meganuclease, a zinc finger nuclease, or a TALE-nuclease (TALEN). In some embodiments, the rare-cutting endonuclease is a TALE-nuclease.

Also provided herein are methods of engineering T cells, suitable for immunotherapy, wherein the methods comprise: genetically modifying T cells by inactivating at least immune checkpoint protein. In some embodiments the immune checkpoint protein is, for example, PD-1 and/or CTLA-4. In some embodiments, methods of genetically modifying a cell comprises: modifying T cells by inactivating at least one immune checkpoint protein; and expanding the cells. Immune checkpoint proteins include, but are not limited to Programmed Death 1 (PD-1, also known as PDCD1 or CD279, accession number: NM 005018), Cytotoxic T-Lymphocyte Antigen 4 (CTLA-4, also known as CD152, GenBank accession number AF414120.1), LAG3 (also known as CD223, accession number: NM 002286.5), Tim3 (also known as HAVCR2, GenBank accession number: JX049979.1), BTLA (also known as CD272, accession number: NM 181780.3), BY55 (also known as CD160, GenBank accession number: CR541888.1), TIGIT (also known as VSTM3, accession number: NM 173799), B7H5 (also known as C10orf54, homolog of mouse vista gene, accession number: NM 022153.1), LAIR1 (also known as CD305, GenBank accession number: CR542051.1), SIGLEC10 (GeneBank accession number: AY358337.1), 2B4 (also known as CD244, accession number: NM 001166664.1), which directly inhibit immune cells. For example, CTLA-4 is a cell-surface protein expressed on certain CD4 and CD8 T cells; when engaged by its ligands (B7-1 and B7-2) on antigen presenting cells, T cell activation and effector function are inhibited.

In some embodiments, said method to engineer cells comprises at least one of the following steps: providing a T cell, such as from a cell culture or from a blood sample; introducing into the T cell a rare-cutting endonuclease able to selectively inactivate by DNA cleavage, for example by double-strand break one gene encoding a immune checkpoint protein; and expanding the cells. In some embodiments, the method comprises: providing a T cell, such as from a cell culture or from a blood sample; transfecting said T cell with nucleic acid encoding a rare-cutting endonuclease able to selectively inactivate by DNA cleavage, for example by double-strand break a gene encoding a immune checkpoint protein; expressing the rare-cutting endonucleases into the T cells; expanding the cells. In some embodiments, the rare-cutting endonuclease specifically targets a gene selected from the group consisting of: PD-1, CTLA-4, LAG3, Tim3, BTLA, BY55, TIGIT, B7H5, LAIR1, SIGLEC10, 2B4, TCRα, and TCRβ. In some embodiments, the rare-cutting endonuclease can be a meganuclease, a zinc finger nuclease or a TALE-nuclease. In some embodiments, the rare-cutting endonuclease is a TALE-nuclease.

In some embodiments, the present invention can be particularly suitable for allogeneic immunotherapy. In such embodiments, cells may be modified by a method comprising: inactivating at least one gene encoding a component of the T cell receptor (TCR) in T cells; and expanding the T cells. In some embodiments, the genetic modification of the method relies on the expression, in provided cells to engineer, of one rare-cutting endonuclease such that the rare-cutting endonuclease specifically catalyzes cleavage in one targeted gene thereby inactivating the targeted gene. In some embodiments, said method to engineer cells comprises at least one of the following steps: providing a T cell, such as from a cell culture or from a blood sample; introducing into the T cell a rare-cutting endonuclease able to selectively inactivate by DNA cleavage, for example by double-strand break at least one gene encoding a component of the T cell receptor (TCR), and expanding the cells.

In some embodiments, the method comprises: providing a T cell, such as from a cell culture or from a blood sample; transfecting said T cell with nucleic acid encoding a rare-cutting endonuclease able to selectively inactivate by DNA cleavage, for example by double-strand break at least one gene encoding a component of the T cell receptor (TCR); expressing the rare-cutting endonucleases into the T cells; sorting the transformed T cells, which do not express TCR on their cell surface; and expanding the cells.

In some embodiments, the rare-cutting endonuclease can be a meganuclease, a zinc finger nuclease or a TALE-nuclease. In some embodiments, the rare-cutting endonuclease is a TALE-nuclease. In some embodiments, the TALE-nucleases recognize and cleave a sequence encoding TCRa or TCRβ. In some embodiments, a TALE-nuclease comprises a polypeptide sequence selected from the amino acid sequence shown in SEQ ID NOs: 71, 72, 73, 74, 75, 76, 77, and 78.

### TALE-Nuclease Polypeptide Sequences:

Repeat TRAC_T01-L
(SEQ ID NO: 71)
LTPQQVVAIASNGGGKQALETVQRLLPVLCQAHGLTPQQVVAIASNNGGKQALET
VQRLLPVLCQAHGLTPQQVVAIASNGGGKQALETVQRLLPVLCQAHGLTPEQVVA
IASHDGGKQALETVQRLLPVLCQAHGLTPEQVVAIASHDGGKQALETVQRLLPVLC
QAHGLTPEQVVAIASHDGGKQALETVQRLLPVLCQAHGLTPEQVVAIASNIGGKQA
LETVQALLPVLCQAHGLTPEQVVAIASHDGGKQALETVQRLLPVLCQAHGLTPEQ
VVAIASNIGGKQALETVQALLPVLCQAHGLTPQQVVAIASNNGGKQALETVQRLLP
VLCQAHGLTPEQVVAIASNIGGKQALETVQALLPVLCQAHGLTPQQVVAIASNGGG
KQALETVQRLLPVLCQAHGLTPEQVVAIASNIGGKQALETVQALLPVLCQAHGLTP
QQVVAIASNGGGKQALETVQRLLPVLCQAHGLTPEQVVAIASHDGGKQALETVQR
LLPVLCQAHGLTPQQVVAIASNGGGRPALE.
Repeat TRAC_T01-R
(SEQ ID NO: 72)
LTPEQVVAIASHDGGKQALETVQRLLPVLCQAHGLTPQQVVAIASNGGGKQALET
VQRLLPVLCQAHGLTPEQVVAIASHDGGKQALETVQRLLPVLCQAHGLTPEQVVAI
ASNIGGKQALETVQALLPVLCQAHGLTPQQVVAIASNNGGKQALETVQRLLPVLC
QAHGLTPEQVVAIASHDGGKQALETVQRLLPVLCQAHGLTPQQVVAIASNGGGKQ
ALETVQRLLPVLCQAHGLTPQQVVAIASNNGGKQALETVQRLLPVLCQAHGLTPQ
QVVAIASNNGGKQALETVQRLLPVLCQAHGLTPQQVVAIASNGGGKQALETVQRL
LPVLCQAHGLTPEQVVAIASNIGGKQALETVQALLPVLCQAHGLTPEQVVAIASHD
GGKQALETVQRLLPVLCQAHGLTPEQVVAIASNIGGKQALETVQALLPVLCQAHG
LTPEQVVAIASHDGGKQALETVQRLLPVLCQAHGLTPQQVVAIASNNGGKQALET
VQRLLPVLCQAHGLTPQQVVAIASNGGGRPALE.
Repeat TRBC_T01-L
(SEQ ID NO: 73)
LTPQQVVAIASNNGGKQALETVQRLLPVLCQAHGLTPQQVVAIASNGGGKQALET
VQRLLPVLCQAHGLTPQQVVAIASNNGGKQALETVQRLLPVLCQAHGLTPQQVVA
IASNGGGKQALETVQRLLPVLCQAHGLTPQQVVAIASNGGGKQALETVQRLLPVL
CQAHGLTPQQVVAIASNGGGKQALETVQRLLPVLCQAHGLTPQQVVAIASNNGGK
QALETVQRLLPVLCQAHGLTPEQVVAIASNIGGKQALETVQALLPVLCQAHGLTPQ
QVVAIASNNGGKQALETVQRLLPVLCQAHGLTPEQVVAIASHDGGKQALETVQRL
LPVLCQAHGLTPEQVVAIASHDGGKQALETVQRLLPVLCQAHGLTPEQVVAIASNI
GGKQALETVQALLPVLCQAHGLTPQQVVAIASNGGGKQALETVQRLLPVLCQAHG
LTPEQVVAIASHDGGKQALETVQRLLPVLCQAHGLTPEQVVAIASNIGGKQALETV
QALLPVLCQAHGLTPQQVVAIASNGGGRPALE.
Repeat TRBC_T01-R
(SEQ ID NO: 74)
NPQRSTVWYLTPQQVVAIASNNGGKQALETVQRLLPVLCQAHGLTPQQVVAIASN
GGGKQALETVQRLLPVLCQAHGLTPQQVVAIASNNGGKQALETVQRLLPVLCQAH
GLTPQQVVAIASNNGGKQALETVQRLLPVLCQAHGLTPEQVVAIASHDGGKQALE
TVQRLLPVLCQAHGLTPEQVVAIASHDGGKQALETVQRLLPVLCQAHGLTPQQVV
AIASNGGGKQALETVQRLLPVLCQAHGLTPQQVVAIASNGGGKQALETVQRLLPV
LCQAHGLTPQQVVAIASNGGGKQALETVQRLLPVLCQAHGLTPQQVVAIASNGGG
KQALETVQRLLPVLCQAHGLTPQQVVAIASNNGGKQALETVQRLLPVLCQAHGLT
PQQVVAIASNNGGKQALETVQRLLPVLCQAHGLTPQQVVAIASNNGGKQALETVQ
RLLPVLCQAHGLTPQQVVAIASNGGGKQALETVQRLLPVLCQAHGLTPQQVVAIAS
NNGGKQALETVQRLLPVLCQAHGLTPQQVVAIASNGGGRPALE.
Repeat TRBC_T02-L
(SEQ ID NO: 75)
LTPEQVVAIASHDGGKQALETVQRLLPVLCQAHGLTPEQVVAIASHDGGKQALET
VQRLLPVLCQAHGLTPEQVVAIASHDGGKQALETVQRLLPVLCQAHGLTPEQVVAI
ASNIGGKQALETVQALLPVLCQAHGLTPEQVVAIASHDGGKQALETVQRLLPVLCQ
AHGLTPEQVVAIASHDGGKQALETVQRLLPVLCQAHGLTPEQVVAIASHDGGKQA
LETVQRLLPVLCQAHGLTPQQVVAIASNNGGKQALETVQRLLPVLCQAHGLTPEQ
VVAIASNIGGKQALETVQALLPVLCQAHGLTPQQVVAIASNNGGKQALETVQRLLP
VLCQAHGLTPQQVVAIASNNGGKQALETVQRLLPVLCQAHGLTPQQVVAIASNGG
GKQALETVQRLLPVLCQAHGLTPEQVVAIASHDGGKQALETVQRLLPVLCQAHGL
TPQQVVAIASNNGGKQALETVQRLLPVLCQAHGLTPEQVVAIASHDGGKQALETV
QRLLPVLCQAHGLTPQQVVAIASNGGGRPALE.
Repeat TRBC_T02-R
(SEQ ID NO: 76)
LTPQQVVAIASNNGGKQALETVQRLLPVLCQAHGLTPQQVVAIASNNGGKQALET
VQRLLPVLCQAHGLTPQQVVAIASNNGGKQALETVQRLLPVLCQAHGLTPEQVVA
IASNIGGKQALETVQALLPVLCQAHGLTPQQVVAIASNNGGKQALETVQRLLPVLC
QAHGLTPEQVVAIASNIGGKQALETVQALLPVLCQAHGLTPQQVVAIASNGGGKQ
ALETVQRLLPVLCQAHGLTPEQVVAIASHDGGKQALETVQRLLPVLCQAHGLTPQ
QVVAIASNGGGKQALETVQRLLPVLCQAHGLTPEQVVAIASHDGGKQALETVQRL
LPVLCQAHGLTPQQVVAIASNGGGKQALETVQRLLPVLCQAHGLTPQQVVAIASN
NGGKQALETVQRLLPVLCQAHGLTPEQVVAIASHDGGKQALETVQRLLPVLCQAH
GLTPQQVVAIASNGGGKQALETVQRLLPVLCQAHGLTPQQVVAIASNGGGKQALE
TVQRLLPVLCQAHGLTPQQVVAIASNGGGRPALE.
Repeat CD52_T02-L
(SEQ ID NO: 77)
LTPQQVVAIASNGGGKQALETVQRLLPVLCQAHGLTPEQVVAIASHDGGKQALET
VQRLLPVLCQAHGLTPEQVVAIASHDGGKQALETVQRLLPVLCQAHGLTPQQVVA
IASNGGGKQALETVQRLLPVLCQAHGLTPEQVVAIASHDGGKQALETVQRLLPVLC
QAHGLTPEQVVAIASHDGGKQALETVQRLLPVLCQAHGLTPQQVVAIASNGGGKQ
ALETVQRLLPVLCQAHGLTPEQVVAIASNIGGKQALETVQALLPVLCQAHGLTPEQ
VVAIASHDGGKQALETVQRLLPVLCQAHGLTPQQVVAIASNGGGKQALETVQRLL
PVLCQAHGLTPEQVVAIASHDGGKQALETVQRLLPVLCQAHGLTPEQVVAIASNIG
GKQALETVQALLPVLCQAHGLTPEQVVAIASHDGGKQALETVQRLLPVLCQAHGL
TPEQVVAIASHDGGKQALETVQRLLPVLCQAHGLTPEQVVAIASNIGGKQALETVQ
ALLPVLCQAHGLTPQQVVAIASNGGGRPALE.
Repeat CD52_T02-R
(SEQ ID NO: 78)
LTPQQVVAIASNGGGKQALETVQRLLPVLCQAHGLTPQQVVAIASNNGGKQALET
VQRLLPVLCQAHGLTPEQVVAIASHDGGKQALETVQRLLPVLCQAHGLTPQQVVA
IASNGGGKQALETVQRLLPVLCQAHGLTPEQVVAIASHDGGKQALETVQRLLPVLC
QAHGLTPQQVVAIASNGGGKQALETVQRLLPVLCQAHGLTPQQVVAIASNGGGKQ
ALETVQRLLPVLCQAHGLTPEQVVAIASNIGGKQALETVQALLPVLCQAHGLTPEQ
VVAIASHDGGKQALETVQRLLPVLCQAHGLTPEQVVAIASHDGGKQALETVQRLL
PVLCQAHGLTPQQVVAIASNGGGKQALETVQRLLPVLCQAHGLTPQQVVAIASNN
GGKQALETVQRLLPVLCQAHGLTPQQVVAIASNGGGKQALETVQRLLPVLCQAHG
LTPEQVVAIASNIGGKQALETVQALLPVLCQAHGLTPEQVVAIASHDGGKQALETV
QRLLPVLCQAHGLTPQQVVAIASNGGGRPALE.

In another aspect, another step of genetically modifying a cell can be a method of expanding TCRα deficient T cells comprising introducing into the T cell pTα (also known as preTCRα) or a functional variant thereof and expanding the cells, optionally through stimulation of the CD3 complex. In some embodiments, the method comprises: a) transfecting the cells with nucleic acid encoding at least a fragment of pTα to support CD3 surface expression; b) expressing said pTα into the cells; and c) expanding the cells, optionally through stimulation of the CD3 complex.

Also provided are methods of preparing T cells for immunotherapy comprising steps of the method for expansion for T cell. In some embodiments, the pTα polynucleotide sequence can be introduced randomly or by homologous recombination. In some embodiments, the insertion can be associated with the inactivation of the TCRα gene.

Different functional variants of pTα can be used. A “functional variant” of the peptide refers to a molecule substantially similar to either the entire peptide or a fragment thereof. A “fragment” of the pTα or functional variant thereof refers to any subset of the molecule, that is, a shorter peptide than the full-length pTα. In some embodiments, pTα or functional variants can be, for example, full-length pTα or a C-terminal truncated pTα version. C-terminal truncated pTα lacks in C-terminal end one or more residues. As non limiting examples, C-terminal truncated pTα version lacks 18, 48, 62, 78, 92, 110 or 114 residues from the C-terminus of the protein. Amino acid sequence variants of the peptide can be prepared by mutations in the DNA which encodes the peptide. Such functional variants include, for example, deletions from, or insertions or substitutions of, residues within the amino acid sequence. Any combination of deletion, insertion, and substitution may also be made to arrive at the final construct, provided that the final construct possesses the desired activity, in particular the restoration of a functional CD3 complex. In an exemplary embodiment, at least one mutation is introduced in the different pTα versions as described herein to affect dimerization. As non limiting example, mutated residue can be at least W46R, D22A, K24A, R102A or R117A of the human pTα protein or aligned positions using CLUSTALW method on pTα family or homologue member. For example pTα or variant thereof as described herein comprise the mutated residue W46R or the mutated residues D22A, K24A, R102A and R117A. In some embodiments, said pTα or variants are also fused to a signal-transducing domain such as CD28, OX40, ICOS, CD27, CD137 (4-1BB) and CD8 as non limiting examples. The extracellular domain of pTα or variants as described herein can be fused to a fragment of the TCRα protein, particularly the transmembrane and intracellular domain of TCRα. pTα variants can also be fused to the intracellular domain of TCRα.

In some embodiments, pTα versions can be fused to an extracellular ligand-binding domain. In some embodiments, pTα or functional variant thereof is fused to a single chain antibody fragment (scFv) comprising the light and the heavy variable fragment of a target antigen specific monoclonal antibody joined by a flexible linker.

The term “TCRα deficient T cell” refers to an isolated T cell that lacks expression of a functional TCRα chain. Such cells may be prepared by various means, as non-limiting examples, by engineering a T cell such that it does not express any functional TCRα on its cell surface or by engineering a T cell such that it produces very little functional TCRα chain on its surface or by engineering a T cell to express mutated or truncated form of TCRα chain. TCRα deficient cells can no longer be expanded through CD3 complex. Thus, to overcome this problem and to allow proliferation of TCRα deficient cells, pTα or functional variant thereof is introduced into the cells, thus restoring a functional CD3 complex. In some embodiments, the method further comprises introducing into said T cells rare-cutting endonucleases able to selectively inactivate by DNA cleavage one gene encoding one component of the T cell receptor (TCR). In some embodiments, the rare-cutting endonuclease is a TALE-nuclease.

In some embodiments, polynucleotides encoding polypeptides according to the present invention can be mRNA which is introduced directly into the cells, for example by electroporation. In some embodiments, cytoPulse technology can be used to transiently permeabilize living cells for delivery of material into the cells. Parameters can be modified in order to determine conditions for high transfection efficiency with minimal mortality.

Also provided herein are methods of transfecting T cell. In some embodiments, the method comprises: contacting a T cell with RNA and applying to T cell an agile pulse sequence consisting of: (a) an electrical pulse with a voltage range from about 2250 to 3000 V per centimeter; (b) a pulse width of 0.1 ms; (c) a pulse interval of about 0.2 to 10 ms between the electrical pulses of step (a) and (b); (d) an electrical pulse with a voltage range from about 2250 to 3000 V with a pulse width of about 100 ms and a pulse interval of about 100 ms between the electrical pulse of step (b) and the first electrical pulse of step (c); and (e) four electrical pulses with a voltage of about 325 V with a pulse width of about 0.2 ms and a pulse interval of 2 ms between each of 4 electrical pulses. In some embodiments, a method of transfecting T cell comprising contacting said T cell with RNA and applying to T cell an agile pulse sequence comprising: (a) an electrical pulse with a voltage of about 2250, 2300, 2350, 2400, 2450, 2500, 2550, 2400, 2450, 2500, 2600, 2700, 2800, 2900 or 3000V per centimeter; (b) a pulse width of 0.1 ms; (c) and a pulse interval of about 0.2, 0.5, 1, 2, 3, 4, 5, 6, 7, 8, 9 or 10 ms between the electrical pulses of step (a) and (b); (d) one electrical pulse with a voltage range from about 2250, of 2250, 2300, 2350, 2400, 2450, 2500, 2550, 2400, 2450, 2500, 2600, 2700, 2800, 2900 or 3000V with a pulse width of 100 ms and a pulse interval of 100 ms between the electrical pulse of step (b) and the first electrical pulse of step (c); and (e) 4 electrical pulses with a voltage of about 325 V with a pulse width of about 0.2 ms and a pulse interval of about 2 ms between each of 4 electrical pulses. Any values included in the value range described above are disclosed in the present application. Electroporation medium can be any suitable medium known in the art. In some embodiments, the electroporation medium has conductivity in a range spanning about 0.01 to about 1.0 milliSiemens.

In some embodiments, as non limiting examples, an RNA encodes a rare-cutting endonuclease, one monomer of the rare-cutting endonuclease such as half-TALE-nuclease, a CAR, at least one component of the multi-chain chimeric antigen receptor, a pTα or functional variant thereof, an exogenous nucleic acid, and/or one additional catalytic domain.

### Engineered Immune Cells

The invention also provides engineered immune cells comprising any of the polynucleotides that encode the recombinant antigen receptors e.g. CARs described herein. In some embodiments, such an encoding polynucleotide can be introduced into an immune cell as a transgene via a plasmid vector. In some embodiments, the plasmid vector can also contain, for example, a selection marker which provides for identification and/or selection of cells which received the vector.

Recombinant antigen receptor e.g. CAR polypeptides may be synthesized in situ in the cell after introduction of polynucleotides encoding the polypeptides into the cell. Alternatively, the polypeptides may be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides may be introduced into a cell by any suitable means such as for example, recombinant viral vectors (e.g. retroviruses, adenoviruses), liposomes, and the like. Transient transformation methods include, for example without limitation, microinjection, electroporation or particle bombardment. Polynucleotides may be included in vectors, such as for example plasmid vectors or viral vectors.

In some embodiments, the engineered immune cells disclosed herein further comprise one or more polynucleotides that express one or more polypeptides that enhance the efficacy, activity, proliferation and/or persistence of the engineered immune cell, In certain embodiments, the engineered immune cells further express one or more chimeric cytokine receptors that provide the signal 3 of TCR signaling. In some embodiments, the chimeric cytokine receptors are inducible or constitutively active. Exemplary chimeric cytokine receptors are described in WO2020/180694, WO2020/180664, and WO2021/041806, all of which are incorporated herein by reference.

In some embodiments, the engineered immune cells disclosed herein further comprise one or more polynucleotides that express one or more polypeptides that reduce reactivity of the allogeneic engineered immune cells in a recipient. In certain embodiments, the engineered immune cells further express one or more autologous/allogeneic immune defense receptors. Exemplary autologous/allogeneic immune defense receptors are described in WO2019/210081, which is herein incorporated by reference.

Also provided herein are isolated cells and cell lines obtained by the herein-described methods of engineering cells provided herein. In some embodiments, an isolated cell comprises at least one recombinant antigen receptor e.g. CAR as described herein. In some embodiments, an isolated cell comprises a population of different recombinant antigen receptors e.g. CARs, each species of which comprises different extracellular ligand-binding domains.

Also provided herein are isolated immune cells obtained according to any one of the methods described herein. Any immune cell capable of expressing heterologous DNAs can be used for the purpose of expressing the polypeptide of interest. In some embodiments, the immune cell used for expressing any one of the recombinant antigen receptors e.g. CARs described herein is a T cell. In some embodiments, an immune cell used for expressing CARs can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human stem cells are CD34+ cells.

In some embodiments, the engineered immune cells expressing at their cell surface membrane a recombinant antigen receptor e.g. CAR of the invention comprise a percentage of stem cell memory and central memory cells greater than 10%, 20%, 30%, 40%, 50%, or 60%. In some embodiments, the engineered immune cells expressing at their cell surface membrane a recombinant antigen receptor e.g. CAR of the invention comprise a percentage of stem cell memory and central memory cells of about 10% to about 60%, about 10% to about 50%, about 10% to about 40%, about 15% to about 50%, about 15% to about 40%, about 20% to about 60%, or about 20% to about 70%.

The immune cell used for expressing any one of the recombinant antigen receptors e.g. CARs described herein can also be a dendritic cell, killer dendritic cell, a mast cell, a NK-cell, a B-cell or a T cell selected from the group consisting of inflammatory T-lymphocytes, cytotoxic T-lymphocytes, regulatory T-lymphocytes or helper T-lymphocytes. In some embodiments, the cell can be derived from the group consisting of CD4+ T-lymphocytes and CD8+ T-lymphocytes.

In one embodiment, the immune cell is an inflammatory T-lymphocyte that expresses any one of the CARs described herein. In one embodiment, the immune cell is a cytotoxic T-lymphocyte that expresses any one of the CARs described herein. In one embodiment, the immune cell is a regulatory T-lymphocyte that expresses any one of the CARs described herein. In one embodiment, the immune cell is a helper T-lymphocyte that expresses any one of the CARs described herein.

Also provided herein are cell lines obtained from a transformed T cell according to any of the herein-described methods. Also provided herein are modified cells resistant to an immunosuppressive treatment. In some embodiments, an isolated cell according to the invention comprises a polynucleotide encoding a CAR.

The immune cells of the invention can be activated and expanded, either prior to or after genetic modification of the T cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo. Generally, the T cells of the invention can be expanded, for example, by contact with an agent that stimulates a CD3 TCR complex and a co-stimulatory molecule on the surface of the T cells to create an activation signal for the T cell. For example, chemicals such as calcium ionophore A23187, phorbol 12-myristate 13-acetate (PMA), or mitogenic lectins like phytohemagglutinin (PHA) can be used to create an activation signal for the T cell.

In some embodiments, T cell populations may be stimulated in vitro by contact with, for example, an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-7, IL-4, IL-7, GM-CSF, IL-10, IL-2, IL-15, TGFp, and TNF, or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics

In some embodiments, the cells of the invention can be expanded by co-culturing with tissue or cells. The cells can also be expanded in vivo, for example in the subject's blood after administrating the cell into the subject.

In some embodiments, an isolated cell according to the present invention comprises one inactivated gene selected from the group consisting of CD52, GR, PD-1, CTLA-4, LAG3, Tim3, BTLA, BY55, TIGIT, B7H5, LAIR1, SIGLEC10, 2B4, HLA, TCRα and TCRP and/or expresses a CAR, a multi-chain CAR and/or a pTα transgene. In some embodiments, an isolated cell comprises polynucleotides encoding polypeptides comprising a multi-chain CAR. In some embodiments, the isolated cell according to the present invention comprises two inactivated genes selected from the group consisting of: CD52 and GR, CD52 and TCRα, CDR52 and TCRβ, GR and TCRα, GR and TCRβ, TCRα and TCRβ, PD-1 and TCRα, PD-1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ and/or expresses a CAR, a multi-chain CAR and a pTα transgene.

In some embodiments, TCR is rendered not functional in the cells according to the invention by inactivating TCRα gene and/or TCRβ gene(s). In some embodiments, a method to obtain modified cells derived from an individual is provided, wherein the cells can proliferate independently of the major histocompatibility complex (MHC) signaling pathway. Modified cells, which can proliferate independently of the MHC signaling pathway, susceptible to be obtained by this method are encompassed in the scope of the present invention. Modified cells disclosed herein can be used in for treating individuals in need thereof against Host versus Graft (HvG) rejection and Graft versus Host Disease (GvHD); therefore in the scope of the present invention is a method of treating individuals in need thereof against Host versus Graft (HvG) rejection and Graft versus Host Disease (GvHD) comprising treating said individual by administering to said individual an effective amount of modified cells comprising inactivated TCRα and/or TCRβ genes.

In some embodiments, the immune cells are engineered to be resistant to one or more chemotherapy drugs. The chemotherapy drug can be, for example, a purine nucleotide analogue (PNA), thus making the immune cell suitable for cancer treatment combining adoptive immunotherapy and chemotherapy. Exemplary PNAs include, for example, clofarabine, fludarabine, and cytarabine, alone or in combination. PNAs are metabolized by deoxycytidine kinase (dCK) into mono-, di-, and tri-phosphate PNA. Their tri-phosphate forms compete with ATP for DNA synthesis, act as pro-apoptotic agents, and are potent inhibitors of ribonucleotide reductase (RNR), which is involved in trinucleotide production. Provided herein are target-specific CAR-T cells comprising an inactivated dCK gene. In some embodiments, the dCK knockout cells are made by transfection of T cells using polynucleotides encoding specific TAL-nulcease directed against dCK genes by, for example, electroporation of mRNA. The dCK knockout target-specific CAR-T cells are resistant to PNAs, including for example clorofarabine and/or fludarabine, and maintain T cell cytotoxic activity toward FLT3-expressing cells.

In some embodiments, isolated cells or cell lines of the invention can comprise a pTα or a functional variant thereof. In some embodiments, an isolated cell or cell line can be further genetically modified by inactivating the TCRα gene.

In some embodiments, the CAR-T cell comprises a polynucleotide encoding a suicide polypeptide, such as for example RQR8. See, e.g., WO2013153391A, which is hereby incorporated by reference in its entirety. In CAR-T cells comprising the polynucleotide, the suicide polypeptide is expressed at the surface of a CAR-T cell. In some embodiments, the suicide polypeptide comprises the amino acid sequence shown in SEQ ID NO: 79.

(SEQ ID NO: 79)
CPYSNPSLCSGGGGSELPTQGTFSNVSTNVSPAKPTTTACPYSNPSLCS
GGGGSPAPRPPTPAPTIASQPLSLRPEACRPAAGGAVHTRGLDFACDIY
IWAPLAGTCGVLLLSLVITLYCNHRNRRRVCKCPRPVV.

The suicide polypeptide may also comprise a signal peptide at the amino terminus. In some embodiments, the suicide polypeptide comprises the amino acid sequence shown in SEQ ID NO: 80.

(SEQ ID NO: 80)
MGTSLLCWMALCLLGADHADACPYSNPSLCSGGGGSELPTQGTFSNVST
NVSPAKPTTTACPYSNPSLCSGGGGSPAPRPPTPAPTIASQPLSLRPEA
CRPAAGGAVHTRGLDFACDIYIWAPLAGTCGVLLLSLVITLYCNHRNRR
RVCKCPRPVV.

When the suicide polypeptide is expressed at the surface of a CAR-T cell, binding of rituximab to the rituximab epitopes of the polypeptide causes lysis of the cell. More than one molecule of rituximab may bind per polypeptide expressed at the cell surface. Each rituximab epitope of the polypeptide may bind a separate molecule of rituximab. Deletion of target-specific CAR-T cells may occur in vivo, for example by administering rituximab to a subject. The decision to delete the transferred cells may arise from undesirable effects being detected in the subject which are attributable to the transferred cells, such as for example, when unacceptable levels of toxicity are detected.

In some embodiments, upon administration to a patient, engineered immune cells expressing at their cell surface any one of the recombinant antigen receptors e.g. CARs described herein may reduce, kill or lyse endogenous cells of the patient that express the target antigen (e.g. DLL3, BCMA, EGFRvIII, Flt-3, WT-1, CD20, CD23, CD30, CD38, CD70, CD33, CD133, LeY, NKG2D, CS1, CD44v6, ROR1, CD19, Claudin-18.2 (Claudin-18A2, or Claudin18 isoform 2), DLL3 (Delta-like protein 3, Drosophila Delta homolog 3, Delta3), Muc16, Muc17 (Mucin17, Muc3), FAP alpha (Fibroblast Activation Protein alpha), Ly6G6D (Lymphocyte antigen 6 complex locus protein G6d, c6orf23, G6D, MEGT1, NG25), and/or RNF43 (E3 ubiquitin-protein ligase RNF43, RING finger protein 43)) of the recombinant antigen receptor e.g. CAR. In one embodiment, a percentage reduction or lysis of such endogenous cells or cells of a cell line expressing the target antigen by engineered immune cells expressing any one of the recombinant antigen receptors e.g. CARs described herein is at least about or greater than 10%, 15%, 20%, 25%, 30%, 35%, 40%, 45%, 50%, 55%, 60%, 65%, 70%, 75%, 80%, 85%, 90%, or 95%. In one embodiment, a percentage reduction or lysis of such target antigen-expressing endogenous cells or cells of a cell line expressing the target antigen by engineered immune cells expressing any one of the target-specific CARs, such as DLL3 CAR described herein, is about 5% to about 95%, about 10% to about 95%, about 10% to about 90%, about 10% to about 80%, about 10% to about 70%, about 10% to about 60%, about 10% to about 50%, about 10% to about 40%, about 20% to about 90%, about 20% to about 80%, about 20% to about 70%, about 20% to about 60%, about 20% to about 50%, about 25% to about 75%, or about 25% to about 60%.

Method for Sorting Recombinant Antigen Receptor e.g. CAR-Positive Immune Cells

In one aspect, provided are methods for in vitro sorting of a population of immune cells, wherein a subset of the population of immune cells comprises engineered immune cells expressing any one of the recombinant antigen receptors e.g. CARs comprising epitopes specific for monoclonal antibodies described herein. The method comprises contacting the population of immune cells with a monoclonal antibody specific for the epitopes and selecting the immune cells that bind to the monoclonal antibody to obtain a population of cells enriched in engineered immune cells expressing the recombinant antigen receptor e.g. CAR.

In some embodiments, said monoclonal antibody specific for said epitope is optionally conjugated to a fluorophore. In this embodiment, the step of selecting the cells that bind to the monoclonal antibody can be done by Fluorescence Activated Cell Sorting (FACS). In some embodiments, said monoclonal antibody specific for said epitope is optionally conjugated to a magnetic particle. In this embodiment, the step of selecting the cells that bind to the monoclonal antibody can be done by Magnetic Activated Cell Sorting (MACS).

In some embodiments, the population of recombinant antigen receptor-expressing e.g. CAR-expressing immune cells obtained when using the method for in vitro sorting of immune cells described herein, comprises at least 60%, 65%, 70%, 75%, 80%, 85%, 90%, or 95% of the recombinant antigen receptor-expressing e.g. CAR-expressing immune cells. In some embodiments, the population of expressing immune cells obtained when using the method for in vitro sorting of CAR-expressing immune cells described herein, comprises at least 85% of recombinant antigen receptor-expressing e.g. CAR-expressing immune cells.

In some embodiments, the mAbs used in the in vitro sorting method are previously bound onto a support such as a column or on beads such as routinely realized by the skilled in the art. In some embodiments, immune cells expressing CARs are T-cells.

According to the invention, cells to be administered to the recipient may be enriched in vitro from the source population. Methods of expanding source populations are well known in the art, and may include selecting cells that express an antigen such as CD34 antigen, using combinations of density centrifugation, immuno-magnetic bead purification, affinity chromatography, and fluorescent activated cell sorting, known to those skilled in the art.

Flow cytometry is widely used in the art and is a method well known to one of ordinary skill to sort and quantify specific cell types within a population of cells. In general, flow cytometry is a method for quantitating components or structural features of cells primarily by optical means. Since different cell types can be distinguished by quantitating structural features, flow cytometry and cell sorting can be used to count and sort cells of different phenotypes in a mixture.

A flow cytometric analysis involves two basic steps: 1) labeling selected cell types with one or more labeled markers, and 2) determining the number of labeled cells relative to the total number of cells in the population.

The primary method of labeling cell types is by binding labeled antibodies to markers expressed by the specific cell type. The antibodies are either directly labeled with a fluorescent compound or indirectly labeled using, for example, a fluorescent-labeled second antibody which recognizes the first antibody.

In some embodiments, the method used for sorting immune cells expressing a CAR is the Magnetic-Activated Cell Sorting (MACS).

Magnetic-activated cell sorting (MACS) is a method for separation of various cell populations depending on their surface antigens (CD molecules) by using superparamagnetic nanoparticles and columns. It takes a few simple steps to get pure cell populations. Cells in a single-cell suspension are magnetically labeled with microbeads. The sample is applied to a column composed of ferromagnetic spheres, which are covered with a cell-friendly coating allowing fast and gentle separation of cells. The unlabeled cells pass through while the magnetically labeled cells are retained within the column. The flow-through can be collected as the unlabeled cell fraction. After a short washing step, the column is removed from the separator, and the magnetically labeled cells are eluted from the column.

In some embodiments, the mAb used in the method for sorting immune cells expressing the CAR is chosen from alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and/or ustekinumab. In some embodiments, said mAb is rituximab. In another embodiment, said mAb is QBEND-10.

### Therapeutic Applications

Isolated cells obtained by the methods described herein, or cell lines derived from such isolated cells, expressing recombinant antigen receptors of the invention e.g. CARs of the invention can be used as a medicament. In some embodiments, such a medicament can be used for treating a disease or a condition associated with the target antigen of the recombinant antigen receptor e.g CAR of the invention. Because the target specificity of the recombinant antigen receptors e.g. CARs of the present invention can be engineered toward the target of choice, conditions treatable with the recombinant antigen receptors e.g. CARs of the invention are diverse. These include but are not limited to various forms of cancer, such as, for example, cancers associated with the expression of DLL3.

In some embodiments, an isolated cell according to the invention, or cell line derived from the isolated cells, or an antibody can be used in the manufacture of a medicament for treatment of a cancer in a subject in need thereof.

In some embodiments, immune cells containing a recombinant antigen receptor, e.g., a CAR, of the disclosure can be used to treat such malignancies as small cell lung cancer, melanoma, low grade gliomas, glioma, glioblastoma, medullary thyroid cancer, carcinoids, dispersed neuroendocrine tumors in the pancreas, bladder and prostate, testicular cancer, lymphoma, leukemia, Renal Cell Carcinoma (RCC), Non-Hodgkin's Lymphoma, Hodgkin's Disease (HD), Waldenstrom's macroglobulinemia, Acute Myeloid Leukemia, Multiple Myeloma, diffuse large-cell lymphoma, follicular lymphoma, and lung adenocarcinomas with neuroendocrine features. In exemplary embodiments, the CAR-containing immune cells, e.g., the anti-DLL3 CAR-T cells of the disclosure, are used to treat small cell lung cancer.

Also provided herein are methods for treating subjects. In some embodiments, the method comprises providing an immune cell of the invention to a subject in need thereof. In some embodiments, the method comprises a step of administering transformed immune cells of the invention to a subject in need thereof.

In some embodiments, T cells of the invention can undergo robust in vivo T cell expansion and can persist for an extended amount of time.

Methods of treatment of the invention can be ameliorating, curative or prophylactic. The method of the invention may be either part of an autologous immunotherapy or part of an allogeneic immunotherapy treatment. The invention is particularly suitable for allogeneic immunotherapy. T cells from donors can be transformed into non-alloreactive cells using standard protocols and reproduced as needed, thereby producing CAR-T cells which may be administered to one or several subjects. Such CAR-T cell therapy can be made available as an “off the shelf” therapeutic product.

Cells that can be used with the disclosed methods are described in the previous section. Treatment can be used to treat subjects diagnosed with, for example, cancer. Cancers that may be treated include, for example without limitation, adult tumors/cancers and pediatric tumors/cancers. In some embodiments, the treatment can be in combination with one or more therapies against cancer selected from the group of antibodies therapy, chemotherapy, cytokines therapy, dendritic cell therapy, gene therapy, hormone therapy, laser light therapy and radiation therapy.

In some embodiments, treatment can be adminstered into subjects undergoing an immunosuppressive treatment. Indeed, embodiments of the invention rely on cells or a population of cells, which have been made resistant to at least one immunosuppressive agent due to the inactivation of a gene encoding a receptor for such immunosuppressive agent. In this aspect, the immunosuppressive treatment should help the selection and expansion of the T cells according to the invention within the subject. The administration of the cells or population of cells according to the invention may be carried out in any convenient manner, including by aerosol inhalation, injection, ingestion, transfusion, implantation or transplantation. The compositions described herein may be administered to a subject subcutaneously, intradermaliy, intratumorally, intranodally, intramedullary, intramuscularly, by intravenous or intralymphatic injection, or intraperitoneally. In some embodiments, the cell compositions of the invention are administered by intravenous injection.

In some embodiments, the administration of the cells or population of cells can comprise administration of, for example, about 10⁴ to about 10⁹ cells per kg body weight including all integer values of cell numbers within those ranges. In some embodiments the administration of the cells or population of cells can comprise administration of about 10⁵ to 10⁶ cells per kg body weight including all integer values of cell numbers within those ranges. The cells or population of cells can be administered in one or more doses. In some embodiments, said effective amount of cells can be administered as a single dose. In some embodiments, said effective amount of cells can be administered as more than one dose over a period time. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administered will be dependent upon the age, health and weight of the recipient, kind of concurrent treatment, if any, frequency of treatment and the nature of the effect desired. In some embodiments, an effective amount of cells or composition comprising those cells are administered parenterally. In some embodiments, administration can be an intravenous administration. In some embodiments, administration can be directly done by injection within a tumor.

In some embodiments of the invention, cells are administered to a subject in conjunction with (e.g., before, simultaneously or following) any number of relevant treatment modalities, including but not limited to treatment with agents such as monoclonal antibody therapy, CCR2 antagonist (e.g., INC-8761), antiviral therapy, cidofovir and interleukin-2, Cytarabine (also known as ARA-C) or nataliziimab treatment for MS patients or efaliztimab treatment for psoriasis patients or other treatments for PML patients. In some embodiments, target-specific CAR-T cells described herein with enhanced signaling, such as DLL3 CAR T cells described herein are administered to a subject in conjunction with one or more of the following: an anti-PD-1 antibody (e.g., nivolumab, pembrolizumab, or PF-06801591), an anti-PD-L1 antibody (e.g., avelumab, atezolizumab, or durvalumab), an anti-OX40 antibody (e.g., PF-04518600), an anti-4-1BB antibody (e.g., PF-05082566), an anti-MCSF antibody (e.g., PD-0360324), an anti-GITR antibody, and/or an anti-TIGIT antibody. In some embodiments, DLL3-specific CAR-T cells with enhanced signaling comprising the amino acid sequence shown in SEQ ID NOs: 43, 46 or 49, or other target-specific CAR-T cells, are administered to a subject in conjunction with anti-PD-L1 antibody avelumab. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and/or irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993). In further embodiments, the T cells of the invention may be used in combination with Receptor Tyrosine Kinase inhibitors such as Midostaurin and Sunitinib, mTOR inhibitors such as Rapamacyn and Everolimus, epigenetic modulators such as Vormostat, proteasome inhibitors such as Bortezomib, immunomodulatory agents such as lenalidomide, Hedgehog inhibitors such as Erismodegib and PF-04449913 or Isocitrate Dehydrogenase (IDH) inhibitors such as AG-120 and AG-221. In a further embodiment, the cell compositions of the invention are administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In some embodiments, the cell compositions of the invention are administered following B-cell ablative therapy such as agents that react with CD20, e.g., Rituxan. For example, In some embodiments, subjects may undergo standard treatment with high dose chemotherapy followed by peripheral blood stem cell transplantation. In certain embodiments, following the transplant, subjects receive an infusion of the expanded immune cells of the invention. In some embodiments, expanded cells are administered before or following surgery.

In some embodiments, provided are methods for depleting engineered immune cells of the invention as described herein from a subject adminstered with said cells. Depletion can be by inhibition or elimination.

In one aspect, a method for depleting engineered immune cells expressing a recombinant antigen receptor of the invention e.g. CAR of the invention comprising an epitope specific for a monoclonal antibody comprises contacting said engineered immune cell with a monoclonal antibody specific for the epitope.

In some embodiments, a method for depleting from a subject administered with engineered immune cells of the invention comprising an epitope specific for a monoclonal antibody comprises administering to the subject a monoclonal antibody specific for the epitope. In these embodiments, administration of the monoclonal antibody specific for the epitope present in the extracellular domain of the recombinant antigen receptor e.g. CAR to the subject eliminates or inhibits the activity of engineered recombinant antigen receptor-expressing e.g. CAR-expressing immune cells from the subject. In one aspect, depletion of engineered immune cells allows for recovery of an endogenous population of cells that express the target antigen of the recombinant antigen receptor of the invention.

In one aspect, the invention relates to a method for promoting recovery of endogenous target antigen-expressing cells in a subject administered with engineered immune cells expressing at cell surface a recombinant antigen receptor e.g. CAR of the invention comprising an epitope specific for a monoclonal antibody, the method comprising administering a monoclonal antibody specific for the epitope to the subject. In one aspect, the term “recovery” refers to increasing the number of endogenous target antigen-expressing cells. The number of endogenous target antigen-expressing cells may increase due to increase in proliferation of endogenous target-expressing cells and/or due to reduction in elimination of such endogenous cells by the engineered immune cells. In some embodiments, administration of the monoclonal antibody to the subject depletes the engineered immune cells and increases the number of endogenous target antigen-expressing cells in the subject. In one embodiment, administration of the monoclonal antibody to the subject increases the number of endogenous target antigen-expressing cells by at least about 10%, 15%, 20%, 25%, 30%, 35%, 40%, 45%, 50%, 55%, 60%, 65%, 70%, 75%, 80%, 85%, 90%, or 95%, compared to the number of endogenous target antigen-expressing cells prior to administration of the monoclonal antibody.

In one aspect, provided is a method for treating a target antigen-mediated condition in a subject, the method comprising: (a) administering to the subject engineered immune cells expressing at cell surface recombinant antigen receptors e.g. CARs of the invention comprising one or more epitopes specific for one or more monoclonal antibodies; and (b) subsequently depleting the engineered immune cells from the subject by administering one or more monoclonal antibodies specific for the epitope to the subject.

In some embodiments, the mAbs used in the method for depleting CAR-expressing engineered immune cells are selected from alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10, ustekinumab, and combinations thereof.

In some embodiments, the step of administering a monoclonal antibody to the subject comprises infusing the subject with the monoclonal antibody. In some embodiments, the amount of epitope-specific mAb administered to the subject is sufficient to eliminate at least 20%, 30%, 40%, 50%, 60%, 70%, 80% or 90% of the engineered immune cell in the subject.

In some embodiments, the step of administering a monoclonal antibody to the subject comprises infusing the subject with 375 mg/m² of rituximab, once or several times weekly.

In some embodiments, when immune cells expressing a CAR comprising an mAb-specific epitope (CAR-expressing immune cells) are depleted in a CDC assay using epitope-specific mAb, the amount of viable engineered immune cells decreases, e.g. by at least 10%, 20%, 30%, 40%, 50%, 60%, 70%, 80% or 90%.

In some embodiments, a cytotoxic drug is coupled to the epitope-specific mAbs which are used to deplete the engineered immune cells. By combining targeting capabilities of monoclonal antibodies with the cell-killing ability of cytotoxic drugs, antibody-drug conjugate (ADC) allows a sensitive discrimination between healthy and diseased tissue when compared to the use of the drug alone. Market approvals were received for several ADCs; the technology for making them—particularly on linkers—is abundantly presented in the following prior art (Payne, G. (2003) Cancer Cell 3:207-212; Trail et al (2003) Cancer Immunol. Immunother. 52:328-337; Syrigos and Epenetos (1999) Anticancer Research 19:605-614; Niculescu-Duvaz and Springer (1997) Adv. Drug Del. Rev. 26:151-172; U.S. Pat. No. 4,975,278).

In some embodiments, the epitope-specific mAb to be infused is conjugated beforehand with a molecule able to promote complement dependent cytotoxicity (CDC). Therefore, the complement system helps or complements the ability of antibodies to clear pathogens from the organism. When stimulated by one of several, is triggered an activation cascade as a massive amplification of the response and activation of the cell-killing membrane attack complex. Different molecules may be used to conjugate the mAb, such as glycans (Courtois, A, Gac-Breton, S., Berthou, C, Guezennec, J., Bordron, A. and Boisset, C. (2012), Complement dependent cytotoxicity activity of therapeutic antibody fragments is acquired by immunogenic glycan coupling, Electronic Journal of Biotechnology ISSN: 0717-3458; http://www.ejbiotechnology.info DOI: 10.2225/vol15-issue5).

### Kits

The invention also provides kits for use in the instant methods. Kits of the invention include one or more containers comprising a polynucleotide encoding a recombinant antigen receptor e.g. CAR of the present invention or an engineered immune cell comprising a polynucleotide encoding the recombinant antigen receptor e.g. CAR of the invention, and instructions for use in accordance with any of the methods of the invention described herein. Generally, these instructions comprise a description of administration of the engineered immune cell for the herein-described therapeutic treatments.

The instructions relating to the use of the engineered immune cells or antibodies as described herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a recombinant antigen receptor e.g. CAR of the present invention. The container may further comprise a second pharmaceutically active agent.

Kits may optionally provide additional components such as buffers and interpretive information. Normally, the kit comprises a container and a label or package insert(s) on or associated with the container.

The following examples are offered for illustrative purposes only, and are not intended to limit the scope of the invention in any way. Indeed, various modifications of the invention in addition to those shown and described herein will become apparent to those skilled in the art from the foregoing description and fall within the scope of the appended claims.

### EXAMPLES

### Example 1: Altering the Quality and Quantity of CD3ζ ITAMs as a Strategy to Improve CAR Signaling

A recombinant antigen receptor e.g. a CAR comprising a wildtype CD3ζ ITAM-containing domain has only 3 ITAMs, all of which derive from CD3ζ, whereas the naturally occurring TCR/CD3 holocomplex has 10 ITAMs comprising contributions from all four CD3 chains (delta, epsilon, gamma, and zeta). Reducing or increasing the number or diversity of ITAMs modulates both TCR and CAR function, underscoring the significance of these parameters. See, e.g., Bettini, M. L. et al. Cutting Edge: CD3 ITAM Diversity Is Required for Optimal TCR Signaling and Thymocyte Development. J. Immunol. 199, 1555-1560 (2017); Feucht, J. et al. Calibration of CAR activation potential directs alternative T cellfates and therapeutic potency. Nature Medicine 25, 82-88 (2019); Majzner, R. G. et al. Low CD19 Antigen Density Diminishes Efficacy of CD19 CAR T Cells and Can be Overcome By Rational Redesign of CAR Signaling Domains. Blood 132, 963 (2018). Thus, the quantity and quality of ITAMs and the complement of proximate signaling mediators differ between CARs and TCRs.

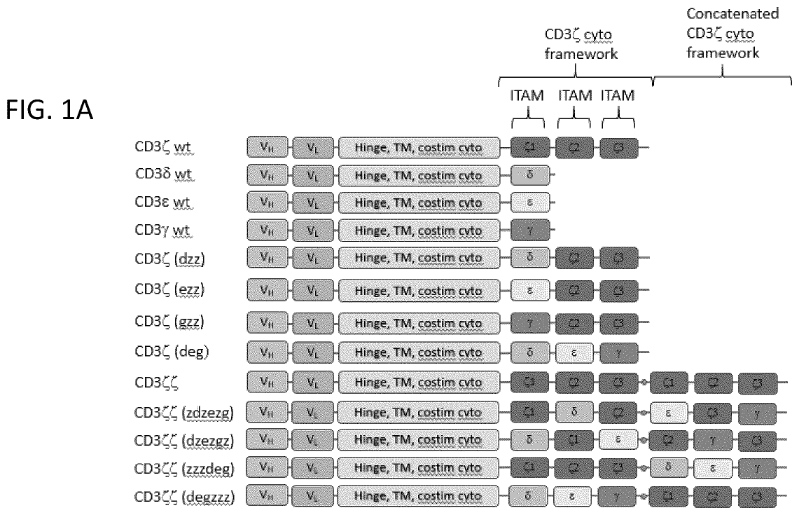

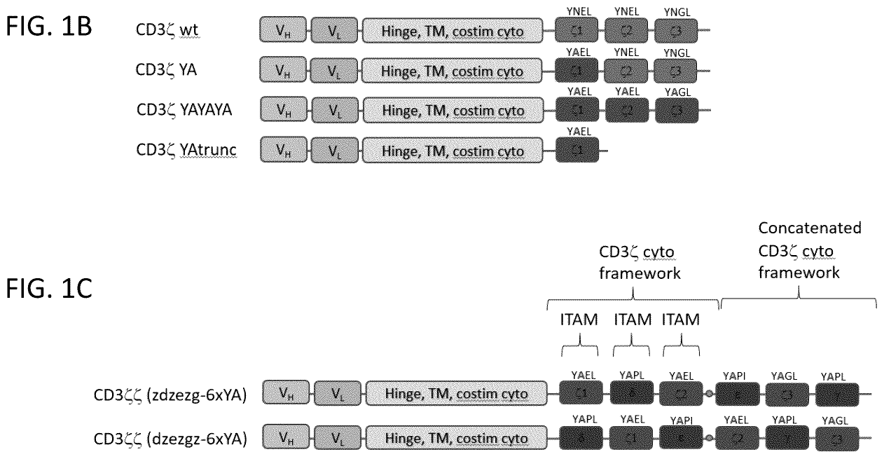

To test whether the sensitivity of CAR signaling can be improved by more closely mimicking TCR signaling, we altered the quality and quantity of CD3ζ ITAMs in a DLL3 CAR (10G1-K). In one set of constructs, the number of ITAMs affiliated with the CAR was decreased or increased (e.g. by truncating CAR after the first ITAM or by concatenating two copies of the CD3ζ cytoplasmic tail) (FIG. 1A). In addition, the character of the ITAMs in these constructs was varied by replacing them in the CD3ζ framework with ITAMs from other CD3 chains in various configurations (FIG. 1A). In a second set of constructs, the residue in the second position in the canonical YXX(L/I) ITAM motif was mutated to Ala, a mutation previously reported to reduce the degree of receptor clustering necessary to initiate signaling (FIG. 1B, Table 1). See, e.g., Sunder-Plassmann, R. et al. Functional analysis of immunoreceptor tyrosine-based activation motif (ITAM)-mediated signal transduction: The two YxxL segments within a single CD3ζ-TAM are functionally distinct. Eur. J. Immunol. 27, 2001-2009 (1997). We reasoned that this mutation may therefore facilitate signaling in response to low density tumor antigens. Constructs that combine the strategies described for FIGS. 1A & 1B were also created (FIG. 1C).

### Example 2: Overexpression of Downstream T Cell Signaling Mediators to Improve CAR Signaling

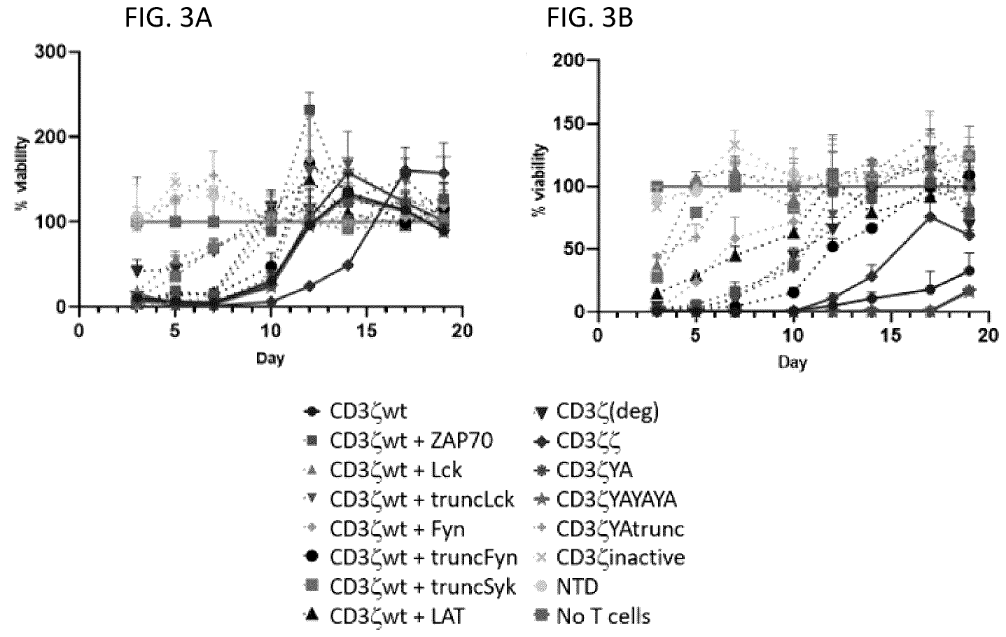

CARs employing the 4-1BB costimulatory domain inefficiently recruit downstream mediators of T cell signaling (e.g. ZAP70) (see Gudipati, V. et al. Inefficient CAR-proximal signaling blunts antigen sensitivity. Nat. Immunol. (2020). doi:10.1038/s41590-020-0719-0) and overexpression of these mediators (e.g. Lck) has been shown to boost CAR function (see Sun, C. et al. THEMIS-SHP1 Recruitment by 4-1BB Tunes LCK-Mediated Priming of Chimeric Antigen Receptor-Redirected T Cells. Cancer Cell 37, 216-225.e6 (2020)). We designed a series of constructs that employ an intervening 2A ribosomal skip sequence to couple CAR expression with overexpression of one of multiple downstream mediators of T cell signaling (see, e.g., Table 1, listing the following: SEQ ID NO: 1 (ZAP70), 2 (Lck), 4 (Fyn), 6 (Syk), 8 (LAT), and 9 (Unc119), SEQ ID NOs: 3 (Truncated Lck), 5 (Truncated Fyn), and 7 (Truncated Syk)). See FIGS. 3A-B.

### Example 3: Comparison of CAR Constructs with Different Quality and Quantity of ITAMs In Vitro

In this example, the constructs described in Example 1 were transduced in primary human T cells and tested for in vitro cytotoxic activity.

To make lentivirus expression constructs described in Example 1, HEK-293T cells were plated at 1.5 million cells per mL in 2 mL of DMEM (Gibco) supplemented with 10% FBS (Hyclone) per well of a 6-well plate on Day −1. On Day 0, the lentivirus was prepared by mixing together with lentiviral packaging vectors 1.5 ug psPAX2, 0.5 ug pMD2G, and 0.5 ug of the appropriate transfer CAR added to the DNA mix. The DLL3-specific CAR clone 10G1-K was used in this experiment. See WO2020/180591.

The mixture was incubated at room temperature for 20 minutes and the total volume of 500 uL was slowly added to the sides of the wells containing HEK-293T. Purified T cells were activated in X-Vivo-15 medium (Lonza) supplemented with 100 IU/mL human TL-2 (Miltenyi Biotec), 10% FBS (Hyclone), and human T TransAct (Miltenyi Biotec, Cat #130-111-160, 1:100 dilution). On Day 1, the media from each well of the 6-well plate was replaced with 2 mL per well of T cell transduction media, i.e., X-Vivo-15 supplemented with 10% FBS. On Day 2, T cells were resuspended at 0.4 million cells per mL in 1.5 mL of T cell transduction media per well of a Grex-24 plate (Wilson Wolf, cat #80192M). The lentiviral supernatants from HEK293T cells (about 1.5 ml) were harvested and passed through a 0.45 micron filter (EMD Millipore) to remove cell debris, and then added to the T cells along with 100 IU/mL human IL-2. On Day 5, 4.5 mL of T cell expansion media, i.e., X-Vivo-15 supplemented with 5% human AB serum (Gemini Bio) was added to each well of a Grex-24 plate. On Day 9 and Day 13, transduction efficiency was determined by detecting the percentage of T cells that express BFP and recognize recombinant DLL3 (Adipogen) using flow cytometry. Cells were expanded into larger flasks or G-Rex vessels (Wilson Wolf) as needed using T cell expansion media. On Day 14, DLL3 CAR-T cells were cryopreserved. Percentage of cells stained with recombinant DLL3 was normalized across clones right before cryopreservation.

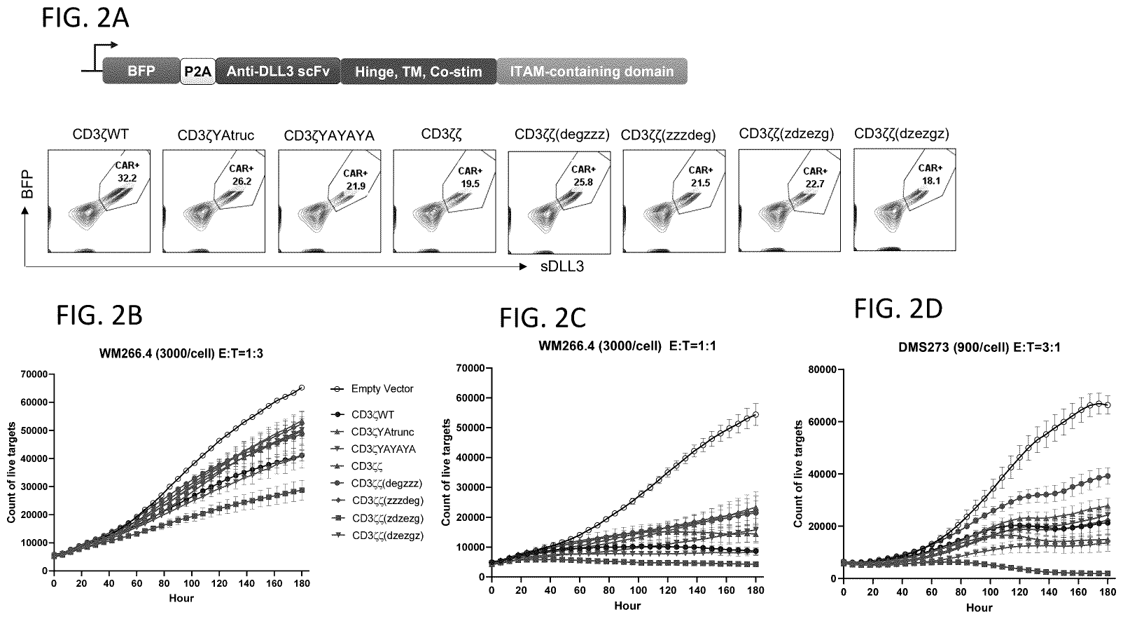

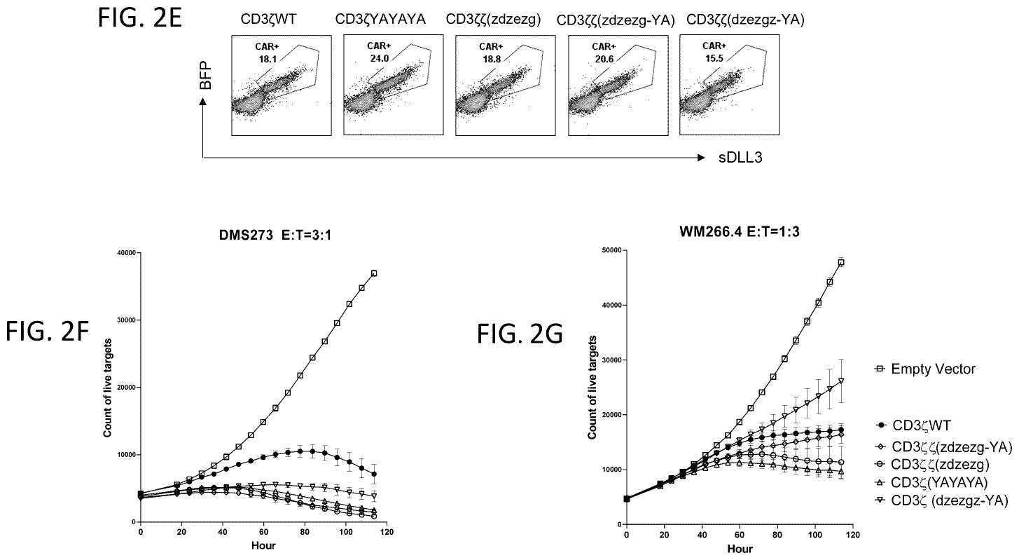

To determine the percentage of T cells that were successfully transduced with DLL3 CAR, T cells were first incubated with 1 ug/ml Flag tagged recombinant DLL3 (Adipogen) in PBS+1% BSA for 20 minutes at 4° C. Then cells were washed with PBS+1% BSA, stained with PE labelled anti-Flag antibodies (Biolegend, Cat #637310) and analyzed using flow cytometry. Examples of CAR T cells are shown in FIG. 2A-bottom panels. The results in FIG. 2A-bottom panels show that conventional (comprising wildtype CD3ζ ITAM-containing domain) or modified CARs (comprising a non-wildtype CD3ζ ITAM-containing domain) were expressed on the surface of primary T-cells. These results showed strong correlation between BFP expression and recombinant DLL3 staining, suggesting these constructs expressed properly and the proteins expressed had no major issues of folding or surface localization. The plots were gated on live CD3+ cells. The numbers on the plots are the percentage of cells that expressed each CAR construct. FIG. 2E shows the detection of conventional or modified CARs in a second human donor.

To compare the efficacy of conventional and modified CARs in a short-term kinetic killing assay, CAR T cells were incubated with DLL3 positive DMS273 (low antigen density) or WM266.4 (high antigen density) cells expressing nuclear GFP at indicated effector:target (E:T) ratio in T cell expansion media, i.e., X-Vivo-15 supplemented with 5% human AB serum (Gemini Bio). The tissue culture plates were placed in Incucyte and the number of GFP positive target cells was counted every 6 hours. FIGS. 2B-2D show in one human T cell donor, several constructs described in Example 1 improved CAR T cytotoxicity against both DMS273 and WM266.4 target cells (relative to CAR comprising a wildtype CD3ζ ITAM-containing domain). FIG. 2F-2G show that in CAR T cells derived from a second human donor, several constructs described in Example 1 also improved CAR T cell cytotoxicity (relative to CAR comprising a wildtype CD3ζ ITAM-containing domain).

To compare the efficacy of CARs described in Example 1 and Example 2 in a long-term cytotoxicity assay, CAR-T cells were exposed repeatedly to the target DLL3 every 2 to 3 days to promote CAR-T cells proliferation. On the first day of the assay, 5,000 firefly luciferase labelled WM266.4 or DMS273 cells were seeded in 96-well plates with black wall and flat clear bottom in 100 ul X-Vivo-15 medium with 5% of human serum. After target cells attached to the bottom of the plates, CAR T cells were thawed and added to plated target cells in X-VIVO medium with 5% of human serum. Every 2 to 3 days thereafter, 100 μl medium containing CAR T cells were transferred to freshly plated target cells and percentage lysis of previously plated target cells were determined using one-glo assay system (Promega). Each condition was assayed in 3 replicates. Average percentage of lysis and standard deviation were plotted in FIGS. 3A-3B. The experimental data of serial killing assay shows that after repeated exposure of CAR T cells to DLL3 positive target cells, some of the constructs performed better than the conventional CD3ζWT construct.

### Example 4: Insertion of Lck Recruitment Motifs (LRM) as a Strategy to Enable Recruitment of Lck to CAR Synapses in a More TCR-Like Manner

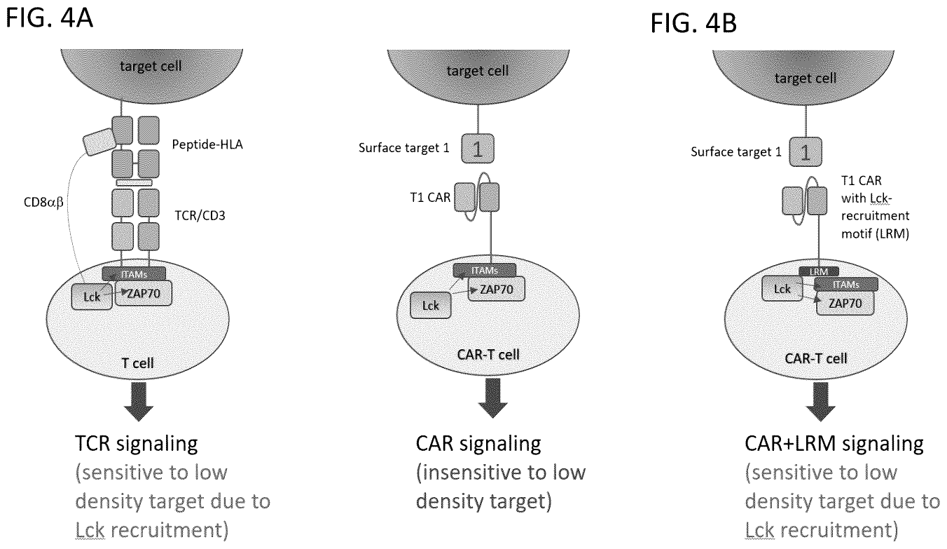

Synapse formation for CARs does not require co-receptor (CD8 or CD4) involvement. As a result, the co-receptor-associated Lck kinase that is central to TCR signaling is coordinated with target recognition for the TCR synapse but is not for the CAR synapse (FIG. 4A). See Davenport, A. J. et al. Chimeric antigen receptor T cells form nonclassical and potent immune synapses driving rapid cytotoxicity. Proc. Natl. Acad. Sci. U.S.A 115, E2068-E2076 (2018).

To facilitate Lck-mediated signaling events in CAR T cells, constructs were designed to deliver a CAR with a cytoplasmic domain modified to include a co-receptor-derived sequence motif that recruits T cell signaling mediators (FIG. 4B). In this example, a Lck recruitment motif (LRM) (derived, for example, from CD8, CD4, or CD28) was inserted between cytoplasmic domains and the intracellular domain of the CAR, between the co-stimulatory domain and the ITAM containing domain, or at the C-terminus of the CAR cytoplasmic tail, enabling directed recruitment of Lck to the CAR receptor (FIG. 5A). We hypothesized that the constructs will promote more efficient phosphorylation of the CAR ITAMs and ZAP70, and thus more efficient signaling of the CAR upon antigen binding and synapse formation. Exemplary LRM amino acid sequences of SEQ ID NOs: 55 (CD8LRM-1), 56 (2×CD8LRM-1), 57 (CD8LRM-2), 58 (CD28LRM), 59 (CD28LRMY3), 64 (CD4LRM)) are shown in Table 1.

### Example 5: Comparison of CAR Constructs with LRM in an In Vitro Cytotoxicity Assay

In this example, the constructs described in Example 4 were transduced in primary human T cells tested for in vitro cytotoxic activity. Lentivirus encoding constructs described in Example 4 and primary T cells transduced with these lentiviruses were produced using methods described in Example 3.

The results in FIG. 5B show that conventional CAR or CARs with an LRM insertion were expressed on the surface of primary T-cells. These T cells showed strong correlation between BFP expression and recombinant DLL3 staining, suggesting these constructs expressed properly and the proteins expressed had no major issues of folding or surface localization. The plots were gated on live CD3+ cells. The numbers on the plots are the percentage of cells expressing each CAR construct.

To compare the efficacy of conventional and modified CARs in a short-term kinetic killing assay, CAR T cells were incubated with DLL3 positive DMS273 (low antigen density) or WM266.4 (high antigen density) cells expressing nuclear GFP at indicated effector:target (E:T) ratio in T cell expansion media, i.e., X-Vivo-15 supplemented with 5% human AB serum (Gemini Bio). The tissue culture plates were placed in Incucyte and the number of GFP positive target cells was counted every 6 hours. FIG. 5C shows constructs with an LRM inserted between transmembrane domain and 4-1BB cytoplasmic domain did not perform as well as the conventional CAR construct. On the other hand, FIGS. 5D-5E show that several CAR constructs with an LRM inserted at the C-terminus of the CAR intracellular domain demonstrated comparable or better cytotoxicity against both DMS273 and WM266.4 target cells. The DLL3-specific CAR clone 10G1-K was used in this experiment.

### Example 6: LckCARs as a Strategy to Boost Recruitment of Lck to the CAR Synapse and to Enable Combinatorial CAR Targeting

A major obstacle to applying CAR T therapies to solid tumors has been the lack of suitable targets. Ideally, solid tumor targets can be identified that are expressed at high and relatively uniform levels across tumor cells but that are not expressed or expressed only at low levels on healthy tissues (as CD19 is in non-solid tumors). If solid tumor antigens cannot be identified with this auspicious confluence of characteristics, CAR design should accommodate the shortcomings of the less than ideal solid tumor target antigens. For instance, CAR-mediated targeting of solid tumor antigens that are also expressed in normal tissues may require combinatorial targeting (e.g. via logic gates) to de-risk these liabilities.

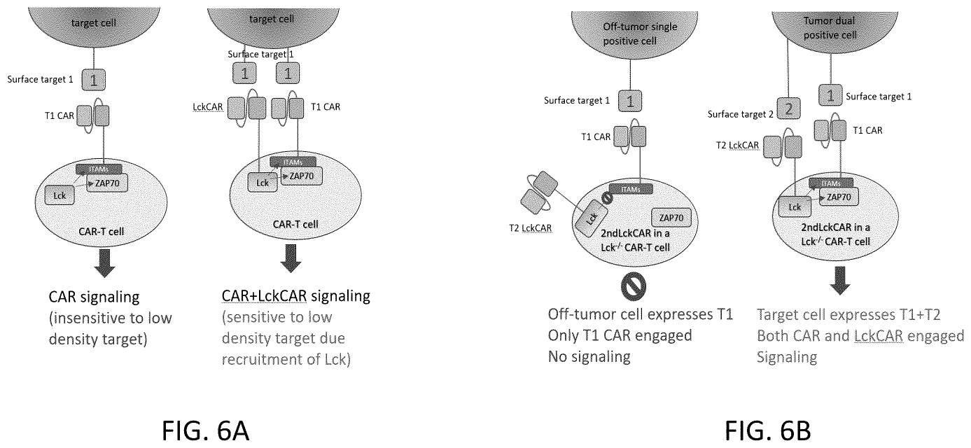

To facilitate combinatorial targeting, a non-standard “CAR” or a second recombinant antigen receptor was designed, in which the intracellular domain of the non-standard CAR comprises, instead of domains that provide signal 1 and/or signal 2, a downstream mediator of T cell signaling or a functional variant thereof. As an example, such a non-standard “LckCAR” is shown in FIG. 6A. The LckCAR fuses an antigen recognition domain via the CD8 (or alternative) transmembrane domain to a protein comprising a Lck-recruitment motif (e.g. CD8 cytoplasmic tail) or directly to the Lck protein itself. Targeting of this LckCAR to the same target as a target-specific CAR may increase Lck recruitment to the synapse, juxtaposing Lck with ITAM substrates and thereby augmenting signaling efficiency (FIG. 6A). Targeting of the LckCAR to a second tumor-associated target antigen distinct from that recognized by the co-expressed CAR may enable AND-gated combinatorial targeting of solid tumor targets, thereby improving safety (FIG. 6B). There are many potential variations on this theme employing fusions of an extracellular antigen binding domain to different downstream T cell signaling mediators, such as ZAP70 and LAT.

### Example 7: Comparison of CAR Constructs with Different ITAMs or LRM in the Context of DLL3 CAR 4H8-R2S

In this example, the constructs described in FIG. 1 were evaluated in the context of DLL3 CAR clone 4H8-R2S (see WO2020/180591) to demonstrate that the designs are broadly applicable.

To make lentivirus encoding constructs described in Example 1, HEK-293T cells were plated at 1.5 million cells per mL in 2 mL of DMEM (Gibco) supplemented with 10% FBS (Hyclone) per well of a 6-well plate on Day −1. On Day 0, the lentivirus was prepared by mixing together with lentiviral packaging vectors 1.5 ug psPAX2, 0.5 ug pMD2G, and 0.5 ug of the appropriate transfer CAR added to the DNA mix. The mixture was incubated at room temperature for 20 minutes and the total volume of 500 uL was slowly added to the sides of the wells containing HEK-293T. Purified T cells were activated in X-Vivo-15 medium (Lonza) supplemented with 100 IU/mL human IL-2 (Miltenyi Biotec), 10% FBS (Hyclone), and human T TransAct (Miltenyi Biotec, Cat #130-111-160, 1:100 dilution). On Day 1, the media from each well of the 6-well plate was replaced with 2 mL per well of T cell transduction media, i.e., X-Vivo-15 supplemented with 10% FBS. On Day 2, T cells were resuspended at 0.4 million cells per mL in 1.5 mL of T cell transduction media per well of a Grex-24 plate (Wilson Wolf, cat #80192M). The lentiviral supernatants from HEK293T cells (about 1.5 ml) were harvested and passed through a 0.45 micron filter (EMD Millipore) to remove cell debris, and then added to the T cells along with 100 IU/mL human IL-2. On Day 5, 4.5 mL of T cell expansion media, i.e., X-Vivo-15 supplemented with 5% human AB serum (Gemini Bio) was added to each well of a Grex-24 plate. On Day 9 and Day 13, transduction efficiency was determined by detecting the percentage of T cells that recognize recombinant Flag-DLL3 (Adipogen) using flow cytometry. Cells were expanded into larger flasks or G-Rex vessels (Wilson Wolf) as needed using T cell expansion media. On Day 14 or Day 16, DLL3 CAR-T cells were cryopreserved. Percentage of cells stained with recombinant DLL3 was normalized across clones right before cryopreservation.

To determine the percentage of T cells that were successfully transduced with the DLL3 CAR, T cells were first incubated with 1 ug/ml Flag tagged recombinant DLL3 (Adipogen) in PBS+1% BSA for 20 minutes at 4° C. The cells were then washed with PBS+1% BSA, stained with PE labelled anti-Flag antibodies (Biolegend, Cat #637310) and analyzed using flow cytometry.

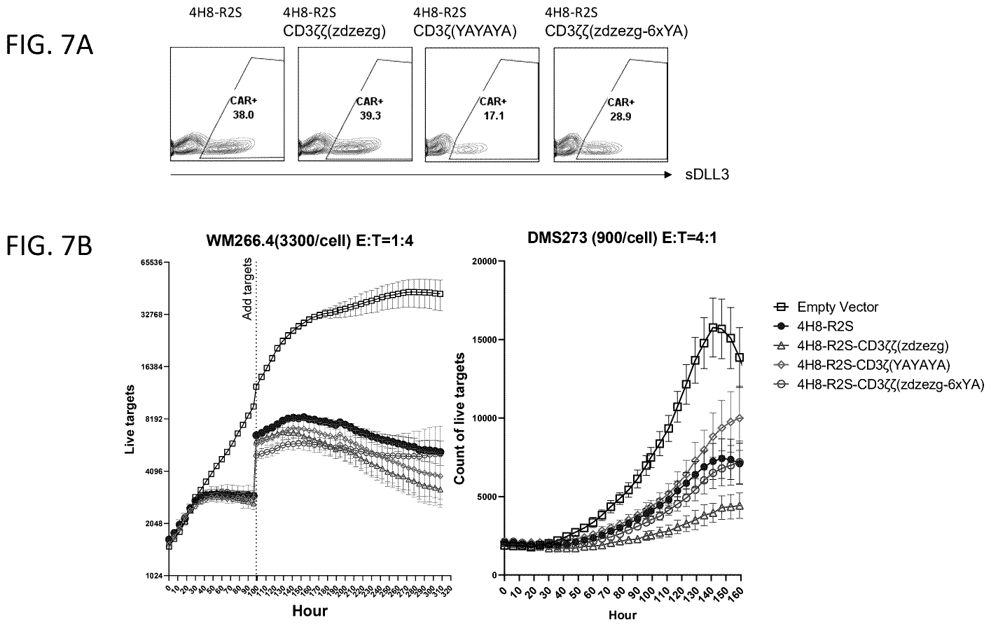

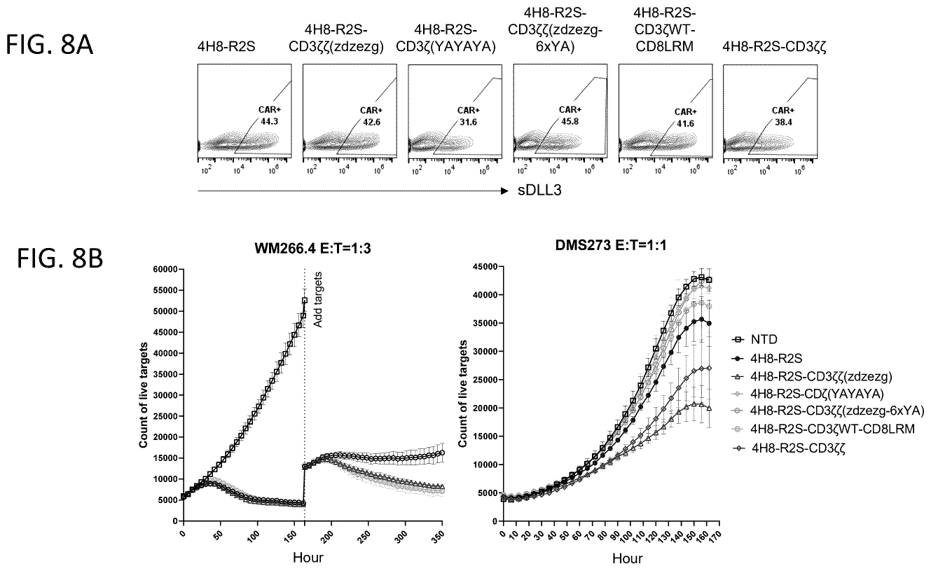

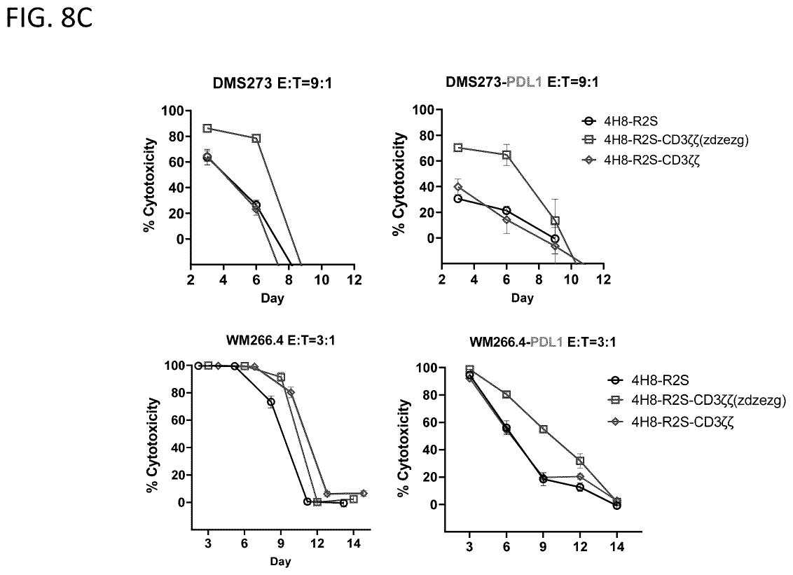

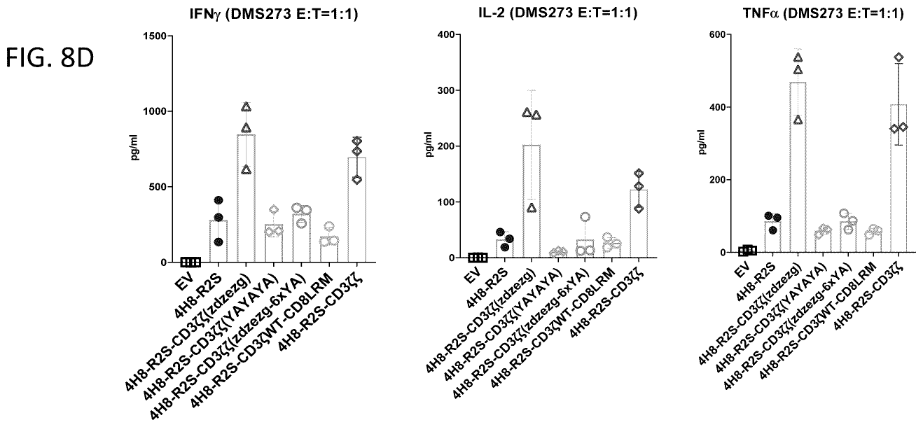

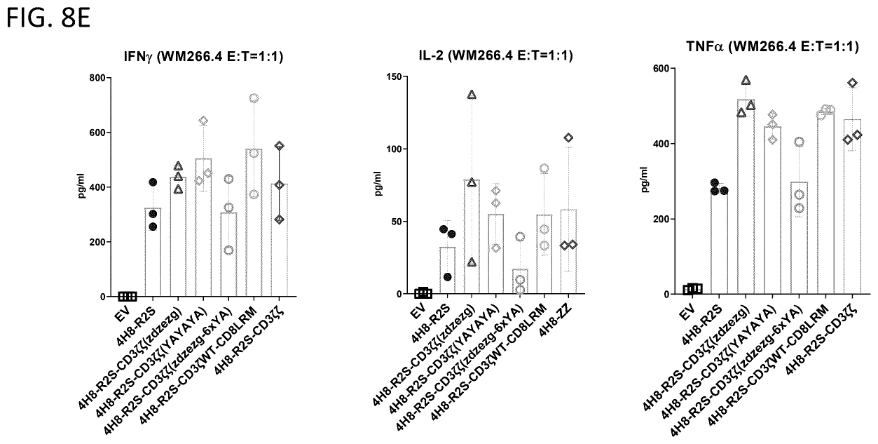

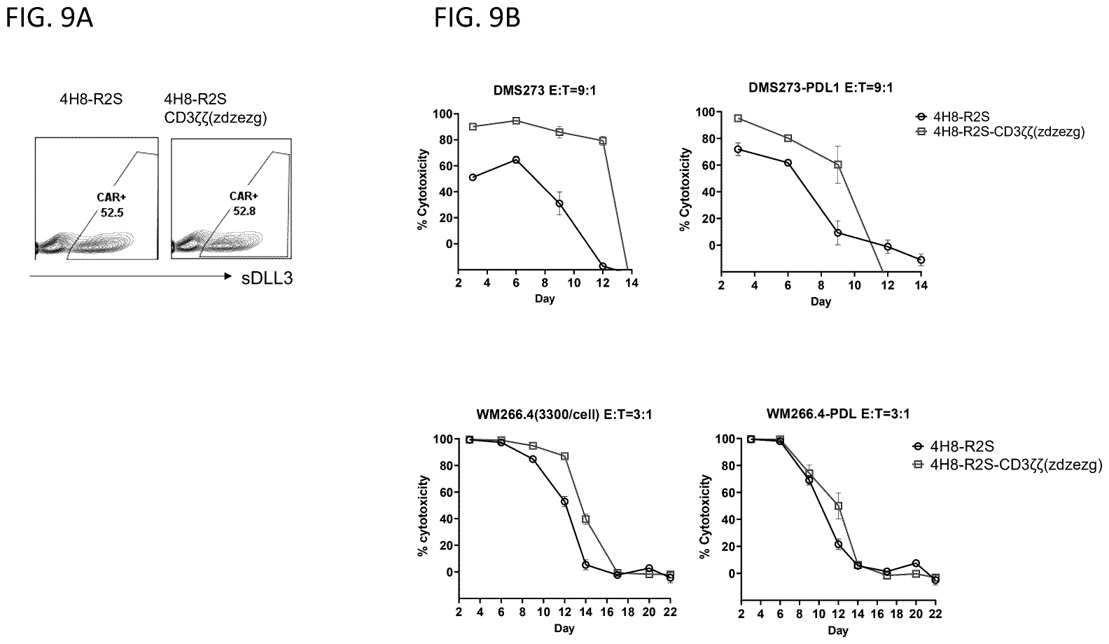

FIGS. 7A-B, FIGS. 8A-E and FIGS. 9A-B show series of results of CAR T cells produced from three different human donors, respectively. FIGS. 7A, 8A and 9A show flow cytometry data exhibiting expression of various DLL3 CAR 4H8-R2S constructs on the surface of CAR T cells generated from three separate human donors. The plots are gated on live CD3+ cells. The numbers indicate the percentage of cells expressing each CAR construct. Most of the modified CAR T cells have similar percentage of CAR T cells compared to control CAR T cells. The activity data were normalized against transduction efficiency.

To compare the efficacy of various DLL3 CAR 4H8-R2S in a short-term kinetic killing assay, CAR T cells were incubated with DLL3 positive DMS273 cells (low antigen density cell line) or WM266.4 cells (high antigen density cell line) expressing nuclear GFP at indicated effector:target (E:T) ratio in RPMI (Gibco) supplemented with 10% FBS (Hyclone). The tissue culture plates were placed in Incucyte and the number of GFP positive target cells was counted every 6 hours. FIG. 7B shows CD3ζζ(zdzezg) construct in the context of DLL3 CAR 4H8-R2S performed better than the DLL3 CAR 4H8-R2S CD3ζWT construct, while CARs with the CD3ζ(YAYAYA) and CD3ζζ(zdzezg-6×YA) modifications were comparable to CD3ζWT. FIG. 8B shows results of cytotoxicity assay where CAR T were prepared using primary T cells from a separate human donor. The results show that CD3ζζ(zdzezg) and CD3ζζ constructs were superior to CD3ζWT in cytotoxic activity.

To compare the efficacy of CARs in a long-term cytotoxicity assay, CAR-T cells were exposed repeatedly to their targets every 2 to 3 days causing the CAR-T cells to undergo proliferation and in certain cases, differentiation and, in certain cases, exhaustion. PD-L1/PD-1 pathway has been shown to downregulate TCR and CAR signaling. To test if modified CAR constructs can resist the inhibition from PD-L1/PD-1 axis, 5,000 firefly luciferase labelled parental or PD-L1 overexpressed WM266.4 and DMS273 cells were seeded in 96-well plates with black wall and flat clear bottom in 50 ul RPMI (Gibco) supplemented with 10% FBS (Hyclone). After target cells attached to the bottom of the plates, CAR T cells were thawed and added to plated target cells. Every 2 to 3 days thereafter, 100 μl medium containing CAR T cells were transferred to freshly plated target cells and percentage lysis of previously plated target cells were determined using one-glo assay system (Promega). Each condition was assayed in 5 replicates. Average percentage of lysis and standard deviation were plotted. FIG. 8C and FIG. 9B show data of CAR T cells from two separate human donors. The results show that after repeated exposure of CAR T cells to DLL3 positive target cells, CD3ζζ(zdzezg) construct performed better than the CD3ζWT construct in the context of DLL3 CAR 4H8-R2S, against both parental DLL3-expressing target cells or target cells further overexpressing PD-L1. The CD3ζζ construct was superior to the CD3ζWT construct in some settings and comparable to CD3ζWT in other settings.

To measure cytokines secreted from DLL3 CAR-T cells, CAR-T cells were incubated with WM266.4 and DMS273 at effector:target (E:T) ratio of 1:1 in RPMI (Gibco) supplemented 10% FBS (Hyclone). 24 hours later, tissue culture supernatant was collected and the levels of 3 cytokines [interferon gamma (IFN-7), tumor necrosis factor alpha (TNF-α), and IL-2] in the supernatants were measured using human proinflammatory tissue culture 9-plex assay (MSD) following manufacturer's protocol.

FIG. 8D shows CD3ζζ(zdzezg) and CD3ζζ CAR T cells secreted higher levels of IFN-γ, TNF-α and IL-2 than CD3ζWT CAR T cells when co-cultured with DMS273 (low DLL3 density) cell line, n=3.

FIG. 8E shows CD3ζζ(zdzezg), CD3ζζ, CD3ζ(YAYAYA), and CD3ζWT-CD8LRM secret comparable or higher levels of cytokines than CD3ζWT CAR T cells when co-cultured with WM266.4 (high DLL3 density) cell line, n=3.

### Example 8: Comparison of CAR Constructs with Different Quality and Quantity of ITAMs in the Context of DLL3 CAR 2G1-RSR

In this example, the constructs described in FIG. 1 were evaluated in the context of DLL3 CAR clone 2G1-RSR (see WO2020/180591) to demonstrate the designs are broadly applicable.

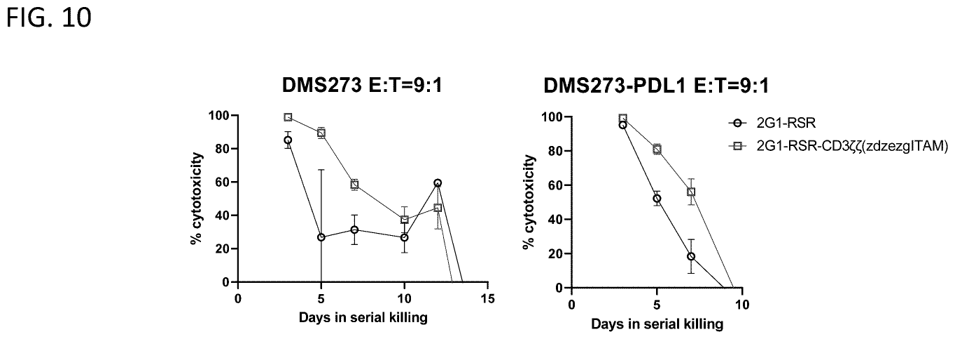

2G1-RSR DLL3 CAR T cells were produced as described and detected in Example 7. To compare the efficacy of CARs in a long-term cytotoxicity assay, CAR-T cells were exposed repeatedly to their targets every 2 to 3 days causing the CAR-T cells to undergo proliferation and in certain cases, differentiation and exhaustion. On the first day of the assay, 5,000 firefly luciferase labelled parental DMS273 or DMS273 overexpressing PD-L1 were seeded in 96-well plates with black wall and flat clear bottom in 50 ul RPMI (Gibco) supplemented with 10% FBS (Hyclone). After target cells attached to the bottom of the plates, CAR T cells were thawed and added to plated target cells. Every 2 to 3 days thereafter, 100 μl medium containing CAR T cells were transferred to freshly plated target cells and percentage lysis of previously plated target cells were determined using one-glo assay system (Promega). Each condition was assayed in 3 replicates. FIG. 10 shows data of long-term cytotoxicity assay, the CD3ζζ(zdzezg) construct performed better than the CD3ζWT construct in the context of DLL3 CAR 2G1-RSR on both parental and PD-L1-overexpressing DMS273 cells.

### REFERENCES

Bettini, M. L. et al. Cutting Edge: CD3 ITAM Diversity Is Required for Optimal TCR Signaling and Thymocyte Development. J. Immunol. 199, 1555-1560 (2017).
Feucht, J. et al. Calibration of CAR activation potential directs alternative T cell fates and therapeutic potency. Nature Medicine 25, 82-88 (2019).
Majzner, R. G. et al. Low CD19 Antigen Density Diminishes Efficacy of CD19 CAR T Cells and Can be Overcome By Rational Redesign of CAR Signaling Domains. Blood 132, 963 (2018).
Sunder-Plassmann, R., Lialios, P., Madsen, M., Koyasu, S. & Reinherz, E. L. Functional analysis of immunoreceptor tyrosine-based activation motif (ITAM)-mediated signal transduction: The two YxxL segments within a single CD3ζ-ITAM are functionally distinct. Eur. J. Immunol. 27, 2001-2009 (1997).
Gudipati, V. et al. Inefficient CAR-proximal signaling blunts antigen sensitivity. Nat. Immunol. (2020). doi:10.1038/s41590-020-0719-0
Sun, C. et al. THEMIS-SHP1 Recruitment by 4-1BB Tunes LCK-Mediated Priming of Chimeric Antigen Receptor-Redirected T Cells. Cancer Cell 37, 216-225.e6 (2020).
Davenport, A. J. et al. Chimeric antigen receptor T cells form nonclassical and potent immune synapses driving rapid cytotoxicity. Proc. Natl. Acad. Sci. U.S.A 115, E2068-E2076 (2018).

1-40. (canceled)

41. A method of treating a disease or condition comprising administering to a patient in need thereof an effective amount of an engineered immune cell comprising a recombinant antigen receptor comprising an extracellular antigen binding domain, a transmembrane domain, and an intracellular domain that comprises a co-stimulatory domain and an ITAM-containing domain, wherein the ITAM-containing domain comprises from N-terminus to C-terminus (a) CD3z1 ITAM, CD3d ITAM, CD3z2 ITAM, CD3e ITAM, CD3z3 ITAM, CD3g ITAM, (b) CD3z1 (YAEL (SEQ ID NO: 152)) ITAM, CD3z2 (YAEL (SEQ ID NO: 152)) ITAM, CD3z3 (YAGL (SEQ ID NO: 153)) ITAM, or (c) CD3z1 (YAEL (SEQ ID NO: 152)) ITAM, CD3d (YAPL (SEQ ID NO: 154)) ITAM, CD3z2 (YAEL (SEQ ID NO: 152)) ITAM, CD3e (YAPI (SEQ ID NO: 155)) ITAM, CD3z3 (YAGL (SEQ ID NO: 153)) ITAM, CD3g (YAPL (SEQ ID NO: 154)) ITAM.

42. The method of claim 41, wherein the recombinant antigen receptor is a chimeric antigen receptor (CAR).

43. The method of claim 41, wherein the antigen binding domain comprises a heavy chain variable domain (VH) and a light chain variable domain (VL).

44. The method of claim 41, wherein the co-stimulatory domain comprises 4-1BB co-stimulatory domain.

45. The recombinant antigen receptor of claim 41, wherein the intracellular domain comprises an amino acid sequence selected from the group consisting of SEQ ID NOs:
30, 35 and 37.

46. The recombinant antigen receptor of claim 45, wherein the intracellular domain comprises an amino acid sequence selected from the group consisting of SEQ ID NOs: 30 and 37.

47. The recombinant antigen receptor of claim 45, wherein the intracellular domain comprises an amino acid sequence of SEQ ID NO:35.

48. A method of treating a disease or condition comprising administering to a patient in need thereof an effective amount of an engineered immune cell comprising a recombinant antigen receptor comprising an extracellular antigen binding domain, a transmembrane domain, and an intracellular domain that comprises a 4-1BB co-stimulatory domain, a CD3z ITAM-containing domain, and further comprising an additional Lck recruiting motif (LRM), wherein the additional LRM is a CD8 LRM comprising, consisting of or consisting essentially of the amino acid sequence of SEQ ID NO: 56.

49. A method of treating a disease or condition comprising administering to a patient in need thereof an effective amount of an engineered immune cell comprising a first recombinant antigen receptor and a second recombinant antigen receptor, wherein the first recombinant antigen receptor comprises a CAR and the second recombinant receptor comprises an extracellular antigen binding domain and an intracellular domain that comprises a downstream mediator of T cell signaling, or a functional variant thereof.

50. The method of claim 49, wherein the antigen binding domain of the second recombinant antigen receptor binds to the same or different antigen as the first recombinant antigen receptor.

51. The method of claim 49, wherein the downstream mediator of T cell signaling is ZAP70, Lck, Fyn, Syk, LAT, or UNC119, or a functional variant thereof.

52. The method of claim 51, wherein the downstream mediator of T cell signaling comprises an amino acid sequence selected from the group consisting of SEQ ID NOs: 1-9.

53. The method of claim 49, wherein the CAR comprises an intracellular domain that comprises an amino acid sequence selected from SEQ ID NOs: 26-38, 55-59, and 64-70.

54. The method of claim 49, wherein the CAR comprises an amino acid sequence selected from SEQ ID NOs: 91-145, with or without a signal peptide.

55. The method of claim 49, wherein the immune cell is a T cell.

56. The method of claim 55, wherein the immune cell comprises one or more genomic modifications to the TCRa gene.

57. The method of claim 49, wherein the antigen binding domain of the recombinant antigen receptor binds to DLL3.
